SEQUENCE LISTING (23 sequences)

SEQ ID NO: 1 - DNA, 20 bp, Homo sapiens
accctcaagttttgcccttt

SEQ ID NO: 2 - DNA, 20 bp, Homo sapiens
gatcctccagcagcaacttc

SEQ ID NO: 3 - DNA, 20 bp, Homo sapiens
gggcctcaaaggaaagaatc

SEQ ID NO: 4 - DNA, 20 bp, Homo sapiens
taccagttggggaactctgc

SEQ ID NO: 5 - DNA, 20 bp, Homo sapiens
gacagcctgtgttcgaggat

SEQ ID NO: 6 - DNA, 20 bp, Homo sapiens
tggatccatttcctcaaagg

SEQ ID NO: 7 - DNA, 20 bp, Homo sapiens
atgctgcttcgacatctcct

SEQ ID NO: 8 - DNA, 20 bp, Homo sapiens
aaccaatgcgagatcctgac

SEQ ID NO: 9 - DNA, 19 bp, Homo sapiens
ttcgtattgcgccgctaga

SEQ ID NO: 10 - DNA, 20 bp, Homo sapiens
ctttcgctctggtccgtctt

SEQ ID NO: 11 - RNA, 35 nt, Homo sapiens
ugagguaguagguuguauaguuuuagggucacacc

SEQ ID NO: 12 - RNA, 37 nt, Homo sapiens
caccacugggagauaacuauacaaucuacugucuucu

SEQ ID NO: 13 - DNA, 17 bp, Homo sapiens
ttcttatcggtgtgcct

SEQ ID NO: 14 - DNA, 9851 bp, Homo sapiens
  CDS (280)..(6000)
ggtcggggcgcggcgcggtgcggcgcgggcggctccgggcccaggtgaatggagtaacctgacagcgggaacgagacgacggcgagcggcaggaaatggcggcggcggcggcggcgccgggcggcaccgggaggcctgggctgtgacgcgcgcgccggagcggggtccgatggttctggaaggcccgcggcgcccagtgctgcagtaagctgtgctagaacaaaatgcaatgaaagaaacactggatgattgaaaagccctgctttgcagcccctcagcatggcaggcctgcagctcatgacccctgcttcctcaccaatgggtcctttctttggactgccatggcaacaagaagcaattcatgataacatttatacgccaagaaaataccaggttgagctgctggaagcagctctggaccataacacaattgtgtgcttgaacactggctcagggaagacgttcatcgcggtcctgctcaccaaagagctggcccatcagatcaggggcgacctcaacccgcatgcaaaaaggaccgtgttcctcgtcaactctgcaaaccaggttgctcaacaagtgtcagccgtcagaactcactcggatctcaaggttggagagtactcagacctagaagtaaatgcatcttggacaaaagagagatggagccaagagtttactaagcaccaggttctcattatgacttgctatgtcgccttgactgttttgaaaaatggttacttatcactgtcagacattaaccttttggtgtttgatgagtgtcatcttgcgattctagaccacccctatcgtgaaattatgaagctctgtgagagttgtccatcatgccctcgaattttgggactaactgcttccattttaaatgggaaatgtgatccagaggaattggaagagaagattcagaaactggagcgaattctcaggagtgacgctgagacggcgactgacttggtggtcctggacagatacacttctcagccttgtgagattgtggtagactgtggaccgtttactgacagaagtgggctgtatgagagattgctgatggagttagaagcagcacttgattttatcaatgattgtaatgtagctgtacattcaaaggaaagagattctactttgatttccaaacaaatcctctcagactgccgtgcagtgttggttgttctgggaccctggtgtgcagataaagtagctgggatgatggtaagagaacttcagaagtacatcaaacatgagcaagaggagctacacaggaagttcctattgtttacagacaccttgttaaggaaaatacacgccctgtgtgaagagtacttctcacctgcctcacttgacctgaagtatgtaactcccaaagtcatgaaactgctcgagatcctgcgcaaatacaagccctatgagcgacagcagtttgaaagtgttgagtggtacaataacaggaatcaggataattacgtgtcctggagtgattccgaggatgacgatgatgacgaagaaattgaagaaaaagaaaagccagagacaaattttccttctccgtttaccaatattttatgtgggattatttttgtggaaagaagatacacagcagttgtcctaaacagattgataaaggaagctggcaaacaagatccagagctggcttatatcagcagcaactttataactggacacggcattggaaagaaccagcctcgcagcaaacagatggaggcggagttcagaaagcaggaggaggtacttaggaaatttcgagctcatgagaccaacctgctcattgcaacaagtgttgtagaagagggtgtggatattcccaagtgcaatttggtggttcgttttgatttgcctacagagtatcgatcatatgtccagtctaaaggacgggcacgagcgccgatctctaattacgtaatgttagcagatacagacaagataaagagctttgaagaagacctcaagacctacaaagctattgaaaagatcttaaggaacaagtgttcgaagtcagcggatggtgctgaggcggacgtccatgctggtgtagatgatgaggatgctttcccgccatatgtgctgaggcctgatgatgggggtcctcgagtcacaatcaacactgccattggacacatcaacagatattgtgctcgattaccaagtgatccgtttacgcatctagctcccaaatgtagaacccgagaattgcctgatggtacattttattcaactctttatcttccgattaactcacctcttcgagcctccattgttggtccaccgatggacagtgtacggttggctgaaagagtggtagctctcatttgctgtgaaaaactgcacaaaattggtgaactggatgaacacttgatgccagttgggaaagagactgttaaatatgaagaagagctggacttacatgacgaggaggagaccagtgttcccgggagaccagggtccacaaaacgacggcagtgctaccccaaagcaattccagagtgcttgagggagagttaccccaaacccgaccagccttgttacctgtatgtgataggaatggtcttaactacccccctccctgatgaactcaacttcaggcggcggaagctctacccacctgaggacaccacaagatgctttgggatcctgacagccaaacccatacctcagattcctcactttcctgtgtatacacgctctggagaggtcaccatatccatcgagctgaagaagtctggtttcacattgtctcaacaaatgctcgagctgattacaagattgcaccagtacatattctcacatattctccggcttgagaagcctgccctggagtttaaacctacaggcgcggagtcagcctactgtgttctacctcttaatgttgttaatgactccggcactttggacattgactttaaattcatggaggatattgagaagtcggaagctcggataggcattcctagcaccaagtattcaaaggaaacaccgtttgtctttaagttagaagattaccaagatgcagtgatcattccaagataccgcaattttgatcagcctcatcgattttatgtagctgatgtatatacggatcttaccccactcagtaaatttccttcccctgaatatgaaacttttgcagaatattataaaacaaagtataaccttgacctgaccaatctcaaccagccactgctggatgttgaccacacatcttccagactgaaccttttgacacctcggcatttgaatcagaaggggaaagcacttcctctaagcagtgctgagaagaggaaggccaaatgggaaagtctgcagaacaaacagatcctggttccggaactctgtgctatacatccaatcccagcatcactgtggagaaaagcggtttgtctccccagcatactgtatcgccttcactgccttctgactgcagaggaactaagagctcagacagccagcgatgctggtgtgggtgtcagatcacttcccgtggattttagataccctaacttagacttcgggtggaaaaaatctattgatagcaagtctttcatctcaagctgtaactcctcgttggctgagagtgataattactgtaagcacagcacaactgtagtccctgaacatgctgcacatcaaggtgctactagaccctccttagaaaaccatgaccaaatgtctgtgaactgcaaaaggttgcccgctgagtcacctgctaagctccaaagtgaggtttcaacggatcttacagcaattaatggtctttcttacaataaaaaccttgccaatggcagttatgatttagttaacagagacttttgccaaggaaatcagctgaattacttcaagcaggaaatacctgtacaaccaactacctcatatcccattcagaatttgtacaattatgagaaccagcccaagcccagcaatgaatgtcctctactgagtaatacgtaccttgatgggaacgctaacacatctacctcagatggaagccccgcggtgtccacgatgcctgccatgatgaatgctgttaaagcactcaaggacaggatggactctgagcagagcccttctgttgggtactcctcaaggactcttggccccaatccgggactgattctccaggctttgacactgtcaaatgccagtgatgggtttaacctggagcggcttgagatgcttggcgactcctttttaaagcatgctatcaccacatatctgttttgcacgtaccctgatgctcatgaaggccgcctttcatatatgagaagcaaaaaggtcagcaactgtaacctgtatcgccttggcaagaagaagggactgcccagccgcatggtggtgtcgatattcgatcctcctgtgaattggcttcctcctggttatgtggtaaaccaagacaaaagtaactcagagaagtgggaaaaggatgaaatgacaaaagactgcttgctggctaatggcaaactgggtgaggcctgtgaggaggaggaggacctgacgtggagggcccccaaggaggaggccgaggatgaggacgacttcctggagtatgaccaggagcatatccagtttatagacagcatgctcatgggctcgggcgccttcgtgaggaaaatctctctgtcgcccttctcagcctccgactctgcatacgaatggaagatgcccaagaaggcctccttgggcagtatgccgtttgcctcgggccttgaggattttgattacagctcttgggatgcgatgtgctatctggatcctagcaaagccgttgaagaggatgactttgtggtaggtttctggaatccatcagaagagaactgtggcgtcgacacagggaagcagtccatttcttacgacttgcacacggagcagtgcattgctgacaagagcatagcggactgtgtggaggccctgctgggctgctacttaaccagctgcggcgagagggctgctcagctcttcctctgttcgctggggctgaaggtgctcccagtaatcaaaaggaccagccgggagaaggccttggaccctgctcaggagaacggaagcagccaacaaaagagcctttcagggagctgtgcctctccagttggcccacgctcctctgcagggaaagacttggagtacggctgcctgaagatcccaccgaggtgcatgtttgaccatccagacgcagagaaaaccctcaaccacctgatatctgggtttgaaacttttgagaagaaaattaactatagattcaagaataaggcttatcttctgcaggcttttacacacgcctcctaccactacaacactatcactgattgttaccagcgcttagaattcctgggagatgcgattttggactacctcataaccaagcacctttatgaagacccacggcagcattctccaggggtcttgactgacttgcgctctgcccttgtcaataacaccatctttgcatcattggctgtgaagtatgactaccacaagtactttaaagctgtctctcctgagctctttcatgttattgatgactttgtgaagtttcagcttgagaagaacgaaatgcaaggaatggactctgagcttaggagatccgaggaggatgaggagaaagaagaggatatcgaagtccccaaggccatgggggacatttttgagtctcttgctggtgccatttatatggacagtgggatgtctctggaggtggtctggcaggtgtactatccgatgatgcagcctctaatagaaaagttttctgcaaatgtgcctcgttctccagtgagagaattgctcgagatggaaccagaaactgccaagtttagcccagcggagagaacttatgatggaaaggtcagagtcaccgtggaagtggtgggaaaggggaaatttaaaggtgttggccggagctacaggatcgccaagtctgctgcagcacgaagggccctccgcagcctcaaagctaaccagcctcaggttcctaacagctgaagcttccggactcacaggcggcaagacagcggaccagaaccacagctccacggcacaaggatggttcgggctgacacagaacaggaaaactgaaggcagaatttaaagtttgataatgagatagataacagaataaaacatgtaatatgtgtataaactctttggaactaattttggtttagtttttgcgcagacacaatcttgtcttctttacccaccctgctttgcttagtcacaggagtgctttaatcctggtgtttgttagcaaggttttaaaatcattttgacaggtttttggttttgtttgttttgacatttaatttctgtcgactttgcttggtgttagaggccatggagcttaacctctaacagtccctagaactgggtcgtcctgaccttgcggtcccttacactatacctgtcctgctgcggtgtcaagcccaacaaaaagggcagagtgtgcaaaggaagcccccgtgctgtggagggcagtggaggcggggccgggcggaccccgggtactattgcacggaatagtaagggacagattttgagtccgtgttggctgctggtgagtggaggccaggctttcagacgccacattgtgctgcagagaggattcttgccttaaaaggaaggacttgatagtttgcttttttattttgccttccccttattatgcattggatgaaatggggtggcagctcactagggcacttgcaatggaactattgcaggtaggatttctgttgtttgtactgaagacattctaaaacccagagtgagtatatagatttttaaagcatcattatagacaggatacatgactagatgcccggttttcctcagaacttcctgccaggtggtcccagtggcggaaatgcccggtcagctgctgttctgacgaaatgagcctgagatgcagtgtacctagacacgccacaggtctgtactaggcagtgttgccagagtcaccaaagctgtgctctctttcctatggggtcagcattgtggcagactgccaggaccaacccgtctggggtagaagtcaccagtttagataaactaggtcattagttcatctcagactcagaaagtaaagctgtttaggcatttgctgaaggtctcgcctgggcttgtgggcccctcttcacttacacacctcagtccctccctttgcctgcattttcccgttggtgctacgtttatgtatcatgcttgaagtttaattttttgcactgtgactataatacctcttaatttacatttaaaaatgctgtatgggttgcccgtgccctcccaccgacacacaggagagctttgctgcaatttgccctcttacgcttgaagtctcagcaggctgagccagggtagctcgcgttccccacacatctcctgaggctgatgcttcatgtaatgctgcacttaggttcaagtcacccgcccgagagagagagagagagagagagagagagagagagagagagagagatgctgtgtgtgtgcaagcaagcactcgggctgtcaggaaccccatggggagggagagtgagcgtccccaaggatcacgtgctgttgctgttgcccatctgcaaagcaggatctgagttattgtttggtgaaattaatttggctgactcatcttaagtatttaattctaacgtgcgattgtagttgtctgtttgacttagaacatccctctcccccatgcgcgcgcacacacacacacaaaccttaatgcaagacatcctagcatgtgcaaaccaggtgatcttttgaatgttgtagctataatttgtatagtcagatagtaaggtggggtgggattgtgggaggtggagtttgttgtcttatcacagtccttggggatttataaactacctgattctctgcattgtgattcgtgcatttatgtcctgggatggatgctctggcatcctggcaccaaaagcagatggtttcacacttggattcctggtggaagaagaaaatgcctcagtatattttgtaacataaggagagtattttttgttaatattgaaaagttcagaattggatatgcttcgttcatccattctcagggctcaaatgttttgccactattcagaatgttgtatcaatagcaaacttgggctgtttttctttttctttcttccttctttttttgattgctgttagataaaagtacctgataactagtttgaatcatactgtgatttacccagagaccaagtccgaaaaatcaaaagtatgagttagacgcgcgatcaggagtgcgtttgtttgtgttgggggttgtccccaggttcgctggttgaaagactttgcattttagagaaaaagttgttctgccccctccctcgatgactccatgagcctcctccactggtgtagcaggaggaggaggaggagacagccagcgcccatgaggaggaagcagctgcacagatcctgtggctgcatgcgagctcggcgggcactgcactgttgggccgtcccctgtggcccttttgcaatcccaggcaggcagagtgagtgaggccctgtcctctgttgggttgtgtctgtgtggcacaggctgggcagtgctccggcactcacgcttaacccagaggggaggccatgcttgggcctcaccgcggttccgacttgagaacattctgggatgcctgcagcttgtcacacaattgcacatttgaactgttattatttttgtctgtgacattttcttttatctgtaaagttgaaagcatggcagagatttgttgactgaacacttaattatgctttttaaatgttttggttacgtttcttctttttttcgttcatattatgtgaaatgattaaatttagaataacctctaacactcctataattatcgtttaaaataccgatgtttttattttctcgacagtagtccgtcctcagaaagatgcagacaccctgtgtaattagcagggacttgaggctgcttcggttctgccgctcagttcccgtgtacagtcaccagcagccaccagacagtggctgagagcgtggagggagaccagtcaaaaacccacaaccacccgctttgctcgtggggcctgttgatttaaaactactccttgctcggtgaaggcacctagtcctcctgcacaaacttaactgctggcaatccgaggccttgcacctttatttcacagaccctgagctgagcctacagccgcatccttagccacctcgggctctgtgtgtctcctctgtgcttaacaacgcccaagaacccatagcttcccatctgctgattgcttctctggaatttaattatttctcaataaattctgtattttatattgtacgtgagacattttaaagatatgtttgagaccaaggatagttaaaatgactgtaaaggtagtgtgtctgccatggagacggagtgctgtccactggcagggaaaatgatcacctcaaactaatttgcagttattatttgactcattattttcttgtctgtctttctttgataaacaaccacagacagtttaaatgtcatacatggcctcagaatttgccttggctgctggttccatagcagaagcactgaggtgaagggaagaacatatctgccttttgtgctgtaaataaagggaaatgactaacggagtcaggctgagtgttctgccgacaagaaacggaatcacatcacactctgcagccttctaaagaaacctgtatccatgcacacagctatcactgcagagctgtgccagcacctatggaccacagttacctttagcttttccgttttgttttgtttttctttttctttttaataattgtagtcaataaagttattgtctgttca

SEQ ID NO: 15 - protein, 1906 aa, Homo sapiens
MAGLQLMTPASSPMGPFFGLPWQQEAIHDNIYTPRKYQVELLEAALDHNTIVCLNTGSGKTFIAVLLTKELAHQIRGDLNPHAKRTVFLVNSANQVAQQVSAVRTHSDLKVGEYSDLEVNASWTKERWSQEFTKHQVLIMTCYVALTVLKNGYLSLSDINLLVFDECHLAILDHPYREIMKLCESCPSCPRILGLTASILNGKCDPEELEEKIQKLERILRSDAETATDLVVLDRYTSQPCEIVVDCGPFTDRSGLYERLLMELEAALDFINDCNVAVHSKERDSTLISKQILSDCRAVLVVLGPWCADKVAGMMVRELQKYIKHEQEELHRKFLLFTDTLLRKIHALCEEYFSPASLDLKYVTPKVMKLLEILRKYKPYERQQFESVEWYNNRNQDNYVSWSDSEDDDDDEEIEEKEKPETNFPSPFTNILCGIIFVERRYTAVVLNRLIKEAGKQDPELAYISSNFITGHGIGKNQPRSKQMEAEFRKQEEVLRKFRAHETNLLIATSVVEEGVDIPKCNLVVRFDLPTEYRSYVQSKGRARAPISNYVMLADTDKIKSFEEDLKTYKAIEKILRNKCSKSADGAEADVHAGVDDEDAFPPYVLRPDDGGPRVTINTAIGHINRYCARLPSDPFTHLAPKCRTRELPDGTFYSTLYLPINSPLRASIVGPPMDSVRLAERVVALICCEKLHKIGELDEHLMPVGKETVKYEEELDLHDEEETSVPGRPGSTKRRQCYPKAIPECLRESYPKPDQPCYLYVIGMVLTTPLPDELNFRRRKLYPPEDTTRCFGILTAKPIPQIPHFPVYTRSGEVTISIELKKSGFTLSQQMLELITRLHQYIFSHILRLEKPALEFKPTGAESAYCVLPLNVVNDSGTLDIDFKFMEDIEKSEARIGIPSTKYSKETPFVFKLEDYQDAVIIPRYRNFDQPHRFYVADVYTDLTPLSKFPSPEYETFAEYYKTKYNLDLTNLNQPLLDVDHTSSRLNLLTPRHLNQKGKALPLSSAEKRKAKWESLQNKQILVPELCAIHPIPASLWRKAVCLPSILYRLHCLLTAEELRAQTASDAGVGVRSLPVDFRYPNLDFGWKKSIDSKSFISSCNSSLAESDNYCKHSTTVVPEHAAHQGATRPSLENHDQMSVNCKRLPAESPAKLQSEVSTDLTAINGLSYNKNLANGSYDLVNRDFCQGNQLNYFKQEIPVQPTTSYPIQNLYNYENQPKPSNECPLLSNTYLDGNANTSTSDGSPAVSTMPAMMNAVKALKDRMDSEQSPSVGYSSRTLGPNPGLILQALTLSNASDGFNLERLEMLGDSFLKHAITTYLFCTYPDAHEGRLSYMRSKKVSNCNLYRLGKKKGLPSRMVVSIFDPPVNWLPPGYVVNQDKSNSEKWEKDEMTKDCLLANGKLGEACEEEEDLTWRAPKEEAEDEDDFLEYDQEHIQFIDSMLMGSGAFVRKISLSPFSASDSAYEWKMPKKASLGSMPFASGLEDFDYSSWDAMCYLDPSKAVEEDDFVVGFWNPSEENCGVDTGKQSISYDLHTEQCIADKSIADCVEALLGCYLTSCGERAAQLFLCSLGLKVLPVIKRTSREKALDPAQENGSSQQKSLSGSCASPVGPRSSAGKDLEYGCLKIPPRCMFDHPDAEKTLNHLISGFETFEKKINYRFKNKAYLLQAFTHASYHYNTITDCYQRLEFLGDAILDYLITKHLYEDPRQHSPGVLTDLRSALVNNTIFASLAVKYDYHKYFKAVSPELFHVIDDFVKFQLEKNEMQGMDSELRRSEEDEEKEEDIEVPKAMGDIFESLAGAIYMDSGMSLEVVWQVYYPMMQPLIEKFSANVPRSPVRELLEMEPETAKFSPAERTYDGKVRVTVEVVGKGKFKGVGRSYRIAKSAAARRALRSLKANQPQVPNS

SEQ ID NO: 16 - DNA, 10323 bp, Homo sapiens
  CDS (239)..(6007)
cggaggcgcggcgcaggctgctgcaggcccaggtgaatggagtaacctgacagcggggacgaggcgacggcgagcgcgaggaaatggcggcgggggcggcggcgccgggcggctccgggaggcctgggctgtgacgcgcgcgccggagcggggtccgatggttctcgaaggcccgcggcgccccgtgctgcagtaagctgtgctagaacaaaaatgcaatgaaagaaacactggatgaatgaaaagccctgctttgcaacccctcagcatggcaggcctgcagctcatgacccctgcttcctcaccaatgggtcctttctttggactgccatggcaacaagaagcaattcatgataacatttatacgccaagaaaatatcaggttgaactgcttgaagcagctctggatcataataccatcgtctgtttaaacactggctcagggaagacatttattgcagtactactcactaaagagctgtcctatcagatcaggggagacttcagcagaaatggaaaaaggacggtgttcttggtcaactctgcaaaccaggttgctcaacaagtgtcagctgtcagaactcattcagatctcaaggttggggaatactcaaacctagaagtaaatgcatcttggacaaaagagagatggaaccaagagtttactaagcaccaggttctcattatgacttgctatgtcgccttgaatgttttgaaaaatggttacttatcactgtcagacattaaccttttggtgtttgatgagtgtcatcttgcaatcctagaccacccctatcgagaaattatgaagctctgtgaaaattgtccatcatgtcctcgcattttgggactaactgcttccattttaaatgggaaatgtgatccagaggaattggaagaaaagattcagaaactagagaaaattcttaagagtaatgctgaaactgcaactgacctggtggtcttagacaggtatacttctcagccatgtgagattgtggtggattgtggaccatttactgacagaagtgggctttatgaaagactgctgatggaattagaagaagcacttaattttatcaatgattgtaatatatctgtacattcaaaagaaagagattctactttaatttcgaaacagatactatcagactgtcgtgccgtattggtagttctgggaccctggtgtgcagataaagtagctggaatgatggtaagagaactacagaaatacatcaaacatgagcaagaggagctgcacaggaaatttttattgtttacagacactttcctaaggaaaatacatgcactatgtgaagagcacttctcacctgcctcacttgacctgaaatttgtaactcctaaagtaatcaaactgctcgaaatcttacgcaaatataaaccatatgagcgacagcagtttgaaagcgttgagtggtataataatagaaatcaggataattatgtgtcatggagtgattctgaggatgatgatgaggatgaagaaattgaagaaaaagagaagccagagacaaattttccttctccttttaccaacattttgtgcggaattatttttgtggaaagaagatacacagcagttgtcttaaacagattgataaaggaagctggcaaacaagatccagagctggcttatatcagtagcaatttcataactggacatggcattgggaagaatcagcctcgcaacaaacagatggaagcagaattcagaaaacaggaagaggtacttaggaaatttcgagcacatgagaccaacctgcttattgcaacaagtattgtagaagagggtgttgatataccaaaatgcaacttggtggttcgttttgatttgcccacagaatatcgatcctatgttcaatctaaaggaagagcaagggcacccatctctaattatataatgttagcggatacagacaaaataaaaagttttgaagaagaccttaaaacctacaaagctattgaaaagatcttgagaaacaagtgttccaagtcggttgatactggtgagactgacattgatcctgtcatggatgatgatgacgttttcccaccatatgtgttgaggcctgacgatggtggtccacgagtcacaatcaacacggccattggacacatcaatagatactgtgctagattaccaagtgatccgtttactcatctagctcctaaatgcagaacccgagagttgcctgatggtacattttattcaactctttatctgccaattaactcacctcttcgagcctccattgttggtccaccaatgagctgtgtacgattggctgaaagagttgtagctctcatttgctgtgagaaactgcacaaaattggcgaactggatgaccatttgatgccagttgggaaagagactgttaaatatgaagaggagcttgatttgcatgatgaagaagagaccagtgttccaggaagaccaggttccacgaaacgaaggcagtgctacccaaaagcaattccagagtgtttgagggatagttatcccagacctgatcagccctgttacctgtatgtgataggaatggttttaactacacctttacctgatgaactcaactttagaaggcggaagctctatcctcctgaagataccacaagatgctttggaatactgacggccaaacccatacctcagattccacactttcctgtgtacacacgctctggagaggttaccatatccattgagttgaagaagtctggtttcatgttgtctctacaaatgcttgagttgattacaagacttcaccagtatatattctcacatattcttcggcttgaaaaacctgcactagaatttaaacctacagacgctgattcagcatactgtgttctacctcttaatgttgttaatgactccagcactttggatattgactttaaattcatggaagatattgagaagtctgaagctcgcataggcattcccagtacaaagtatacaaaagaaacaccctttgtttttaaattagaagattaccaagatgccgttatcattccaagatatcgcaattttgatcagcctcatcgattttatgtagctgatgtgtacactgatcttaccccactcagtaaatttccttcccctgagtatgaaacttttgcagaatattataaaacaaagtacaaccttgacctaaccaatctcaaccagccactgctggatgtggaccacacatcttcaagacttaatcttttgacacctcgacatttgaatcagaaggggaaagcgcttcctttaagcagtgctgagaagaggaaagccaaatgggaaagtctgcagaataaacagatactggttccagaactctgtgctatacatccaattccagcatcactgtggagaaaagctgtttgtctccccagcatactttatcgccttcactgccttttgactgcagaggagctaagagcccagactgccagcgatgctggcgtgggagtcagatcacttcctgcggattttagataccctaacttagacttcgggtggaaaaaatctattgacagcaaatctttcatctcaatttctaactcctcttcagctgaaaatgataattactgtaagcacagcacaattgtccctgaaaatgctgcacatcaaggtgctaatagaacctcctctctagaaaatcatgaccaaatgtctgtgaactgcagaacgttgctcagcgagtcccctggtaagctccacgttgaagtttcagcagatcttacagcaattaatggtctttcttacaatcaaaatctcgccaatggcagttatgatttagctaacagagacttttgccaaggaaatcagctaaattactacaagcaggaaatacccgtgcaaccaactacctcatattccattcagaatttatacagttacgagaaccagccccagcccagcgatgaatgtactctcctgagtaataaataccttgatggaaatgctaacaaatctacctcagatggaagtcctgtgatggccgtaatgcctggtacgacagacactattcaagtgctcaagggcaggatggattctgagcagagcccttctattgggtactcctcaaggactcttggccccaatcctggacttattcttcaggctttgactctgtcaaacgctagtgatggatttaacctggagcggcttgaaatgcttggcgactcctttttaaagcatgccatcaccacatatctattttgcacttaccctgatgcgcatgagggccgcctttcatatatgagaagcaaaaaggtcagcaactgtaatctgtatcgccttggaaaaaagaagggactacccagccgcatggtggtgtcaatatttgatccccctgtgaattggcttcctcctggttatgtagtaaatcaagacaaaagcaacacagataaatgggaaaaagatgaaatgacaaaagactgcatgctggcgaatggcaaactggatgaggattacgaggaggaggatgaggaggaggagagcctgatgtggagggctccgaaggaagaggctgactatgaagatgatttcctggagtatgatcaggaacatatcagatttatagataatatgttaatggggtcaggagcttttgtaaagaaaatctctctttctcctttttcaaccactgattctgcatatgaatggaaaatgcccaaaaaatcctccttaggtagtatgccattttcatcagattttgaggattttgactacagctcttgggatgcaatgtgctatctggatcctagcaaagctgttgaagaagatgactttgtggtggggttctggaatccatcagaagaaaactgtggtgttgacacgggaaagcagtccatttcttacgacttgcacactgagcagtgtattgctgacaaaagcatagcggactgtgtggaagccctgctgggctgctatttaaccagctgtggggagagggctgctcagcttttcctctgttcactggggctgaaggtgctcccggtaattaaaaggactgatcgggaaaaggccctgtgccctactcgggagaatttcaacagccaacaaaagaacctttcagtgagctgtgctgctgcttctgtggccagttcacgctcttctgtattgaaagactcggaatatggttgtttgaagattccaccaagatgtatgtttgatcatccagatgcagataaaacactgaatcaccttatatcggggtttgaaaattttgaaaagaaaatcaactacagattcaagaataaggcttaccttctccaggcttttacacatgcctcctaccactacaatactatcactgattgttaccagcgcttagaattcctgggagatgcgattttggactacctcataaccaagcacctttatgaagacccgcggcagcactccccgggggtcctgacagacctgcggtctgccctggtcaacaacaccatctttgcatcgctggctgtaaagtacgactaccacaagtacttcaaagctgtctctcctgagctcttccatgtcattgatgactttgtgcagtttcagcttgagaagaatgaaatgcaaggaatggattctgagcttaggagatctgaggaggatgaagagaaagaagaggatattgaagttccaaaggccatgggggatatttttgagtcgcttgctggtgccatttacatggatagtgggatgtcactggagacagtctggcaggtgtactatcccatgatgcggccactaatagaaaagttttctgcaaatgtaccccgttcccctgtgcgagaattgcttgaaatggaaccagaaactgccaaatttagcccggctgagagaacttacgacgggaaggtcagagtcactgtggaagtagtaggaaaggggaaatttaaaggtgttggtcgaagttacaggattgccaaatctgcagcagcaagaagagccctccgaagcctcaaagctaatcaacctcaggttcccaatagctgaaaccgctttttaaaattcaaaacaagaaacaaaacaaaaaaaattaaggggaaaattatttaaatcggaaaggaagacttaaagttgttagtgagtggaatgaattgaaggcagaatttaaagtttggttgataacaggatagataacagaataaaacatttaacatatgtataaaattttggaactaattgtagttttagttttttgcgcaaacacaatcttatcttctttcctcacttctgctttgtttaaatcacaagagtgctttaatgatgacatttagcaagtgctcaaaataattgacaggttttgtttttttttttttgagtttatgtcagctttgcttagtgttagaaggccatggagcttaaacctccagcagtccctaggatgatgtagattcttctccatctctccgtgtgtgcagtagtgccagtcctgcagtagttgataagctgaatagaaagataaggttttcgagaggagaagtgcgccaatgttgtcttttctttccacgttatactgtgtaaggtgatgttcccggtcgctgttgcacctgatagtaagggacagatttttaatgaacattggctggcatgttggtgaatcacattttagttttctgatgccacatagtcttgcataaaaaagggttcttgccttaaaagtgaaaccttcatggatagtctttaatctctgatctttttggaacaaactgttttacattcctttcattttattatgcattagacgttgagacagcgtgatacttacaactcactagtatagttgtaacttattacaggatcatactaaaatttctgtcatatgtatactgaagacattttaaaaaccagaatatgtagtctacggatattttttatcataaaaatgatctttggctaaacaccccattttactaaagtcctcctgccaggtagttcccactgatggaaatgtttatggcaaataattttgccttctaggctgttgctctaacaaaataaaccttagacatatcacacctaaaatatgctgcagattttataattgattggttacttatttaagaagcaaaacacagcacctttacccttagtctcctcacataaatttcttactatacttttcataatgttgcatgcatatttcacctaccaaagctgtgctgttaatgccgtgaaagtttaacgtttgcgataaactgccgtaattttgatacatctgtgatttaggtcattaatttagataaactagctcattatttccatctttggaaaaggaaaaaaaaaaaaacttctttaggcatttgcctaagtttctttaattagacttgtaggcactcttcacttaaatacctcagttcttcttttcttttgcatgcatttttcccctgtttggtgctatgtttatgtattatgcttgaaattttaatttttttttttttgcactgtaactataatacctcttaatttacctttttaaaagctgtgggtcagtcttgcactcccatcaacataccagtagaggtttgctgcaatttgccccgttaattatgcttgaagtttaagaaagctgagcagaggtgtctcatatttcccagcacatgattctgaacttgatgcttcgtggaatgctgcatttatatgtaagtgacatttgaatactgtccttcctgctttatctgcatcatccacccacagagaaatgcctctgtgcgagtgcaccgacagaaaactgtcagctctgctttctaaggaaccctgagtgaggggggtattaagcttctccagtgttttttgttgtctccaatcttaaacttaaattgagatctaaattattaaacgagtttttgagcaaattaggtgacttgttttaaaaatatttaattccgatttggaaccttagatgtctatttgattttttaaaaaaccttaatgtaagatatgaccagttaaaacaaagcaattcttgaattatataactgtaaaagtgtgcagttaacaaggctggatgtgaattttattctgagggtgatttgtgatcaagtttaatcacaaatctcttaatatttataaactacctgatgccaggagcttagggctttgcattgtgtctaatacattgatcccagtgttacgggattctcttgattcctggcaccaaaatcagattgttttcacagttatgattcccagtgggagaaaaatgcctcaatatatttgtaaccttaagaagagtatttttttgttaatactaagatgttcaaacttagacatgattaggtcatacattctcaggggttcaaatttccttctaccattcaaatgttttatcaacagcaaacttcagccgtttcactttttgttggagaaaaatagtagattttaatttgactcacagtttgaagcattctgtgatcccctggttactgagttaaaaaataaaaaagtacgagttagacatatgaaatggttatgaacgcttttgtgctgctgatttttaatgctgtaaagttttcctgtgtttagcttgttgaaatgttttgcatctgtcaattaaggaaaaaaaaaatcactctatgttgccccactttagagccctgtgtgccaccctgtgttcctgtgattgcaatgtgagaccgaatgtaatatggaaaacctaccagtggggtgtggttgtgccctgagcacgtgtgtaaaggactggggaggcgtgtcttgaaaaagcaactgcagaaattccttatgatgattgtgtgcaagttagttaacatgaaccttcatttgtaaattttttaaaatttcttttataatatgctttccgcagtcctaactatgctgcgttttataatagctttttcccttctgttctgttcatgtagcacagataagcattgcacttggtaccatgctttacctcatttcaagaaaatatgcttaacagagaggaaaaaaatgtggtttggccttgctgctgttttgatttatggaatttgaaaaagataattataatgcctgcaatgtgtcatatactcgcacaacttaaataggtcatttttgtctgtggcatttttactgtttgtgaaagtatgaaacagatttgttaactgaactcttaattatgtttttaaaatgtttgttatatttcttttcttttttcttttatattacgtgaagtgatgaaatttagaatgacctctaacactcctgtaattgtcttttaaaatactgatatttttatttgttaataatactttgccctcagaaagattctgataccctgccttgacaacatgaaacttgaggctgctttggttcatgaatccaggtgttcccccggcagtcggcttcttcagtcgctccctggaggcaggtgggcactgcagaggatcactggaatccagatcgagcgcagttcatgcacaaggccccgttgatttaaaatattggatcttgctctgttagggtgtctaatccctttacacaagattgaagccaccaaactgagaccttgatacctttttttaactgcatctgaaattatgttaagagtctttaacccatttgcattatctgcagaagagaaactcatgtcatgtttattacctatatggttgttttaattacatttgaataattatatttttccaaccactgattacttttcaggaatttaattatttccagataaatttctttattttatattgtacatgaaaagttttaaagatatgtttaagaccaagactattaaaatgatttttaaagttgttggagacgccaatagcaatatctaggaaatttgcattgagaccattgtattttccactagcagtgaaaatgatttttcacaactaacttgtaaatatattttaatcattacttctttttttctagtccatttttatttggacatcaaccacagacaatttaaattttatagatgcactaagaattcactgcagcagcaggttacatagcaaaaatgcaaaggtgaacaggaagtaaatttctggcttttctgctgtaaatagtgaaggaaaattactaaaatcaagtaaaactaatgcatattatttgattgacaataaaatatttaccatcacatgctgcagctgttttttaaggaacatgatgtcattcattcatacagtaatcatgctgcagaaatttgcagtctgcaccttatggatcacaattacctttagttgttttttttgtaataattgtagccaagtaaatctccaataaagttatcgtctgttcaaaaaaaaaaaaaaaaaaaaaaaaaaaaaaaaaaaaaaaaaaaaaaa

SEQ ID NO: 17 - protein, 1922 aa, Homo sapiens
MKSPALQPLSMAGLQLMTPASSPMGPFFGLPWQQEAIHDNIYTPRKYQVELLEAALDHNTIVCLNTGSGKTFIAVLLTKELSYQIRGDFSRNGKRTVFLVNSANQVAQQVSAVRTHSDLKVGEYSNLEVNASWTKERWNQEFTKHQVLIMTCYVALNVLKNGYLSLSDINLLVFDECHLAILDHPYREIMKLCENCPSCPRILGLTASILNGKCDPEELEEKIQKLEKILKSNAETATDLVVLDRYTSQPCEIVVDCGPFTDRSGLYERLLMELEEALNFINDCNISVHSKERDSTLISKQILSDCRAVLVVLGPWCADKVAGMMVRELQKYIKHEQEELHRKFLLFTDTFLRKIHALCEEHFSPASLDLKFVTPKVIKLLEILRKYKPYERQQFESVEWYNNRNQDNYVSWSDSEDDDEDEEIEEKEKPETNFPSPFTNILCGIIFVERRYTAVVLNRLIKEAGKQDPELAYISSNFITGHGIGKNQPRNKQMEAEFRKQEEVLRKFRAHETNLLIATSIVEEGVDIPKCNLVVRFDLPTEYRSYVQSKGRARAPISNYIMLADTDKIKSFEEDLKTYKAIEKILRNKCSKSVDTGETDIDPVMDDDDVFPPYVLRPDDGGPRVTINTAIGHINRYCARLPSDPFTHLAPKCRTRELPDGTFYSTLYLPINSPLRASIVGPPMSCVRLAERVVALICCEKLHKIGELDDHLMPVGKETVKYEEELDLHDEEETSVPGRPGSTKRRQCYPKAIPECLRDSYPRPDQPCYLYVIGMVLTTPLPDELNFRRRKLYPPEDTTRCFGILTAKPIPQIPHFPVYTRSGEVTISIELKKSGFMLSLQMLELITRLHQYIFSHILRLEKPALEFKPTDADSAYCVLPLNVVNDSSTLDIDFKFMEDIEKSEARIGIPSTKYTKETPFVFKLEDYQDAVIIPRYRNFDQPHRFYVADVYTDLTPLSKFPSPEYETFAEYYKTKYNLDLTNLNQPLLDVDHTSSRLNLLTPRHLNQKGKALPLSSAEKRKAKWESLQNKQILVPELCAIHPIPASLWRKAVCLPSILYRLHCLLTAEELRAQTASDAGVGVRSLPADFRYPNLDFGWKKSIDSKSFISISNSSSAENDNYCKHSTIVPENAAHQGANRTSSLENHDQMSVNCRTLLSESPGKLHVEVSADLTAINGLSYNQNLANGSYDLANRDFCQGNQLNYYKQEIPVQPTTSYSIQNLYSYENQPQPSDECTLLSNKYLDGNANKSTSDGSPVMAVMPGTTDTIQVLKGRMDSEQSPSIGYSSRTLGPNPGLILQALTLSNASDGFNLERLEMLGDSFLKHAITTYLFCTYPDAHEGRLSYMRSKKVSNCNLYRLGKKKGLPSRMVVSIFDPPVNWLPPGYVVNQDKSNTDKWEKDEMTKDCMLANGKLDEDYEEEDEEEESLMWRAPKEEADYEDDFLEYDQEHIRFIDNMLMGSGAFVKKISLSPFSTTDSAYEWKMPKKSSLGSMPFSSDFEDFDYSSWDAMCYLDPSKAVEEDDFVVGFWNPSEENCGVDTGKQSISYDLHTEQCIADKSIADCVEALLGCYLTSCGERAAQLFLCSLGLKVLPVIKRTDREKALCPTRENFNSQQKNLSVSCAAASVASSRSSVLKDSEYGCLKIPPRCMFDHPDADKTLNHLISGFENFEKKINYRFKNKAYLLQAFTHASYHYNTITDCYQRLEFLGDAILDYLITKHLYEDPRQHSPGVLTDLRSALVNNTIFASLAVKYDYHKYFKAVSPELFHVIDDFVQFQLEKNEMQGMDSELRRSEEDEEKEEDIEVPKAMGDIFESLAGAIYMDSGMSLETVWQVYYPMMRPLIEKFSANVPRSPVRELLEMEPETAKFSPAERTYDGKVRVTVEVVGKGKFKGVGRSYRIAKSAAARRALRSLKANQPQVPNS

SEQ ID NO: 18 - DNA, 24 bp, Mus musculus
ccttgcgtggtcagcattagcatt

SEQ ID NO: 19 - DNA, 24 bp, Mus musculus
ttctcctcatcctcctcggatctc

SEQ ID NO: 20 - DNA, 3912 bp, Artificial Sequence
  Artifically constructed nucleotides 1906-5408 of the human DICER1 ORF with a 5′ ATG added
  CDS (1)..(3912)
atgggtggtccacgagtcacaatcaacacggccattggacacatcaatagatactgtgctagattaccaagtgatccgtttactcatctagctcctaaatgcagaacccgagagttgcctgatggtacattttattcaactctttatctgccaattaactcacctcttcgagcctccattgttggtccaccaatgagctgtgtacgattggctgaaagagttgtcgctctcatttgctgtgagaaactgcacaaaattggcgaactggatgaccatttgatgccagttgggaaagagactgttaaatatgaagaggagcttgatttgcatgatgaagaagagaccagtgttccaggaagaccaggttccacgaaacgaaggcagtgctacccaaaagcaattccagagtgtttgagggatagttatcccagacctgatcagccctgttacctgtatgtgataggaatggttttaactacacctttacctgatgaactcaactttagaaggcggaagctctatcctcctgaagataccacaagatgctttggaatactgacggccaaacccatacctcagattccacactttcctgtgtacacacgctctggagaggttaccatatccattgagttgaagaagtctggtttcatgttgtctctacaaatgcttgagttgattacaagacttcaccagtatatattctcacatattcttcggcttgaaaaacctgcactagaatttaaacctacagacgctgattcagcatactgtgttctacctcttaatgttgttaatgactccagcactttggatattgactttaaattcatggaagatattgagaagtctgaagctcgcataggcattcccagtacaaagtatacaaaagaaacaccctttgtttttaaattagaagattaccaagatgccgttatcattccaagatatcgcaattttgatcagcctcatcgattttatgtagctgatgtgtacactgatcttaccccactcagtaaatttccttcccctgagtatgaaacttttgcagaatattataaaacaaagtacaaccttgacctaaccaatctcaaccagccactgctggatgtggaccacacatcttcaagacttaatcttttgacacctcgacatttgaatcagaaggggaaagcgcttcctttaagcagtgctgagaagaggaaagccaaatgggaaagtctgcagaataaacagatactggttccagaactctgtgctatacatccaattccagcatcactgtggagaaaagctgtttgtctccccagcatactttatcgccttcactgccttttgactgcagaggagctaagagcccagactgccagcgatgctggcgtgggagtcagatcacttcctgcggattttagataccctaacttagacttcgggtggaaaaaatctattgacagcaaatctttcatctcaatttctaactcctcttcagctgaaaatgataattactgtaagcacagcacaattgtccctgaaaatgctgcacatcaaggtgctaatagaacctcctctctagaaaatcatgaccaaatgtctgtgaactgcagaacgttgctcagcgagtcccctggtaagctccacgttgaagtttcagcagatcttacagcaattaatggtctttcttacaatcaaaatctcgccaatggcagttatgatttagctaacagagacttttgccaaggaaatcagctaaattactacaagcaggaaatacccgtgcaaccaactacctcatattccattcagaatttatacagttacgagaaccagccccagcccagcgatgaatgtactctcctgagtaataaataccttgatggaaatgctaacaaatctacctcagatggaagtcctgtgatggccgtaatgcctggtacgacagacactattcaagtgctcaagggcaggatggattctgagcagagcccttctattgggtactcctcaaggactcttggccccaatcctggacttattcttcaggctttgactctgtcaaacgctagtgatggatttaacctggagcggcttgaaatgcttggcgactcctttttaaagcatgccatcaccacatatctattttgcacttaccctgatgcgcatgagggccgcctttcatatatgagaagcaaaaaggtcagcaactgtaatctgtatcgccttggaaaaaagaagggactacccagccgcatggtggtgtcaatatttgatccccctgtgaattggcttcctcctggttatgtagtaaatcaagacaaaagcaacacagataaatgggaaaaagatgaaatgacaaaagactgcatgctggcgaatggcaaactggatgaggattacgaggaggaggatgaggaggaggagagcctgatgtggagggctccgaaggaagaggctgactatgaagatgatttcctggagtatgatcaggaacatatcagatttatagataatatgttaatggggtcaggagcttttgtaaagaaaatctctctttctcctttttcaaccactgattctgcatatgaatggaaaatgcccaaaaaatcctccttaggtagtatgccattttcatcagattttgaggattttgactacagctcttgggatgcaatgtgctatctggatcctagcaaagctgttgaagaagatgactttgtggtggggttctggaatccatcagaagaaaactgtggtgttgacacgggaaagcagtccatttcttacgacttgcacactgagcagtgtattgctgacaaaagcatagcggactgtgtggaagccctgctgggctgctatttaaccagctgtggggagagggctgctcagcttttcctctgttcactggggctgaaggtgctcccggtaattaaaaggactgatcgggaaaaggccctgtgccctactcgggagaatttcaacagccaacaaaagaacctttcagtgagctgtgctgctgcttctgtggccagttcacgctcttctgtattgaaagactcggaatatggttgtttgaagattccaccaagatgtatgtttgatcatccagatgcagataaaacactgaatcaccttatatcggggtttgaaaattttgaaaagaaaatcaactacagattcaagaataaggcttaccttctccaggcttttacacatgcctcctaccactacaatactatcactgattgttaccagcgcttagaattcctgggagatgcgattttggactacctcataaccaagcacctttatgaagacccgcggcagcactccccgggggtcctgacagacctgcggtctgccctggtcaacaacaccatctttgcatcgctggctgtaaagtacgactaccacaagtacttcaaagctgtctctcctgagctcttccatgtcattgatgactttgtgcagtttcagcttgagaagaatgaaatgcaaggaatggattctgagcttaggagatctgaggaggatgaagagaaagaagaggatattgaagttccaaaggccatgggggatatttttgagtcgcttgctggtgccatttacatggatagtgggatgtcactggagacagtctggcaggtgtactatcccatgatgcggccactaatagaaaagttttctgcaaatgtaccccgttcccctgtgcgagaattgcttgaaatggaaccagaaactgccaaatttagcccggctgagagaacttacgacgggaaggtcagagtcactgtggaagtagtaggaaaggggaaatttaaaggtgttggtcgaagttacaggattgccaaatctgcagcagcaagaagagccctccgaagcctcaaagctaatcaacctcaggttcccaatagctga

SEQ ID NO: 21 - protein, 1303 aa, Artificial Sequence
  Synthetic Construct
MGGPRVTINTAIGHINRYCARLPSDPFTHLAPKCRTRELPDGTFYSTLYLPINSPLRASIVGPPMSCVRLAERVVALICCEKLHKIGELDDHLMPVGKETVKYEEELDLHDEEETSVPGRPGSTKRRQCYPKAIPECLRDSYPRPDQPCYLYVIGMVLTTPLPDELNFRRRKLYPPEDTTRCFGILTAKPIPQIPHFPVYTRSGEVTISIELKKSGFMLSLQMLELITRLHQYIFSHILRLEKPALEFKPTDADSAYCVLPLNVVNDSSTLDIDFKFMEDIEKSEARIGIPSTKYTKETPFVFKLEDYQDAVIIPRYRNFDQPHRFYVADVYTDLTPLSKFPSPEYETFAEYYKTKYNLDLTNLNQPLLDVDHTSSRLNLLTPRHLNQKGKALPLSSAEKRKAKWESLQNKQILVPELCAIHPIPASLWRKAVCLPSILYRLHCLLTAEELRAQTASDAGVGVRSLPADFRYPNLDFGWKKSIDSKSFISISNSSSAENDNYCKHSTIVPENAAHQGANRTSSLENHDQMSVNCRTLLSESPGKLHVEVSADLTAINGLSYNQNLANGSYDLANRDFCQGNQLNYYKQEIPVQPTTSYSIQNLYSYENQPQPSDECTLLSNKYLDGNANKSTSDGSPVMAVMPGTTDTIQVLKGRMDSEQSPSIGYSSRTLGPNPGLILQALTLSNASDGFNLERLEMLGDSFLKHAITTYLFCTYPDAHEGRLSYMRSKKVSNCNLYRLGKKKGLPSRMVVSIFDPPVNWLPPGYVVNQDKSNTDKWEKDEMTKDCMLANGKLDEDYEEEDEEEESLMWRAPKEEADYEDDFLEYDQEHIRFIDNMLMGSGAFVKKISLSPFSTTDSAYEWKMPKKSSLGSMPFSSDFEDFDYSSWDAMCYLDPSKAVEEDDFVVGFWNPSEENCGVDTGKQSISYDLHTEQCIADKSIADCVEALLGCYLTSCGERAAQLFLCSLGLKVLPVIKRTDREKALCPTRENFNSQQKNLSVSCAAASVASSRSSVLKDSEYGCLKIPPRCMFDHPDADKTLNHLISGFENFEKKINYRFKNKAYLLQAFTHASYHYNTITDCYQRLEFLGDAILDYLITKHLYEDPRQHSPGVLTDLRSALVNNTIFASLAVKYDYHKYFKAVSPELFHVIDDFVQFQLEKNEMQGMDSELRRSEEDEEKEEDIEVPKAMGDIFESLAGAIYMDSGMSLETVWQVYYPMMRPLIEKFSANVPRSPVRELLEMEPETAKFSPAERTYDGKVRVTVEVVGKGKFKGVGRSYRIAKSAAARRALRSLKANQPQVPNS

SEQ ID NO: 22 - DNA, 3912 bp, Artificial Sequence
  Artifically constructed sequence encoding amino acids 621-1922 of human DICER1 with 5′ ATG and miRNA target sequences mutated
  CDS (1)..(3912)
atgggcggacccagagtgaccatcaataccgccatcggccacatcaaccggtactgcgccagactgcccagcgacccttttacccacctggcccccaagtgccggaccagagaactgcctgacggcaccttctacagcaccctgtacctgcccatcaacagccctctgcgggcctctatcgtgggccctcctatgtcttgtgtgcggctggccgaaagagtggtggccctgatctgctgcgagaagctgcacaagatcggcgagctggacgaccacctgatgcccgtgggcaaagaaaccgtgaagtacgaggaagaactggacctgcacgatgaggaagagacaagcgtgcccggcagacccggcagcactaagagaaggcagtgctaccccaaggccatccccgagtgcctgagagacagctaccccagacccgaccagccttgctacctgtacgtgatcggcatggtgctgaccacccccctgcccgacgagctgaacttcagacggcggaagctgtacccccccgaggacaccaccagatgcttcggcatcctgaccgccaagcccatccctcagatcccccacttccccgtgtacaccagatccggcgaagtgacaatcagcatcgagctgaagaaaagcggcttcatgctgagcctgcagatgctggaactgatcacccggctgcaccagtacatcttcagccacatcctgcggctggaaaagcccgccctggaattcaagcccaccgacgccgatagcgcctactgcgtgctgcccctgaacgtcgtgaacgacagctccaccctggatatcgacttcaagttcatggaagatatcgagaagtccgaggcccggatcggcatccccagcaccaagtacaccaaagaaacacccttcgtgttcaagctggaagattaccaggacgccgtgatcatcccccggtacagaaacttcgatcagccccaccggttctacgtggccgacgtgtacacagacctgacccccctgtccaagttccctagcccagagtacgagacatttgccgagtactacaagacaaagtacaacctggatctgaccaacctgaaccagcctctgctggacgtggaccacaccagcagcagactgaacctgctgacccccagacacctgaatcagaagggcaaggccctgcccctgtcctctgccgagaagagaaaggccaagtgggagtctctgcagaacaagcagatcctggtgcccgagctgtgcgccatccaccctatccctgctagcctgtggcggaaggccgtgtgcctgcctagcatcctgtacagactgcactgcctgctgacagccgaggaactgagagcccagaccgcctctgatgctggcgtgggagtgcgctctctgcccgccgatttcagataccccaatctggacttcggctggaagaagtccatcgacagcaagagcttcatcagcatcagcaacagctccagcgccgaaaacgacaactactgcaagcacagcaccatcgtgcccgagaacgccgctcatcagggcgccaatagaaccagcagcctggaaaaccacgaccagatgagcgtgaactgccggaccctgctgagcgagtctcctggcaagctgcacgtggaagtgtccgccgacctgacagccatcaacggcctgagctacaaccagaacctggccaacggcagctacgatctggccaaccgggatttctgccagggcaaccagctgaattactacaagcaggaaatccccgtgcagcccaccaccagctacagcatccagaatctgtacagctacgagaaccagccacagccctccgacgagtgcaccctgctgtccaacaagtacctggatggcaacgccaacaagagcaccagcgacggctctcccgtgatggcagtgatgcctggcaccaccgacacaatccaggtgctgaagggccggatggacagcgagcagagccctagcatcggctacagcagcaggaccctgggccctaaccctggcctgattctgcaggccctgacactgagcaacgcctccgacggcttcaacctggaaagactggaaatgctgggcgacagcttcctgaagcacgccatcaccacctacctgttctgcacctaccccgacgcccacgagggcagactgtcctacatgcggagcaagaaggtgtccaactgcaacctgtaccggctgggcaagaagaagggcctgcccagcagaatggtggtgtctatcttcgacccccccgtgaactggctgccccctggctatgtcgtgaaccaggacaagagcaacaccgataagtgggagaaggacgagatgaccaaggactgcatgctggctaacggcaagctggatgaggactacgaagaagaagatgaggaagaagagtctctgatgtggcgggctcccaaagaggaagccgactacgaggacgacttcctggaatacgaccaggaacacatccggttcatcgacaacatgctgatgggctccggcgccttcgtgaagaagatcagcctgagcccctttagcaccacagacagcgcctacgagtggaagatgcccaagaagtcctccctgggcagcatgcctttcagcagcgacttcgaggacttcgactactccagctgggacgccatgtgctacctggatcccagcaaggccgtggaagaagatgacttcgtcgtgggcttctggaaccccagcgaagagaattgcggcgtggacaccggcaagcagagcatcagctacgacctgcacaccgagcagtgtatcgccgacaagtccattgccgactgcgtggaagccctgctgggctgctacctgacaagctgtggcgaaagagccgcccagctgttcctgtgtagcctgggcctgaaggtgctgcccgtgatcaagcggaccgaccgggaaaaggccctgtgccccaccagagagaacttcaacagccagcagaaaaacctgagcgtgtcctgcgccgctgccagcgtggcaagctctagaagcagcgtgctgaaagacagcgagtacggctgcctgaagatcccccccagatgcatgttcgaccaccccgatgccgacaagaccctgaaccatctgatcagcggcttcgagaatttcgagaagaaaatcaactaccggttcaagaacaaggcatacctgctgcaggccttcacacacgccagctaccactacaacaccatcaccgactgctaccagagactggaattcctgggagatgccatcctggattacctgatcaccaagcacctgtacgaggaccccagacagcacagccctggcgtgctgaccgatctgagaagcgccctcgtgaacaataccatcttcgccagcctggccgtgaaatacgactaccacaagtacttcaaagccgtgtccccagagctgttccacgtgatcgacgacttcgtgcagtttcagctggaaaaaaacgagatgcagggcatggactccgagctgcggagatccgaggaagatgaagagaaagaggaagatattgaggtgcccaaagccatgggcgacatctttgagagcctggctggcgccatctacatggatagcggcatgtctctggaaaccgtgtggcaggtgtactaccccatgatgcggcccctgatcgagaaattcagcgccaacgtgcccaggtcccccgtgcgggaactgctggaaatggaacccgagacagccaagttcagccctgccgagagaacctacgacggcaaagtgcgcgtgacagtggaagtcgtgggcaagggcaagttcaagggcgtgggcagaagctaccggatcgccaaatctgccgccgctagacgggctctgagatccctgaaggccaatcagcctcaggtgccaaacagctga

SEQ ID NO: 23 - protein, 1303 aa, Artificial Sequence; identical to SEQ ID NO: 21
  Synthetic Construct

(19) United States
(12) Patent Application Publication
(10) Pub. No.: US 2022/0401580 A1
(43) Pub. Date: Dec. 22, 2022

(54) OPTIDICER CONSTRUCT FOR AGE-RELATED MACULAR DEGENERATION

(71) Applicants: University of Virginia Patent Foundation, Charlottesville, VA (US); University of Utah Research Foundation, Salt Lake City, UT (US)

(72) Inventors: Jayakrishna Ambati, Charlottesville (US); Bradley David Unti Gelfand, Charlottesville, VA (US); Balamurali K. Ambati, Eugene (US); Hironori Uehara, Salt Lake City (US)

(73) Assignees: University of Virginia Patent Foundation, Charlottesville (US); University of Utah Research Foundation, Salt Lake City, UT (US)

(21) Appl. No.: 17/776,563

(22) PCT Filed: Nov. 12, 2020
(86) PCT No.: PCT/US2020/060232

§ 371 Date: May 12, 2022

Related U.S. Application Data
(60) Provisional application No. 62/934,168, filed on Nov. 12, 2019.

(51) Int. Cl. A61K 48/00; C12N 9/16; G01N 33/68; A61P 27/02

(52) U.S. Cl. CPC A61K 48/00; C12N 9/16; G01N 33/6893; A61P 27/02; C12N 2750/14143; G01N 2800/164; C12Y 301/26003; C07K 2319/00

(57) ABSTRACT

Provided are nucleotide sequences encoding polypeptides with ribonuclease III activity, wherein the nucleotide sequences have been modified to reduce their regulation by miRNAs. In some embodiments, the nucleotide sequences are at least 50% and as much as 100% identical to SEQ ID NO: 20 or SEQ ID NO: 22, and/or encode polypeptides that are at least 90% percent identical to SEQ ID NO: 23. Also provided are vectors and host cells that include the nucleotide sequences, methods for expressing the nucleotide sequences in cells, tissues, and organs, which in some embodiments can be in the eye of a subject in need thereof, methods for preventing and/or treating development of diseases or disorders and/or for restoring undesirably low DICER1 expression using the nucleotide sequences, and pharmaceutical compositions that have the presently disclosed nucleotide sequences.

### CROSS REFERENCE TO RELATED APPLICATION

This application is a United States National Stage application of PCT International Patent Application Serial No. PCT/US2020/060232, filed Nov. 12, 2020, which claims the benefit of U.S. Provisional Patent Application Ser. No. 62/934,168, filed Nov. 12, 2019, the disclosure of each of which incorporated herein by reference in its entirety.

### GRANT STATEMENT

This invention was made with government support under Grant Nos. EY017950, EY017182, EY028027, GM114862, EY022238, EY024068, EY026029, and EY029799 awarded by National Institutes of Health. The government has certain rights in the invention.

### TECHNICAL FIELD

The presently disclosed subject matter relates in some embodiments to compositions and methods for treating and/or preventing diseases and/or disorders of the eye in a mammal. More particularly, the presently disclosed subject matter relates to compositions encoding modified DICER1 polypeptides and methods for using the same to treat and/or prevent diseases and/or disorders of the eye in a mammal.

### BACKGROUND

Age-related macular degeneration (AMD) is a prevalent disease affecting an estimated one-in-forty people worldwide (Wong et al., 2014). In its advanced, blinding stages, AMD manifests as progressive atrophy of retinal pigmented epithelium (RPE), neuronal, and vascular components of the choroid and retina. In contrast to atrophic AMD, wet or neovascular AMD is typified by the invasion of immature blood vessels into the outer retina from the retina and choroid. Although characterized by apparently distinct pathological processes, atrophic and neovascular AMD are overlapping conditions, with both forms of AMD observed in fellow eyes of an individual (Sunness et al., 1999), within the same eye at sequential times, or even concurrently within the same eye (Kaszubski et al., 2016).

Deficiency of DICER1, an RNase that processes double-stranded and self-complementary RNAs including a majority of premature micro-RNAs (miRNAs) into their bioactive forms (Bernstein et al., 2001; Gan et al., 2006; Du et al., 2008), is among the inciting molecular events implicated in atrophic AMD (Kaneko et al., 2011; Dridi et al., 2012; Tarallo et al., 2012; Kim et al., 2014; Gelfand et al., 2015). DICER1 also metabolizes transcripts from short interspersed nuclear element (SINE) genetic repeats, principally Alu RNAs in humans and B1 and B2 RNAs in rodents (Murchison et al., 2007; Babiarz et al., 2008; Kaneko et al., 2011; Hu et al., 2012; Ohnishi et al., 2012; Ren et al., 2012; Flemr et al., 2013; Gelfand et al., 2015; Kim et al., 2016). DICER1 deficiency is implicated in RPE cell death in atrophic AMD due to accumulation of unprocessed Alu RNAs, which results in non-canonical activation of the NLRP3 inflammasome, an innate immune pathway resulting in caspase-1-dependent maturation of IL-1β and IL-18 and RPE death (Kaneko et al., 2011; Dridi et al., 2012; Tarallo et al., 2012; Kerur et al., 2013; Kim et al., 2014; Gelfand et al., 2015; Kerur et al., 2018).

Conversely, the extent to which DICER1 activity affects vascular homeostasis of the choroid and outer retina is largely unknown. The outer retina is normally avascular, situated between the retinal and choroidal vascular networks. Maintenance of these strict vascular boundaries is essential for vision; anatomic disruption and exudation from aberrant neovessels into the outer retinal space is responsible for blindness in a numerous ocular conditions including neovascular AMD, pathologic myopia, polypoidal choroidal vasculopathy, and angioid streaks.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

The presently disclosed subject matter provides in some embodiments nucleotide sequences encoding polypeptides with ribonuclease III activity. In some embodiments, the nucleotide sequence are at least 50%, 55%, 60%, 65%, 70%, 75%, 80%, 85%, 90%, 95%, 96%, 97%, 98%, or 99% percent identical to SEQ ID NO: 20 or SEQ ID NO: 22. In some embodiments, the polypeptides encoded by the nucleotide sequences are at least 90%, 95%, 96%, 97%, 98%, or 99% percent identical to SEQ ID NO: 23. In some embodiments, as compared to SEQ ID NO: 20, the nucleotide sequence comprises one or more nucleotide substitutions in one or more of the nucleotide position ranges of SEQ ID NO: 20 identified in Table 2, and further wherein the one or more nucleotide substitutions reduce or eliminate regulation of expression of an mRNA transcribed from SEQ ID NO: 20 by a member of an miRNA family listed in Table 2. In some embodiments, wherein as compared to SEQ ID NO: 20, the nucleotide sequence comprises one or more nucleotide substitutions in nucleotides 571-578, 778-784, 1784-1791, 1892-1899, and 3282-3289 of SEQ ID NO: 20, wherein the one or more nucleotide substitutions reduce or eliminate regulation of expression of an mRNA transcribed from SEQ ID NO: 20 by a member of the let-7 family of miRNAs. In some embodiments, the one or more nucleotide substitutions is/are silent with respect to the amino acid encoded by a codon comprising the one or more nucleotide substitutions as compared to the corresponding codon in SEQ ID NO: 20. In some embodiments, the nucleotide sequence comprises one or more nucleotide substitutions in one or more of the nucleotide position ranges of SEQ ID NO: 20 identified in Table 2, optionally in one or more of nucleotide position ranges 571-578, 778-784, 1784-1791, 1892-1899, and 3282-3289 of SEQ ID NO: 20, and further wherein the one or more nucleotide substitutions reduce or eliminate regulation of expression of an mRNA transcribed from SEQ ID NO: 20 by a member of an miRNA family listed in Table 2, optionally a member of the let-7 family of miRNAs, and further wherein the one or more nucleotide substitutions is/are silent with respect to the amino acid encoded by a codon comprising the one or more nucleotide substitutions as compared to the corresponding codon in SEQ ID NO: 20. In some embodiments, the nucleotide sequence comprises one or more nucleotide substitutions within each of nucleotide position ranges 571-578, 778-784, 1784-1791, 1892-1899, and 3282-3289 of SEQ ID NO: 20. In some embodiments, the nucleotide sequence encodes SEQ ID No: 23. In some embodiments, as compared to SEQ ID NO: 20, the nucleotide sequence comprises one or more nucleotide substitutions designed for codon optimization of the nucleotide sequence, optionally wherein the codon optimization is with respect to a expression of the nucleotide sequence in a human cell. In some embodiments, the nucleotide sequence encodes a polypeptide comprising, consisting essentially of, or consisting of an amino acid sequence at least 95% identical to SEQ ID NO: 23, wherein as compared to SEQ ID NO: 23, the nucleotide sequence encodes one or more conservative amino acid substitutions only. In some embodiments, the nucleotide sequence encodes a polypeptide comprising, consisting essentially of, or consisting of the amino acid sequence set forth in SEQ ID NO: 23.

The presently disclosed subject matter also relates in some embodiments to vectors, optionally expression vectors, comprising or consisting essentially of the nucleotide sequences disclosed herein. In some embodiments, the vector is an AAV vector.

The presently disclosed subject matter also relates in some embodiments to host cells comprising the presently disclosed nucleotide sequences and/or vectors.

The presently disclosed subject matter also relates in some embodiments to pharmaceutical compositions comprising the presently disclosed nucleotide sequences and/or vectors and a pharmaceutically acceptable diluent and/or excipient. In some embodiments, the pharmaceutically acceptable diluent and/or excipient is pharmaceutically acceptable for use in a human.

The presently disclosed subject matter also relates in some embodiments to methods for expressing a Δhel-DICER1 polypeptide in a cell. In some embodiments, the cell is a cell of the eye. In some embodiments, the cell is an RPE cell. In some embodiments, the methods comprise introducing into the cell a nucleotide sequence and/or a vector as disclosed herein or a pharmaceutical composition as disclosed herein.

The presently disclosed subject matter also relates in some embodiments to methods for preventing and/or treating development of diseases and/or disorders associated with undesirably low DICER1 expression, optionally undesirably low DICER1 expression in the eye, further optionally in the retina, and still further optionally in the RPE, of a subject in need thereof. In some embodiments, the methods comprise introducing into the eye, retina, and/or RPE a nucleotide sequence or vector as disclosed herein or a pharmaceutical composition as disclosed herein. In some embodiments, the disease or disorder is age-related macular degeneration (AMD). In some embodiments, the disease or disorder of the eye is associated with RPE degeneration, aberrant choroidal and retinal neovascularization (CRNV), or both.

The presently disclosed subject matter also relates in some embodiments to methods for restoring undesirably low DICER1 expression in the eye, optionally the retina, of a subject in need thereof. In some embodiments, the methods comprise administering to the subject a nucleotide sequence and/or a vector as disclosed herein and/or a pharmaceutical composition as disclosed herein.

In any of the methods disclosed herein, in some embodiments the nucleotide sequence and/or a vector as disclosed herein and/or a pharmaceutical composition as disclosed herein is administered by intravitreous injection; subretinal injection; episcleral injection; sub-Tenon's injection; retrobulbar injection; peribulbar injection; topical eye drop application; release from a sustained release implant device that is sutured to or attached to or placed on the sclera, or injected into the vitreous humor, or injected into the anterior chamber, or implanted in the lens bag or capsule; oral administration, intravenous administration; intramuscular injection; intraparenchymal injection; intracranial administration; intraarticular injection; retrograde ureteral infusion; intrauterine injection; intratesticular tubule injection; and any combination thereof.

The presently disclosed subject matter also relates in some embodiments to uses of the presently disclosed nucleotide sequences, vectors, and/or pharmaceutical compositions to express a Δhel-DICER1 polypeptide in a cell, optionally a cell of the eye, further optionally an RPE cell. In some embodiments, the cell is a human cell.

The presently disclosed subject matter also relates in some embodiments to uses of the presently disclosed nucleotide sequences, vectors, and/or pharmaceutical compositions to prevent and/or treat development of a disease or disorder of the eye, optionally the retina, further optionally the RPE, wherein the disease or disorder of the eye is associated with undesirably low DICER1 expression. In some embodiments, the disease or disorder is age-related macular degeneration (AMD). In some embodiments, the disease or disorder of the eye is associated with RPE degeneration, aberrant choroidal and retinal neovascularization (CRNV), or both.

The presently disclosed subject matter also relates in some embodiments to uses of the presently disclosed nucleotide sequences, vectors, and/or pharmaceutical compositions to restore undesirably low DICER1 expression in the eye, optionally the retina, of a subject.

The presently disclosed subject matter also relates in some embodiments to pharmaceutical compositions for preventing and/or treating diseases and/or disorders associated with undesirably low DICER1 expression, optionally in the eye, further optionally in the retina, and still further optionally in the RPE, of subject in need thereof. In some embodiments, the pharmaceutical compositions comprise an effective amount of the presently disclosed nucleotide sequences, vectors, and/or pharmaceutical compositions. In some embodiments, the disease and/or disorder of the eye is associated with RPE degeneration, aberrant choroidal and retinal neovascularization (CRNV), or both. In some embodiments, the effective amount restores undesirably low DICER1 expression in the eye, optionally the retina, of the subject. In some embodiments, the pharmaceutical compositions are formulated for administration by intravitreous injection; subretinal injection; episcleral injection; sub-Tenon's injection; retrobulbar injection; peribulbar injection; topical eye drop application; release from a sustained release implant device that is sutured to or attached to or placed on the sclera, or injected into the vitreous humor, or injected into the anterior chamber, or implanted in the lens bag or capsule; oral administration, intravenous administration; intramuscular injection; intraparenchymal injection; intracranial administration; intraarticular injection; retrograde ureteral infusion; intrauterine injection; intratesticular tubule injection; and any combination thereof.

In any of the embodiments of the presently disclosed subject matter, the disease, disorder, and/or condition associated with undesirably low DICER1 expression is selected from the group consisting of DICER1 syndrome, type 2 diabetes mellitus, diabetic retinopathy, age-related macular degeneration (AMD), RPE degeneration, aberrant choroidal and retinal neovascularization (CRNV), subretinal and retinal fibrosis, Fuchs' endothelial corneal dystrophy, Alzheimer's disease, rheumatoid arthritis, lupus, renal injury, tubulointerstitial fibrosis, glial axonal degeneration, idiopathic pulmonary fibrosis, lipid dysregulation, cholesterol accumulation associated with non-alcoholic steatohepatitis, clear cell renal cell carcinoma, atopic dermatitis, glomerulopathy, disorders of hypomyelination, tubal ectopic pregnancy and tubal abnormalities such as but not limited to cysts and disorganization of epithelial cells and smooth muscle cells, amyotrophic lateral sclerosis (ALS), Duchenne's muscular dystrophy, Sertoli cell deficiency/impaired spermatogenesis, and combinations thereof.

Accordingly, it is an object of the presently disclosed subject matter to provide compositions and methods for preventing and/or treating diseases and/or disorders of the eye associated with undesirably low DICER1 expression.

This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description, Figures, and EXAMPLES.

### BRIEF DESCRIPTIONS OF THE FIGURES

FIG. 1 depicts the results of sequencing crumbs family member 1, photoreceptor morphogenesis associated (Crb1) gene products isolated from C57BL/6J (wild type) and Dicer1^{d/d} mice confirming the absence of the rd8 mutation. The rd8 mutation results from a deletion of cytosine 3647 (asterisk) of the Mus musculus Crb1 mRNA as set forth in, for example, Accession No. NM_133239.2 of the GENBANK® biosequence database. The sequences shown (TTCTTATCGGTGTGCCT; SEQ ID NO: 13) are all identical to nucleotides 3640-3656 of Accession No. NM_133239.2 of the GENBANK® biosequence database.

FIG. 2 depicts quantitation of Dicer1 by quantitative real-time RT-PCR of cDNA (bar graph on left) and immunoblotting of protein (Western blot on right) from retina of littermate wild type and Dicer1^{d/d} mice.

FIG. 3A are representative fundus retinal photographs of age-matched 10-month-old wild type (WT) and Dicer1^{d/d} mice. Note focal hypopigmentation present in Dicer1^{d/d} eye denoted by red arrows. FIG. 3B show image-guided spectral-domain optical coherent tomography (SD-OCT) of a focal hypopigmented area of a Dicer1^{d/d} eye. Note outer retinal discontinuity denoted by red arrows. The left panel depicts the fundus image for image-guided SD-OCT. The yellow line denotes the location of the B-Scan OCT, depicted image in the right panel. FIG. 3C is a graph of the incidence of focal hypopigmentation, tabulated as percent of eyes, in wild type and littermate Dicer1^{d/d} with respect to age. n=48 Dicer1^{+/+} and 64 Dicer1^{d/d} examinations were included in this analysis. Age was significantly associated with incidence of hypopigmentation by linear regression p=0.0079. FIG. 3D depict toluidine blue-stained 1 μm thick sections of 15-month-old Dicer1^{d/d} (bottom) demonstrating vacuolar, atrophied RPE layer compared to wild type mice (top). FIG. 3E are two transmission electron micrographs of the basal aspect of RPE of 15-month-old wild type (top) and Dicer1^{d/d} (bottom). RPE from Dicer1^{d/d} mice exhibited large cytoplasmic vacuoles (V), loose basal infoldings (*) and debris at the interface of Bruch's membrane characteristic of basal laminar deposit (BLam). Scale bar=2 μm. FIG. 3F is a series of representative fluorescent micrographs of Dicer1^{d/d} and wild type littermate RPE flat mounts labeled with anti-Zonula Occludens-1 to label RPE tight junctions.

FIG. 4A is a series of fundus retinal images (left-most panel) and early, mid, and late fluorescein angiograms (second, third, and fourth panels, respectively) of wild type littermate and Dicer1^{d/d} mice. Black arrow in fundus retinal image denotes circular image artifact. Red arrow denotes focal hyperfluorescent neovascular lesion. FIG. 4B depicts an image guided SD-OCT of an angiographic lesion of a Dicer1^{d/d} mouse eye. FIG. 4C is a bar graph of the incidence and severity of neovascular lesions Dicer1^{d/d} with respect to age. Ninety individual examinations were included in this analysis. No vascular lesions were detected in wild type littermate mice at any age. Age was significantly associated with incidence and severity of neovascular lesions by linear regression (p=0.0184) and Spearman Rank (p<0.00058), respectively. FIG. 4D (top panel) is a hematoxylin and eosin-stained section showing a sub-RPE neovascular lesion retinal architecture in a 9-month-old Dicer1^{d/d} mouse. FIG. 4D (bottom panel) is a high magnification of a toluidine blue-stained 1 μm thick section of a neovascular lesion in a 12 month-old Dicer1^{d/d} mouse showing RPE delamination and migration. FIG. 4E is a series of representative early and late fluorescein angiograms of Dicer1^{d/d} mouse prior to and three days after intravitreous injection of Vegf neutralizing antibody or isotype. Red arrows denote neovascular lesion that resolved following Vegf neutralization.

FIG. 5 are two high-resolution micrographs of hematoxylin and eosin stained retina from Dicer1^{d/d} mice showing sub-RPE choroidal neovascularization (top panel) and chorioretinal anastomosis (bottom panel). Scale bar=50 μm.

FIG. 6 is a series of high-resolution bright field and fluorescent micrographs of choroidal vessels traversing Bruch's membrane (BM) in a Dicer1^{d/d} mouse. The top panel is a brightfield image. The middle panel is a fluorescent micrograph with VE-cadherin-positive labeling in yellow (white in black and white Figure) and nuclei labeled with DAPI in blue (gray in black and white Figure). The bottom panel is the overly of the top two panels. The white arrow denotes a VE-cadherin positive endothelial cell traversing Bruch's membrane.

FIG. 7 is a representative immunoblot (right panel) and densitometry quantification (bar graph on left) of Dicer1 abundance in retina from Dicer1^{H/H} relative to wild type littermate control mice.

FIG. 8A is a representative fundus retinal photograph of Dicer1^{H/H (}bottom panel) and littermate control (top panel). Black arrows denote camera artifact. Blue arrowheads (gray arrowheads in black and white Figure) denote patches of focal hypopigmentation. FIG. 8B is a series of representative early, middle, and late fluorescein angiograms showing active areas of neovascularization in Dicer1^{H/H} eyes (noted with arrows). No fluorescein leakage was detected in littermate wild type eyes. FIG. 8C is a representative image guided SD-OCT image of a neovascular lesion in a Dicer1^{H/H} mouse showing disruption of outer retinal architecture. The left panel depicts the fundus image for image-guided SD-OCT. The black horizontal line denotes the location of the B-Scan OCT image, depicted in the right panel. The black arrow in the fundus retinal image denotes circular image artifact. FIGS. 8D-8G are a series of hematoxylin and eosin-stained sections from wild type littermate and Dicer1^{H/H} eyes. Whereas wild type (FIG. 8D) and areas of Dicer1^{H/H} (FIG. 8E) appeared anatomically normal, focal areas of Dicer1^{H/H} mice exhibited RPE atrophy (FIG. 8F) and subretinal neovascular membranes (FIG. 8G).

FIG. 9 is a bar graph showing the results of quantitative RT-PCR of cDNA from whole retinas of 15-month old Dicer1^{d/d} (light gray bars) and littermate control (dark gray bars). n=3-4, *p<0.05.

FIG. 10 is a series of fluorescence micrographs of in situ fluorescent labeling caspase-1 activity in unfixed retinal cryo-sections of 10-month-old wild type and Dicer1^{d/d} mice. Green fluorescent signal indicated by arrows (lighter gray areas in black and white Figure) arose from a caspase-1 peptide substrate that became fluorescent upon cleavage. Signal was observed in the neovascular lesions.

FIG. 11A is a graph of analysis of the incidence focal hypopigmentation with respect to age in Dicer1^{d/d} (n=64 examinations), Dicer1^{d/d}; Casp1^{-/-}; Casp11^{-/-} (n=47), and Dicer1^{d/d}; Myd88^{-/-} (n=62). The effect of genotype on the presence of focal hypopigmentation was quantified by nominal regression using genotype and age as dependent variables and the presence or absence of focal hypopigmentation as an independent variable. Ablation of Casp1/Casp11 and Myd88 were associated with significantly reduced hypopigmentation ***p<0.001. FIGS. 11B and 11C show the results of angiogram grading of Dicer1^{d/d} (n=91), Dicer1^{d/d}; Casp1^{-/-}; Casp11^{-/-} (n=48), and Dicer1^{d/d}; Myd88^{-/-} (n=64). FIG. 11B is a graph of the incidence of vascular lesion positive eyes with respect to age. FIG. 11C is a bar graph showing the severity of neovascular lesions with respect to age. The effect of genotype on the severity of neovascular lesions was quantified by nominal regression using genotype and age as dependent variables and the neovascular lesion grade as an independent variable. Ablation of Casp1/Casp11 and Myd88 were associated with significantly reduced neovascular severity ***p<0.001.

FIGS. 12A and 12B are bar graphs of densitometry of Dicer1 abundance by immunoblotting of RPE (FIG. 12A) and retina (FIG. 12B) from wild type and JR5558 mice of indicated ages. n=5-11. Dicer1 levels were normalized to GAPDH. *p<0.05, **p<0.01 compared to wild type Dicer1 levels.

FIG. 13A depicts the results of in vitro processing assays of pre-miRNA of DICER1, Δhel-DICER1, and ΔPAZ-DICER1 purified from HEK 293T cells. FIG. 13B depict immunoblotting of HeLa and primary human RPE (hRPE) after transient transfection with plasmids to express GFP (pMaxGFP), Δhel-DICER1 (pΔhel-DICER1), or full-length human DICER1 (pDICER1). FIG. 13C depicts the time-course of Δhel-DICER1 expression in hRPE cells after transient transfection. Note faint detection of Δhel-DICER1 at 4 and 8 hours after transfection. FIG. 13D depicts the dose-dependent effect of dsRNA co-transfection on Δhel-DICER1 in hRPE. FIG. 13E shows the results of expression of endogenous and Δhel-DICER1 in primary hRPE 24 and 48 hours after transfection with indicated DICER1 constructs.

FIG. 14 is an immunoblot of purified DICER1 constructs expressed in HEK293T cells after transient transfection.

FIG. 15A is a blot showing detection of Δhel-DICER1 in retina following subretinal injection of AAV-OptiDicer. FIG. 15B is a series of representative fluorescein angiograms of JR5558 mice prior to, fourteen, and twenty-eight days after subretinal injection of AAV-OptiDicer or AAV-Empty. Injections were made in an area encompassing the lower left quadrant of the fundus relative to the optic nerve. Approximate injection site denoted by “*”. FIG. 15C is a series of bar graphs showing quantification of changes in total FA score and number of 2B lesions from baseline after AAV-OptiDicer- and AAV-Empty-injected eyes (n=7 eyes/treatment). *p<0.05, **p<0.01.

FIG. 16 is a sequence comparison between the nucleotide sequence of a Δhel-DICER1 construct (SEQ ID NO: 20; top strands, with the sequence denoted in lowercase) and the nucleotide sequence of an exemplary OptiDicer construct of the presently disclosed subject matter (SEQ ID NO: 22; bottom strands, with the sequence denoted in uppercase). The locations of various miRNA targets in the nucleotide sequence of the Δhel-DICER1 construct of SEQ ID NO: 20 are identified above each SEQ ID NO: sequence as a series of asterisks (see also Table 2). When target sequences carry across different lines in FIG. 16, this is indicated with “[+n], where n=the number of nucleotides in that target sequence that are continued on the next line. As can be see in FIG. 16, each and every miRNA target in SEQ ID NO: 20 has been modified by the introduction of at least one nucleotide change.

### BRIEF DESCRIPTION OF THE SEQUENCE LISTING

SEQ ID NOs: 1 and 2 are the nucleotide sequences of oligonucleotide primers that can be employed to analyze expression of the mouse Casp1 gene by real-time quantitative PCR.

SEQ ID NOs: 3 and 4 are the nucleotide sequences of oligonucleotide primers that can be employed to analyze expression of the mouse interleukin-1β gene by real-time quantitative PCR.

SEQ ID NOs: 5 and 6 are the nucleotide sequences of oligonucleotide primers that can be employed to analyze expression of the mouse interleukin-18 gene by real-time quantitative PCR.

SEQ ID NOs: 7 and 8 are the nucleotide sequences of oligonucleotide primers that can be employed to analyze expression of the mouse Nlrp3 gene by real-time quantitative PCR.

SEQ ID NOs: 9 and 10 are the nucleotide sequences of oligonucleotide primers that can be employed to analyze expression of the mouse 18S rRNA gene by real-time quantitative PCR.

SEQ ID NOs: 11 and 12 are the 5′ and 3′ nucleotide sequences, respectively, of an exemplary mouse Mirlet7a-1 microRNA.

SEQ ID NO: 13 is a subsequence of the mouse Crb1 coding sequence and corresponds to nucleotides 3640-3656 of Accession No. NM_133239.2 of the GENBANK® biosequence database.

SEQ ID NO: 14 is a nucleotide sequence corresponding to an exemplary full length human DICER1 gene product as disclosed in Accession No. NM_177438.2 of the GENBANK® biosequence database.

SEQ ID NO: 15 is an amino acid sequence encoded by the open reading frame (ORF) of SEQ ID NO: 14 and corresponds to Accession NO. NP_803187.1 of the GENBANK® biosequence database.

SEQ ID NO: 16 is a nucleotide sequence corresponding to an exemplary full length mouse Dicer1 gene product as disclosed in Accession No. NM_148948.2 of the GENBANK® biosequence database.

SEQ ID NO: 17 is an amino acid sequence encoded by the open reading frame (ORF) of SEQ ID NO: 16 and corresponds to Accession NO. NP_683750.2 of the GENBANK® biosequence database.

SEQ ID NOs: 18 and 19 are the nucleotide sequences of oligonucleotide primers that span exons 24 and 25 of the mouse Dicer1 cDNA and can be used to analyze expression of mouse Dicer1 by real-time quantitative PCR.

SEQ ID NO: 20 is the nucleotide sequence of a Δhel-DICER1 construct, which includes nucleotides 1906-5408 of the human DICER1 ORF (Accession No. NM_177438.2 of the GENBANK® biosequence database) with an initiator methionine codon added to the 5′ end. There is also an A to C change at nucleotide 225 of SEQ ID NO: 20, which corresponds to nucleotide 2320 of Accession No. NM_177438.2 of the GENBANK® biosequence database.

SEQ ID NO: 21 is the amino acid sequence encoded by SEQ ID NO: 21. Amino acids 2-1303 are 100% identical to amino acids 621-1922 of Accession NO. NP_803187.1 of the GENBANK® biosequence database.

SEQ ID NO: 22 is the nucleotide sequence of an exemplary OptiDicer construct of the presently disclosed subject matter. It is 75% identical to SEQ ID NO: 20, with the differences constituting nucleotide substitutions designed to destroy target sequences for various regulatory miRNAs that are found in the DICER1 coding sequences included within the Δhel-DICER1 construct and additional nucleotide changes designed for codon optimization.

SEQ ID NO: 23 is the amino acid sequence encoded by SEQ ID NO: 22. It is 100% identical to amino acids 621-1922 of Accession NO. NP_803187.1 of the GENBANK® biosequence database, and includes an N-terminal methionine.

### DETAILED DESCRIPTION

Disclosed herein are analyses using two exemplary mouse models of DICER1 deficiency, analysis of a third spontaneous model of choroidal neovascularization, and restorative gene transfer which collectively reveal that, in addition to promoting RPE atrophy, chronic DICER1 deficiency also stimulates pathological choroidal and retinal neovascularization. These findings significantly expand the repertoire of DICER1 activities in maintaining choroidal and retinal vascular homeostasis in pathological processes that impair the vision of millions of individuals.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some embodiments all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a”, “an”, and “the” refer to “one or more” when used in this application, including in the claims. For example, the phrase “an antibody” refers to one or more antibodies, including a plurality of the same antibody. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The terms “additional therapeutically active compound” and “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease, or disorder being treated.

As used herein, the term “adjuvant” refers to a substance that elicits an enhanced immune response when used in combination with a specific antigen.

As use herein, the terms “administration of” and/or “administering” a compound should be understood to refer to providing a compound of the presently disclosed subject matter to a subject in need of treatment.

With regard to administering a composition, the term “administering” as used herein refers to any method for providing a composition and/or pharmaceutical composition thereof to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, subcutaneous administration, intravitreous administration, including via intravitreous sustained drug delivery device, intracameral (into anterior chamber) administration, suprachoroidal injection, subretinal administration, subconjunctival injection, sub-Tenon's administration, peribulbar administration, transscleral drug delivery, intravenous injection, intraparenchymal/intracranial injection, intra-articular injection, retrograde ureteral infusion, intrauterine injection, intratesticular tubule injection, intrathecal injection, intraventricular (e.g., inside cerebral ventricles) administration, administration via topical eye drops, and the like. Administration can be continuous or intermittent. In some embodiments, a preparation can be administered therapeutically; that is, administered to treat an existing disease or condition. In some embodiments, a preparation can be administered prophylactically; that is, administered for prevention of a disease, disorder, or condition.

As used herein, “amino acids” are represented by the full name thereof, by the three letter code corresponding thereto, or by the one-letter code corresponding thereto, as indicated in Table 1:

TABLE 1
Amino Acids, Abbreviations Thereof,
and Functionally Equivalent Codons

| | 3-Letter | 1-Letter | Functionally |
|---|---|---|---|
| Full Name | Code | Code | Equivalent Codons |
| Aspartic Acid | Asp | D | GAC; GAU |
| Glutamic Acid | Glu | E | GAA; GAG |
| Lysine | Lys | K | AAA; AAG |
| Arginine | Arg | R | AGA; AGG; CGA; CGC; CGG; |
| | | | CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA; CAG |
| Serine | Ser | S | ACG; AGU; UCA; UCC; UCG; |
| | | | UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; CUC; CUG; |
| | | | CUU |
| Isoleucine | Ile | I | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation, and/or substitution with other chemical groups which can change the peptides' circulating half-lives without adversely affecting their activities. Additionally, a disulfide linkage may be present or absent in the peptides of the presently disclosed subject matter.

The term “amino acid” is used interchangeably with “amino acid residue”, and may refer to a free amino acid and/or to an amino acid residue of a peptide. It will be apparent from the context in which the term is used whether it refers to a free amino acid or a residue of a peptide.

Amino acids have the following general structure:

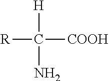

Amino acids may be classified into seven groups on the basis of the side chain R: (1) aliphatic side chains; (2) side chains containing a hydroxylic (OH) group; (3) side chains containing sulfur atoms; (4) side chains containing an acidic or amide group; (5) side chains containing a basic group; (6) side chains containing an aromatic ring; and (7) proline, an imino acid in which the side chain is fused to the amino group.

Synthetic or non-naturally occurring amino acids refer to amino acids which do not naturally occur in vivo but which, nevertheless, can be incorporated into the peptide structures described herein. The resulting “synthetic peptide” contain amino acids other than the 20 naturally occurring, genetically encoded amino acids at one, two, or more positions of the peptides. For instance, naphthylalanine can be substituted for tryptophan to facilitate synthesis. Other synthetic amino acids that can be substituted into peptides include L-hydroxypropyl, L-3,4-dihydroxyphenylalanyl, alpha-amino acids such as L-α-hydroxylysyl and D-α-methylalanyl, L-α-methylalanyl, β-amino acids, and isoquinolyl. D-amino acids and/or non-naturally occurring synthetic amino acids can also be incorporated into the peptides of the presently disclosed subject matter. Other derivatives include replacement of the naturally occurring side chains of the 20 genetically encoded amino acids (or any L- or D-amino acid) with other side chains.

As used herein, the term “silent mutation” refers to one or more nucleotide changes that in the context of a coding sequence do not result in an amino acid change in the polypeptide encoded by the coding sequence. One of ordinary skill in the art can determine silent mutations for most although not all of the naturally occurring amino acids by reference to the genetic code summarized in the Table above, particularly the functionally equivalent codons. Silent mutations can involve single nucleotide changes (e.g., GAC to GAU or vice versa for aspartic acid; a change of one of CGA, CGC, CGG, or CGU to one of the other three for arginine; a change of one of ACA, ACC, ACG, or ACU to one of the other three for threonine; etc.). However, silent mutations need not be single nucleotide changes. By way of example and not limitation, the codons ACG, AGU, UCA, UCC, UCG, and UCU all code for serine, so a change from ACG to AGU or even from to AGU to UCG would constitute a silent mutation as that term is used herein.

As used herein, the term “conservative amino acid substitution” is defined herein as exchanges within one of the following five groups:

I. Small aliphatic, nonpolar or slightly polar residues: Ala, Ser, Thr, Pro, Gly;

II. Polar, negatively charged residues and their amides: Asp, Asn, Glu, Gln;

III. Polar, positively charged residues: His, Arg, Lys;

IV. Large, aliphatic, nonpolar residues: Met Leu, Ile, Val, Cys

V. Large, aromatic residues: Phe, Tyr, Trp

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample”, as used herein, refers to samples obtained from a subject, including but not limited to skin, hair, tissue, blood, plasma, cells, sweat, and urine.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids (e.g., two DNA molecules). When a nucleotide position in both of the molecules is occupied by nucleotides normally capable of base pairing with each other at a given position, the nucleic acids are considered to be complementary to each other at this position. Thus, two nucleic acids are complementary to each other when a substantial number (in some embodiments at least 50%) of corresponding positions in each of the molecules are occupied by nucleotides that can base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. By way of example and not limitation, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

A “compound”, as used herein, refers to a polypeptide, an isolated nucleic acid, or other agent used in the method of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a condition, disease, or disorder for which the test is being performed.

A “test” cell is a cell being examined.

A “pathoindicative” cell is a cell that, when present in a tissue, is an indication that the animal in which the tissue is located (or from which the tissue was obtained) is afflicted with a condition, disease, or disorder.

A “pathogenic” cell is a cell that, when present in a tissue, causes or contributes to a condition, disease, or disorder in the animal in which the tissue is located (or from which the tissue was obtained).

A tissue “normally comprises” a cell if one or more of the cell are present in the tissue in an animal not afflicted with a condition, disease, or disorder.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition, and are encompassed within the nature of the phrase “consisting essentially of”.

With respect to the terms “comprising”, “consisting of”, and “consisting essentially of”, where one of these three terms is used herein, the presently disclosed and claimed subject matter can include the use of either of the other two terms. For example, in some embodiments, the presently disclosed subject matter relates to compositions comprising antibodies. It would be understood by one of ordinary skill in the art after review of the instant disclosure that the presently disclosed subject matter thus encompasses compositions that consist essentially of the antibodies of the presently disclosed subject matter, as well as compositions that consist of the antibodies of the presently disclosed subject matter.

As used herein, the terms “condition”, “disease condition”, “disease”, “disease state”, and “disorder” refer to physiological states in which diseased cells or cells of interest can be targeted with the compositions of the presently disclosed subject matter. Any cell for which expression of a Dicer1 gene product might be desirable can be targeted with the compositions of the presently disclosed subject matter, and any disease, disorder, or condition associated with undesirably low expression of Dicer1 can be treated, and/or a symptom thereof can be ameliorated, using the compositions of the presently disclosed subject matter. Similarly, any disease, disorder, or condition associated with undesirably low expression of Dicer1 can be prevented and/or delayed in its development using the compositions of the presently disclosed subject matter. A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the phrase “associated with associated with undesirably low DICER1 expression” refers to any disease, disorder, or condition, or a symptom thereof, which results either directly or indirectly from expression of a DICER1 gene product in a cell, tissue, or organ that is below that necessary to prevent the disease, disorder, or condition, or the symptom thereof, from occurring. Stated another way, the phrase relates to diseases, disorders, conditions, and symptoms thereof that result from DICER1 expression that is lower than would have been present in the cell, tissue, or organ of a subject with a normal level of DICER1 expression in that same cell, tissue, or organ. Various diseases, disorders, conditions, and symptoms thereof are associated with associated with undesirably low DICER1 expression, which include but are not limited to aberrant choroidal and retinal neovascularization (CRNV), acne vulgaris, acute and chronic bone marrow transplant rejection, acute and chronic organ transplant rejection, acute tubular injury, age-related macular degeneration (AMD), allergic asthma, Alzheimer's disease, amyotrophic lateral sclerosis (ALS), anxiety disorders, atherosclerosis, atopic dermatitis, autoimmune hepatitis, polycystic kidney disease including but not limited to autosomal dominant polycystic kidney disease, bipolar disorder, breast cancer, Burkholderia cenocepacia infection, cardiac surgery (peri-/post-operative inflammation), Chlamydia spp., cholesterol accumulation associated with non-alcoholic steatohepatitis, chronic infantile neurologic cutaneous and articular autoinflammatory diseases, chronic inflammatory and neuropathic pain, chronic lymphocytic leukemia, chronic obstructive pulmonary disease, chronic pain, clear cell renal cell carcinoma, colon cancer, contact dermatitis, Crohn's disease, Cryopyrinopathies, cystic fibrosis, diabetic nephropathy, disorders of hypomyelination, drug-induced lung inflammation, Duchenne's muscular dystrophy, familial cold autoinflammatory syndrome, Francisella spp., Fuchs' endothelial corneal dystrophy, glaucoma, glial axonal degeneration, glomerulonephritis, glomerulopathy, gout, graft vascular injury, graft-versus-host disease, gram negative sepsis, hay fever, helminth parasites, hemolytic-uremic syndrome, high-grade urothelial carcinoma, Huntington's disease, hypertension, idiopathic pulmonary fibrosis, IgA nephropathy, immune complex renal disease, infectious Pseudomonas aeruginosa, inflammatory joint disease, insulin resistance, irritable bowel syndrome, ischemic heart disease, ischemic stroke, keratitis, kidney clear cell carcinoma, Legionella spp., Leishmania spp. leprosy, lipid dysregulation, lupus nephritis, lupus, major depressive disorder, malaria, melanoma, metabolic syndrome, Muckle-Wells syndrome and neonatal onset multisystem inflammatory disease, mucoid colon cancer, multiple sclerosis, nephritis, neuroblastoma, neuroendocrine cancer, neuropathic pain, non-alcoholic fatty liver disease, obesity, osteoporosis in post-menopausal women and fracture patients, osteoporosis, papillary intracystic breast carcinoma, Parkinson's disease, polyoma virus infection, proliferative vitreoretinopathy, prostate cancer, psoriasis, pulmonary fibrosis, pulmonary tuberculosis, reactive arthritis, renal fibrosis, renal injury, renal ischemia-perfusion injury, respiratory syncitial virus infection, rheumatoid arthritis, RPE degeneration, type 2 diabetes mellitus, diabetic retinopathy, DICER1 syndrome (see e.g., Robertson et al., 2018), salivary gland inflammation, scleroderma, septic shock, Sertoli cell deficiency/impaired spermatogenesis, Sjogren's syndrome, skin cancer, spinal cord injury, subretinal and retinal fibrosis, syphilis, systemic lupus erythematosus, systemic vasculitides, thrombosis, thyroid cancer, traumatic brain injury, tubal ectopic pregnancy and tubal abnormalities such as but not limited to cysts and disorganization of epithelial cells and smooth muscle cells, tubular early gastric cancer, tubulointerstitial fibrosis, tumor angiogenesis, type I diabetes, type II diabetes, ulcerative colitis, undifferentiated ovarian carcinoma, varicose veins, Vibrio cholera, yoglobulinemia, and any combination thereof.

As used herein, the term “diagnosis” refers to detecting a risk or propensity to a condition, disease, or disorder. In any method of diagnosis exist false positives and false negatives. Any one method of diagnosis does not provide 100% accuracy.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene encodes a protein if transcription and translation of an mRNA corresponding to or derived from that gene produces the protein in a cell or other biological system and/or an in vitro or ex vivo system. Both the coding strand, the nucleotide sequence of which is identical to the mRNA sequence (with the exception of uracil bases presented in the latter) and is usually provided in Sequence Listing, and the non-coding strand, used as the template for transcription of a gene or cDNA, can be referred to as encoding the protein or other product of that gene or cDNA.

As used herein, an “essentially pure” preparation of a particular protein or peptide is a preparation wherein in some embodiments at least about 95% and in some embodiments at least about 99%, by weight, of the protein or peptide in the preparation is the particular protein or peptide.

A “fragment”, “segment”, or “subsequence” is a portion of an amino acid sequence, comprising at least one amino acid, or a portion of a nucleic acid sequence comprising at least one nucleotide. The terms “fragment”, “segment”, and “subsequence” are used interchangeably herein.

As used herein, a “functional” biological molecule is a biological molecule in a form in which it exhibits a property by which it can be characterized. A functional enzyme, for example, is one that exhibits the characteristic catalytic activity by which the enzyme can be characterized.

As used herein, the term “homologous” refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein “injecting”, “applying”, and administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids that have been substantially purified from other components which naturally accompany the nucleic acid it in a cell, e.g., RNA or DNA or proteins. The term therefore includes, for example, a recombinant DNA which is incorporated into a vector, an autonomously replicating plasmid or virus, or the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to through ionic or hydrogen bonds or van der Waals interactions.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the presently disclosed subject matter.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

As used herein, the term “nucleic acid” also encompasses RNA as well as single and double-stranded DNA and cDNA. Furthermore, the terms “nucleic acid”, “DNA”, “RNA”, and similar terms also include nucleic acid analogs, e.g., the so-called “peptide nucleic acids”, which are known in the art and have peptide bonds instead of phosphodiester bonds in the backbone, are considered within the scope of the presently disclosed subject matter.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, which in some embodiments are no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

By describing two or more polynucleotides as “operably linked” it is meant that a single-stranded or double-stranded nucleic acid comprises the two or more polynucleotides arranged within a nucleic acid molecule in such a manner that at least one of the two or more polynucleotides is able to exert a physiological effect by which it is characterized upon the other. By way of example, a promoter operably linked to the coding region of a gene is able to promote transcription of the coding region.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

A “preventive” or “prophylactic” treatment is a treatment administered to a subject who does not exhibit signs, or exhibits only early signs, of a condition, disease, or disorder. A prophylactic or preventative treatment is administered for the purpose of decreasing the risk of developing pathology associated with developing the condition, disease, or disorder.

“Primer” refers to a polynucleotide that is capable of specifically hybridizing to a designated polynucleotide template and providing a point of initiation for synthesis of a complementary polynucleotide (e.g., polymerization). Such synthesis occurs when the polynucleotide primer is placed under conditions in which synthesis is induced, e.g., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. In some embodiments, primers can be labeled, e.g., with chromogenic, radioactive, and/or fluorescent moieties and used as detectable moieties.

As used herein, the term “promoter/regulatory sequence” means a nucleic acid sequence which is required for expression of a gene product operably linked to the promoter/regulator sequence. In some embodiments, this sequence may be the core promoter sequence, and in some embodiments, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a cell substantially only if the cell is a cell of the tissue type corresponding to the promoter.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is in some embodiments greater than 90% pure, that is in some embodiments greater than 95% pure, and that is in some embodiments greater than 98% pure.

“Recombinant polynucleotide” refers to a polynucleotide having sequences that are not generally found joined together in nature. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide produces a “recombinant polypeptide”.

A “recombinant polypeptide” is one which is produced upon expression of a recombinant polynucleotide, in some embodiments by a recombinant host cell.

As used herein, the term “mammal” refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

As used herein, the phrase “Dicer1” refers to genes and gene products identified as dicer 1, ribonuclease type III. In humans, the DICER1 locus is present on chromosome 14 and corresponds to the reverse-complement of nucleotides 95,086,228-95,157,422 of Accession No. NC_000014.9 of the GENBANK® biosequence database. An exemplary human cDNA is disclosed as Accession No. NM_177438.2 of the GENBANK® biosequence database, which encodes a polypeptide with the amino acid sequence disclosed as Accession No. NP_803187.1 of the GENBANK® biosequence database. In Mus musculus, the Dicer1 locus is present on chromosome 12 and corresponds to the reverse-complement of nucleotides 104,687,742-104,751,952 of Accession No. NC_000078.6 of the GENBANK® biosequence database. An exemplary Mus musculus cDNA is disclosed as Accession No. NM_148948.2 of the GENBANK® biosequence database, which encodes a polypeptide with the amino acid sequence disclosed as Accession No. NP_683750.2 of the GENBANK® biosequence database.

The term “polynucleotide” as used herein includes but is not limited to DNA, RNA, complementary DNA (cDNA), messenger RNA (mRNA), ribosomal RNA (rRNA), small hairpin RNA (shRNA), small nuclear RNA (snRNA), short nucleolar RNA (snoRNA), microRNA (miRNA), genomic DNA, synthetic DNA, synthetic RNA, and/or tRNA.

The term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichythyes (bony fish), Amphibia (amphibians), Reptilia (reptiles), Ayes (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, in some embodiments, humans.

As used herein, a “subject in need thereof” is a patient, animal, mammal, or human, who will benefit from the method of this presently disclosed subject matter.

As used herein, “substantially homologous amino acid sequences” includes those amino acid sequences which have in some embodiments at least about 95% homology, in some embodiments at least about 96% homology, in some embodiments at least about 97% homology, in some embodiments at least about 98% homology, and in some embodiments at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is in some embodiments at least about 50%, 65%, 75%, 85%, 95%, 99%, or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: in some embodiments in 7% sodium dodecyl sulfate SDS, 0.5 M NaPO₄, 1 mM EDTA at 50° C. with washing in 2× standard saline citrate (SSC), 0.1% SDS at 50° C.; in some embodiments in 7% (SDS), 0.5 M NaPO₄, 1 mM EDTA at 50° C. with washing in 1× SSC, 0.1% SDS at 50° C.; in some embodiments in 7% SDS, 0.5 M NaPO₄, 1 mM EDTA at 50° C. with washing in 0.5×SSC, 0.1% SDS at 50° C.; and in some embodiments in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990a,b; Altschul et al., 1997). The default settings provided with these programs are suitable for determining substantial similarity of nucleic acid sequences for purposes of the presently disclosed subject matter.

The term “substantially pure” describes a compound, e.g., a protein or polypeptide, which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

As used herein, the term “transduction” refers to the introduction of a foreign nucleic acid into a cell using a vector, in some embodiments a viral vector.

As used herein, the term “transfection” as used herein refers to the introduction of a foreign nucleic acid into a cell using recombinant DNA technology. The term “transformation” means the introduction of a “foreign” (i.e., extrinsic or exogenous) gene, DNA, or RNA sequence to a host cell, such that the host cell will express the introduced gene or sequence to produce a desired substance, such as a protein or enzyme, coded by the introduced gene or sequence. The introduced gene or sequence can also be called a “cloned”, “foreign”, or “heterologous” gene or sequence or a “transgene”, and can include regulatory and/or control sequences, such as start, stop, promoter, signal, secretion, or other sequences used by a cell's genetic machinery. The gene or sequence can include nonfunctional sequences or sequences with no known function. A host cell that receives and expresses introduced DNA or RNA has been “transformed” and is a “transformant” or a “clone”, and is “transgenic”. The DNA or RNA introduced to a host cell can come from any source, including cells of the same genus or species as the host cell, or cells of a different genus or species

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

As used herein, the terms “vector”, “cloning vector”, and “expression vector” refer to a vehicle by which a polynucleotide sequence (e.g., a foreign gene) can be introduced into a host cell, so as to transduce and/or transform the host cell in order to promote expression (e.g., transcription and translation) of the introduced sequence. Vectors include plasmids, phages, viruses, etc.

As used herein, the term “expression vector” refers to a DNA sequence capable of directing expression of a particular nucleotide sequence in an appropriate host cell, comprising a promoter operatively linked to the nucleotide sequence of interest which is operatively linked to termination signals. It also typically comprises sequences required for proper translation of the nucleotide sequence. The construct comprising the nucleotide sequence of interest can be chimeric. The construct can also be one that is naturally occurring but has been obtained in a recombinant form useful for heterologous expression. In some embodiments, the expression vector comprises an nucleic acid molecule of the presently disclosed subject matter, which in some embodiments comprises, consists essentially of, or consists of SEQ ID NO: 20 or 22, or a variant or derivative thereof, and/or that encodes SEQ ID NO: 21 or 23, or a variant or derivative thereof.

Similarly, all genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the human DICER1 gene products presented in Accession Nos: NM_177438.2 (SEQ ID NO: 14) and NP_803187.1 (SEQ ID NO: 15) of the GENBANK® biosequence database are intended to encompass homologous genes and gene products from other animals including, but not limited to other mammals, fish, amphibians, reptiles, and birds, including the murine Dicer1 gene products presented in Accession Nos: NM_148948.2 (SEQ ID NO: 16) and NP_683750.2 (SEQ ID NO: 17) of the GENBANK® biosequence database. Also encompassed are any and all nucleotide and amino acid sequences that correspond to and/or are encoded by transcript variants of these sequences, including but not limited to those disclosed in Accession Nos. NM_030621.4, NM_001195573.1, NM_001271282.3, and NM_001291628.1 of the GENBANK® biosequence database, which encode the amino acid sequences disclosed in Accession Nos. NP_085124.2, NP_001182502.1, NP_001258211.1, and NP_001278557.1 of the GENBANK® biosequence database, respectively.

As used herein, the terms “operatively linked” and “operably linked refer to transcriptional regulatory elements (such as, but not limited to promoter sequences, transcription terminator sequences, etc.) that are connected to a nucleotide sequence (for example, a coding sequence or open reading frame) in such a way that the transcription of the nucleotide sequence is controlled and regulated by that transcriptional regulatory element. Similarly, a nucleotide sequence is said to be under the “transcriptional control” of a promoter to which it is operably linked. Techniques for operatively linking a promoter region to a nucleotide sequence are known in the art.

### II. Compositions

II.A. Nucleotides Sequences, Vectors, and Host Cells Comprising the Same

In some embodiments, the presently disclosed subject matter provides nucleotide sequences that encode polypeptides with ribonuclease III activity, more particularly with DICER1 activity. Exemplary human DICER1 gene products are presented in Accession Nos: NM_177438.2 (SEQ ID NO: 14) and NP_803187.1 (SEQ ID NO: 15) of the GENBANK® biosequence database, and exemplary murine Dicer1 gene products presented in Accession Nos: NM_148948.2 (SEQ ID NO: 16) and NP_683750.2 (SEQ ID NO: 17) of the GENBANK® biosequence database, and in some embodiments the nucleotide sequences of the presently disclosed subject matter are variants and/or derivatives of the human DICER1 and/or murine Dicer1 gene sequences.

In some embodiments, the nucleotide sequences of the presently disclosed subject matter include modifications of the human and/or murine Dicer1 sequences to remove sequences that encode the N-terminal helicase domain of Dicer1. In some embodiments, the nucleotide sequences of the presently disclosed subject matter thus include and/or are based on a deletion of nucleotides 1-2098 of Accession No: NM_177438.2 (SEQ ID NO: 14) of the GENBANK® biosequence database, which include the N-terminal helicase domain coding sequences of DICER1. Therefore, in some embodiments the nucleotide sequences of the presently disclosed subject matter include and/or are based on nucleotides 2099-6077 of Accession No: NM_177438.2 (SEQ ID NO: 14) of the GENBANK® biosequence database, optionally with an initiator methionine at the 5′ end. An exemplary such nucleotide sequence is presented in SEQ ID NO: 20 and referred to herein as Δhel-DICER1. SEQ ID NO: 20 encodes the polypeptide of SEQ ID NO: 23.

As disclosed herein, in some embodiments the nucleotide sequences of the presently disclosed subject matter are designed to encode a polypeptide of SEQ ID NO: 23, but include one or more modifications of the nucleotide sequence of SEQ ID NO: 20 such that codon usage is optimized.

Alternatively or in addition, relative to SEQ ID NO: 20 the nucleotide sequences of the presently disclosed subject can also be modified to disrupt regulation of the biological activities of the gene products to which the nucleotide sequences correspond by miRNAs. It is known that miRNAs function as gene expression regulators, and as disclosed herein, Dicer1 is a gene for which miRNA-mediated regulation has been established. To this end, in some embodiments the nucleotide sequences of the presently disclosed subject matter include particular nucleotide substitutions that relative to SEQ ID NO: 20 are designed to interfere with binding of one or more miRNAs to Dicer1 transcription products. These nucleotide substitutions can in some embodiments also be silent mutations such that the nucleotide sequences of the presently disclosed subject matter encode polypeptides that have the same amino acid sequence as, for example, the human DICER1 gene product presented in Accession No: NP_803187.1 (SEQ ID NO: 15) of the GENBANK® biosequence database.

Therefore, in some embodiments and as compared to SEQ ID NO: 20, the nucleotide sequences of the presently disclosed subject matter comprise one or more nucleotide substitutions in one or more of the nucleotide position ranges of SEQ ID NO: 20 identified in Table 2, which in some embodiments reduce or eliminate regulation of expression of an mRNA transcribed from SEQ ID NO: 20 by a member of an miRNA family listed in Table 2.

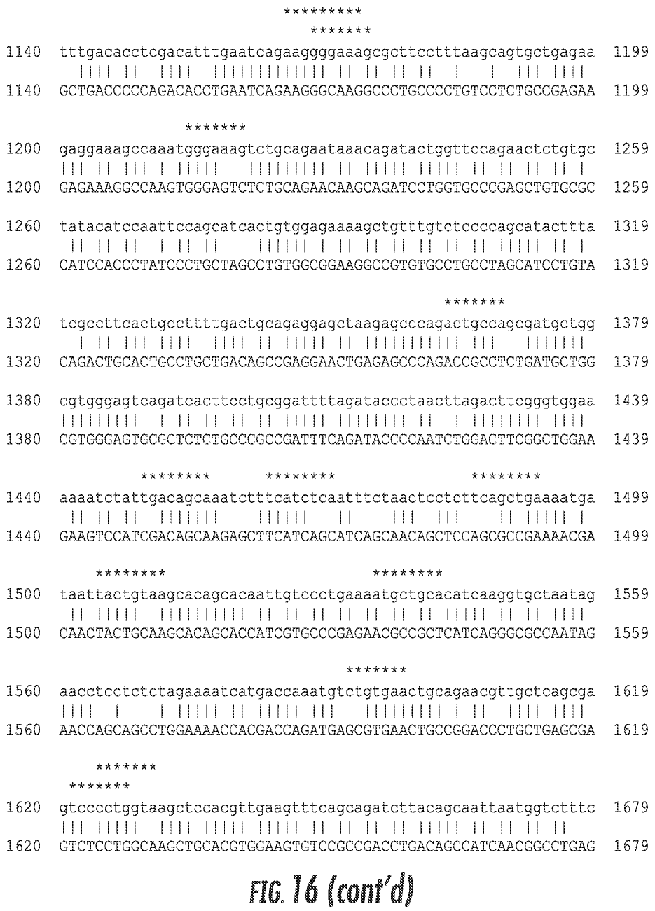

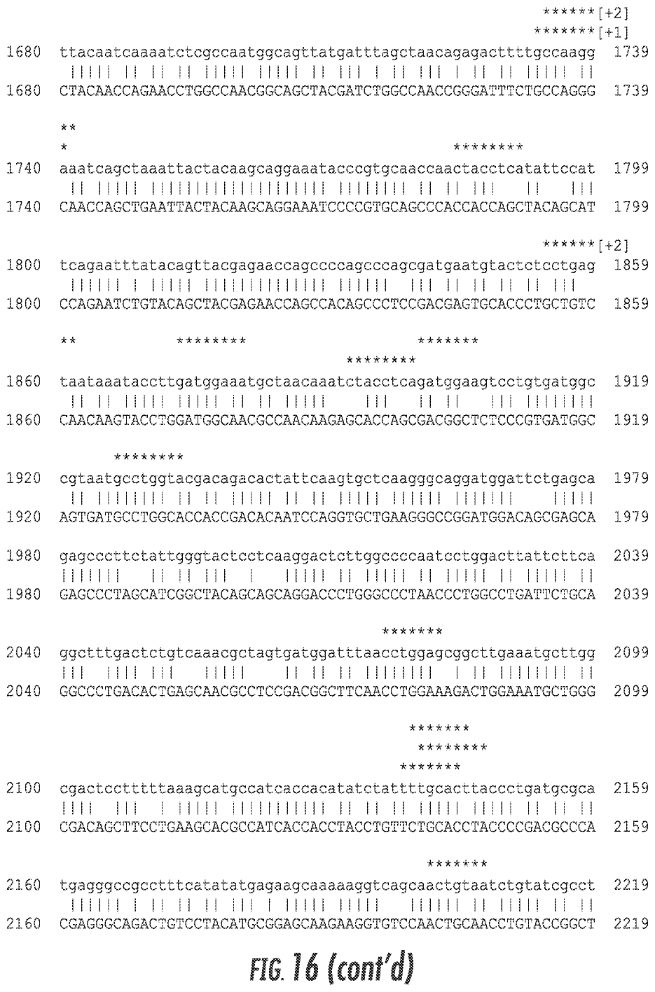

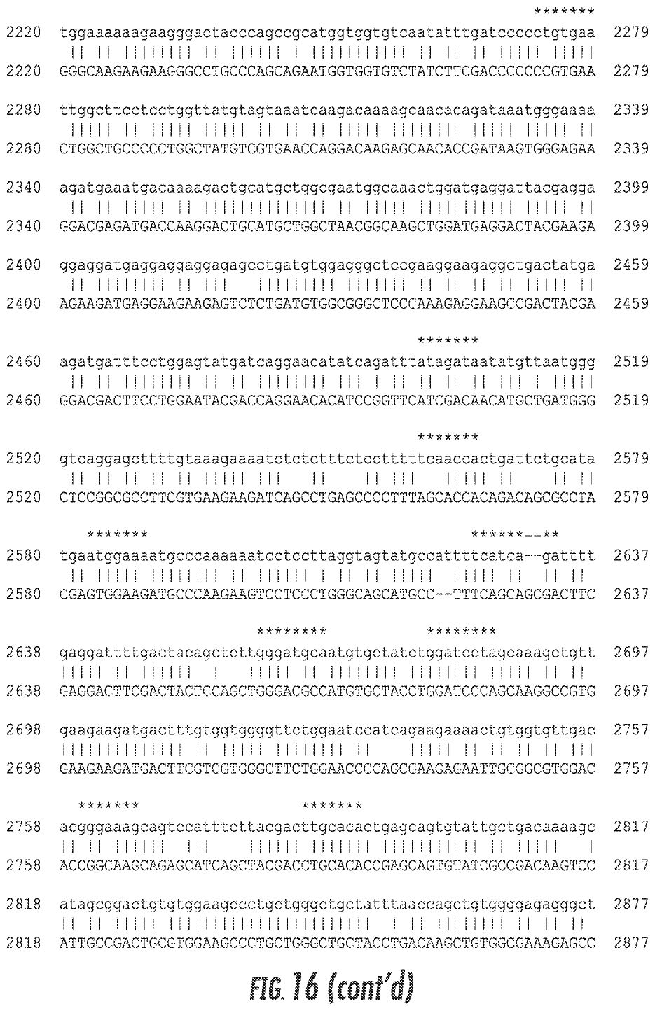

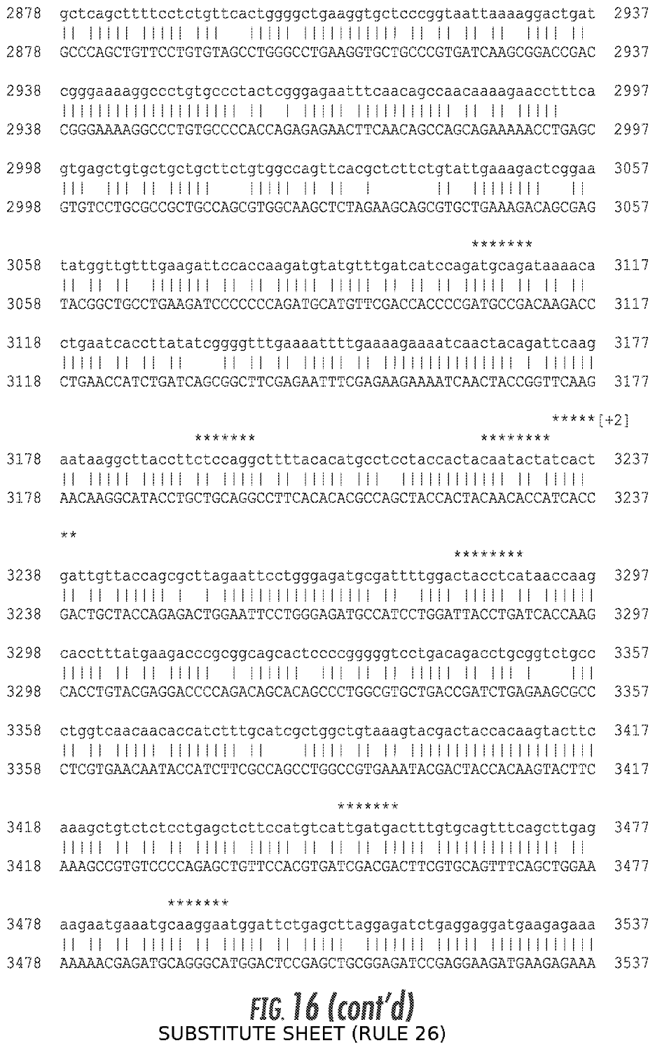

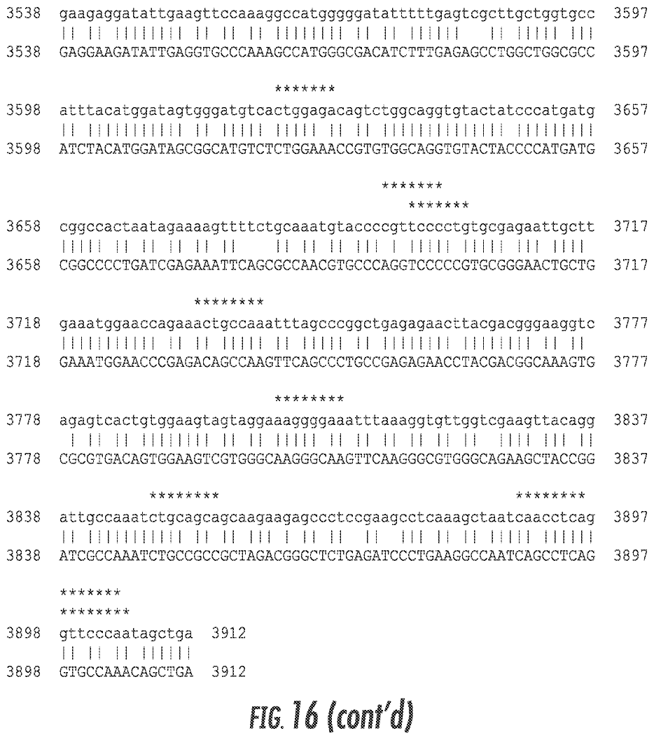

The locations of the miRNA targets in SEQ ID NO: 20 for the miRNAs in Table 2 are also summarized in FIG. 16. Exemplary nucleotide substitutions that can be introduced include substitutions in one or more of nucleotides 571-578, 778-784, 1784-1791, 1892-1899, and 3282-3289 of SEQ ID NO: 20, wherein the one or more nucleotide substitutions reduce or eliminate regulation of expression of an mRNA transcribed from SEQ ID NO: 20 by a member of the let-7 family of miRNAs.

TABLE 2
Exemplary miRNAs and Their Targets in SEQ ID NO: 20

| miRNA | Positions* | Positions* | Positions* | Positions* | Positions* |
|---|---|---|---|---|---|
| mmu-let-7a-5p; | 572-578 | 778-784 | 1784-1791 | 1892-1899 | 3282-3289 |
| mmu-let-7b-5p; | | | | | |
| mmu-let-7c-5p; | | | | | |
| mmu-let-7d-5p; | | | | | |
| mmu-let-7e-5p; | | | | | |
| mmu-let-7f-5p; | | | | | |
| mmu-let-7g-5p; | | | | | |
| mmu-let-7i-5p; | | | | | |
| mmu-let-7k; | | | | | |
| mmu-miR-98-5p | | | | | |
| mmu-miR-1961 | | | | | |
| mmu-miR-7232-3p | 1091-1097 | 2559-2566 | | | |
| mmu-miR-6375 | 150-156 | | | | |
| mmu-miR-28a-5p | 90-97 | | | | |
| mmu-miR-708-5p | | | | | |
| mmu-miR-5134-5p | 147-154 | 3734-3740 | | | |
| mmu-miR-377-5p | 3889-3896 | | | | |
| mmu-miR-672-5p | | | | | |
| mmu-miR-7227-5p | 2626-2633 | 2732-2738 | | | |
| mmu-miR-666-3p | 3848-3855 | | | | |
| mmu-miR-7243-5p | 1449-1456 | | | | |
| mmu-miR-3089-5p | 492-499 | | | | |
| mmu-miR-7657-3p | 1735-1741 | 3489-3496 | | | |
| mmu-miR-383-3p | 235-242 | | | | |
| mmu-miR-6418-5p | 1027-1034 | 1621-1627 | 3697-3703 | | |
| mmu-miR-7211-5p | 289-296 | 1168-1174 | 1214-1220 | 2760-2766 | |
| mmu-miR-1190 | 1486-1493 | | | | |
| mmu-miR-19a-3p | 2138-2144 | 2785-2791 | | | |
| mmu-miR-19b-3p | | | | | |
| mmu-miR-3069-5p | 148-154 | 1363-1369 | 3733-3740 | | |
| mmu-miR-143-3p | 1463-1470 | | | | |
| mmu-miR-12198-3p | 1030-1037 | 1854-1861 | | | |
| mmu-miR-701-3p | 46-53 | 2500-2506 | | | |
| mmu-miR-5709-3p | 684-690 | 1128-1135 | | | |
| mmu-miR-7093-3p | 1873-1880 | 1900-1906 | 2583-2589 | | |
| mmu-miR-212-5p | 1733-1740 | | | | |
| mmu-let-7j | 571-578 | 1785-1791 | 1893-1899 | 3283-3289 | |
| mmu-miR-7080-5p | 91-98 | | | | |
| mmu-miR-101b-3p | 1504-1511 | 2201-2207 | | | |
| mmu-miR-7679-3p | 2679-2686 | | | | |
| mmu-miR-8116 | | | | | |
| mmu-miR-503-3p | 3225-3232 | | | | |
| mmu-miR-20a-3p | 98-105 | 3104-2110 | | | |
| mmu-miR-1928 | 980-986 | | | | |
| mmu-miR-291b-3p | 2140-2147 | | | | |
| mmu-miR-350-5p | | | | | |
| mmu-miR-103-3p | 1535-1542 | | | | |
| mmu-miR-107-3p | | | | | |
| mmu-miR-1197-5p | 1091-1097 | 2560 | | | |
| mmu-miR-1964-5p | 801-808 | 1272 | | | |
| mmu-miR-6949-3p | 1165-1172 | 3802-3809 | | | |
| mmu-miR-758-5p | 1091-1097 | 2560-2566 | | | |
| mmu-miR-6950-5p | 351-357 | 3193-3199 | | | |
| mmu-miR-5128 | 151-157 | | | | |
| mmu-miR-3100-5p | 3898-3905 | | | | |
| mmu-miR-6341 | 732-738 | 2139-2145 | | | |
| mmu-miR-1247-3p | 3694-3700 | 3898-3905 | | | |
| mmu-miR-7033-5p | 608-614 | 2076-2082 | 2469-2475 | 3622-3628 | |
| mmu-miR-380-5p | 1091-1097 | 1777-1783 | 2560-2566 | | |
| mmu-miR-3071-3p | 330-336 | 489-495 | 3449-3455 | | |
| mmu-miR-202-3p | 571-578 | 1785-1791 | 1893-1899 | 3283-3289 | |
| mmu-miR-1911-3p | 1624-1630 | 19261-1933 | | | |
| mmu-miR-804 | 157-163 | | | | |
| mmu-miR-677-5p | 3116-3122 | 3233-3239 | | | |
| mmu-miR-324-5p | 2660-2667 | | | | |
| mmu-miR-3075-3p | 1734-1741 | 3490-3496 | | | |
| mmu-miR-673-5p | 236-242 | 1592-1598 | 2273-2279 | | |

*corresponding nucleotide positions in SEQ ID NO: 20

Thus, in some embodiments the nucleotide sequences of the presently disclosed subject matter comprise one or more nucleotide substitutions in one or more of the nucleotide nucleotide positions of SEQ ID NO: 20 identified in Table 2, optionally in one or more of nucleotide position ranges 571-578, 778-784, 1784-1791, 1892-1899, and 3282-3289 of SEQ ID NO: 20, and further wherein the one or more nucleotide substitutions reduce or eliminate regulation of expression of an mRNA transcribed from SEQ ID NO: 20 by a member of an miRNA family listed in Table 2, optionally a member of the let-7 family of miRNAs, and further wherein the one or more nucleotide substitutions is/are silent with respect to the amino acid encoded by a codon comprising the one or more nucleotide substitutions as compared to the corresponding codon in SEQ ID NO: 20. In some embodiments, a nucleotide sequence of the presently disclosed subject matter thus encodes SEQ ID NO: 23 and comprises, consists essentially of, or consists of SEQ ID NO: 22. Since SEQ ID NO: 22 is a nucleotide sequence that encodes a Δhel-DICER1 polypeptide of the presently disclosed subject matter, this nucleotide sequence is also referred to herein as a “Δhel-DICER1 coding cassette” or simply a “Δhel-DICER1 cassette”.

Alternatively or in addition, a nucleotide sequence of the presently disclosed subject matter encodes a polypeptide comprising, consisting essentially of, or consisting of the amino acid sequence set forth in SEQ ID NO: 23 and that in some embodiments at least 50%, 55%, 60%, 65%, 70%, 75%, 80%, 85%, 90%, 95%, 96%, 97%, 98%, or 99% percent identical to SEQ ID NO: 20 or SEQ ID NO: 22, wherein the polypeptide is at least 90%, 95%, 96%, 97%, 98%, or 99% percent identical to SEQ ID NO: 23.

The terms “identical” or percent “identity” in the context of two or more nucleotide or polypeptide sequences, refer to two or more sequences or subsequences that are the same or have a specified percentage of amino acid residues or nucleotides that are the same, when compared and aligned for maximum correspondence, as measured using one of the sequence comparison algorithms disclosed herein or by visual inspection. For sequence comparison, typically one sequence acts as a reference sequence to which test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters. In some embodiments, a percent identity is calculated over the full length of one or both of the two sequences being compared.

Optimal alignment of sequences for comparison can be conducted, e.g., by the local homology algorithm of Smith & Waterman, 1981; by the homology alignment algorithm of Needleman & Wunsch, 1970; by the search for similarity method of Pearson & Lipman, 1988; by computerized implementations of these algorithms (e.g., GAP, BESTFIT, FASTA, and TFASTA), or by visual inspection. See generally, Ausubel et al., 1992.

An exemplary algorithm for determining percent sequence identity and sequence similarity is the BLAST algorithm, which is described in Altschul et al., 1990. Software for performing BLAST analyses is publicly available through the website of the United States National Center for Biotechnology Information (NCBI). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength W=11, an expectation E=10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See Henikoff & Henikoff, 1992.

In addition to calculating percent sequence identity, the BLAST algorithm also performs a statistical analysis of the similarity between two sequences. See e.g., Karlin & Altschul, 1993. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in some embodiments less than about 0.1, in some embodiments less than about 0.01, and in some embodiments less than about 0.001.

In some embodiments, the nucleotide sequences of the presently disclosed subject matter are present in a vector. Vectors can be designed to replicate a nucleotide sequence of the presently disclosed subject matter in a cell, optionally a prokaryotic or a eukaryotic cell, and exemplary vectors that are useful for these purposes are known to one of ordinary skill in the art.

Additionally, a vector of the presently disclosed subject matter can be employed for expressing a polypeptide encoded thereby, in some embodiments a polypeptide of the presently disclosed subject matter, in a host cell. In such an embodiment, the vector can be referred to as an expression vector. Depending on the cell and the nature of the expression desired, various expression vectors are also known to one of ordinary skill in the art.

In some embodiments, the expression vector is designed to express a polypeptide of the presently disclosed subject matter in a human cell after introduction of the vector into the cell or into a location where the expression vector can accumulate in the cell. In some embodiments, the virus is selected from adeno-associated virus (AAV), helper-dependent adenovirus, retrovirus, herpes simplex virus, lentivirus, poxvirus, hemagglutinatin virus of Japan-liposome (HVJ) complex, Moloney murine leukemia virus, and HIV-based virus. In some embodiments, the AAV capsid or inverted terminal repeats (ITRs) is selected from the group consisting of: AAV1, AAV2, AAV3, AAV4, AAV5, AAV6, AAV7, AAV8, AAV9, AAV10, AAV11, AAV12, rh10, and hybrids thereof.

In some embodiments the vector is an AAV vector. AAV vectors are well known for use in expressing recombinant nucleic acids in cells including human cells. For example U.S. Patent Application Publication Nos. 2019/0000991 and 2019/0008909 (each of which is incorporated by reference in its entirety) discloses compositions and methods for AAV-based gene therapy in humans. See also U.S. Pat. Nos. 8,809,058; 9,540,659; 9,701,984; 9,840,719; 10,214,572; 10,392,632; and U.S. Patent Application Publication Nos. 2008/0206812; 2017/0157267; 2018/0311290; 2019/0002916; 2019/0048362; 2019/0060489.

Limitations of AAV vectors include inefficient production methods, packaging size constraints (introduced gene no larger than 4.5 kb), and a high level of immunity to AAV among adults (although AAV infection is not associated with any disease). The first AAV vectors were produced by transfection of 293 cells with two plasmids (an AAV vector plasmid and an AAV helper plasmid), and infection with adenovirus (reviewed in Muzyczka, 1992). This method provided the essential elements needed for AAV vector production, including AAV terminal repeat (TR) sequences flanking a gene of interest, AAV helper functions consisting of the rep and cap genes, and adenovirus genes.

Improvements to the basic method have included: delivery of adenovirus genes by transfection to eliminate contaminating adenovirus (Grimm et al., 1998; Matsushita et al., 1998; Xiao et al., 1998); delivery of AAV vector sequences within an Ad/AAV hybrid vector to increase vector production (Gao et al., 1998; Liu et al., 1999); and construction of first generation packaging cell lines containing the AAV rep and cap genes (Yang et al., 1994; Clark et al., 1995; Tamayose et al., 1996; Gao et al., 1998; Inoue & Russell, 1998; Liu et al., 1999).

In some embodiments, the viral vector of the presently disclosed subject matter can be measured as pfu (plaque forming units). In some embodiments, the pfu of recombinant virus, or viral vector of the compositions and methods of the presently disclosed subject matter can be about 10⁸ to about 5×10¹⁰ pfu. In some embodiments, recombinant viruses of this disclosure are at least about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, and 5×10¹⁰ pfu. In some embodiments, recombinant viruses of this disclosure are at most about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, and 5×10¹⁰ pfu.

In some embodiments, the viral vector of the presently disclosed subject matter can be measured as vector genomes. In some embodiments, recombinant viruses of this disclosure are 1×10¹⁰ to 3×10¹² vector genomes. In some embodiments, recombinant viruses of this disclosure are 1×10⁹ to 3×10¹³ vector genomes. In some embodiments, recombinant viruses of this disclosure are 1×10⁸ to 3×10¹⁴ vector genomes. In some embodiments, recombinant viruses of the disclosure are at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ vector genomes. In some embodiments, recombinant viruses of this disclosure are 1×10⁸ to 3×10¹⁴ vector genomes. In some embodiments, recombinant viruses of the disclosure are at most about 1×10¹, 1×10², 1×10³, 1×10⁴, 1×10⁵, 1×10⁶, 1×10⁷, 1×10⁸, 1×10⁹, 1×10¹°, 1×10¹¹, 1×10¹², 1×10¹³, 1×10¹⁴, 1×10¹⁵, 1×10¹⁶, 1×10¹⁷, and 1×10¹⁸ vector genomes.

In some embodiments, the viral vector of the presently disclosed subject matter can be measured using multiplicity of infection (MOI). In some embodiments, MOI may refer to the ratio, or multiple of vector or viral genomes to the cells to which the nucleic may be delivered. In some embodiments, the MOI may be 1×10⁶. In some embodiments, the MOI may be 1×10⁵-1×10⁷. In some embodiments, the MOI may be 1×10⁴-1×10⁸. In some embodiments, recombinant viruses of the disclosure are at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI. In some embodiments, recombinant viruses of this disclosure are 1×10⁸ to 3×10¹⁴ MOI. In some embodiments, recombinant viruses of the disclosure are at most about 1×10¹, 1×10², 1×10³, 1×10⁴, 1×10⁵, 1×10⁶, 1×10⁷, 1×10⁸, 1×10⁹, 1×10¹⁰, 1×10¹¹ 1×10¹²1×10¹³, 1×10¹⁴, 1×10¹⁵, 1×10¹⁶, 1×10¹⁷, and 1×10¹⁸ MOI.

In some embodiments the nucleic acid may be delivered without the use of a virus (i.e. with a non-viral vector), and may be measured as the quantity of nucleic acid.

Generally, any suitable amount of nucleic acid may be used with the compositions and methods of this disclosure. In some embodiments, nucleic acid may be at least about 1 pg, 10 pg, 100 pg, 1 pg, 10 pg, 100 pg, 200 pg, 300 pg, 400 pg, 500 pg, 600 pg, 700 pg, 800 pg, 900 pg, 1 μg, 10 μg, 100 μg, 200 μg, 300 μg, 400 μg, 500 μg, 600 μg, 700 μg, 800 μg, 900 μg, 1 ng, 10 ng, 100 ng, 200 ng, 300 ng, 400 ng, 500 ng, 600 ng, 700 ng, 800 ng, 900 ng, 1 mg, 10 mg, 100 mg, 200 mg, 300 mg, 400 mg, 500 mg, 600 mg, 700 mg, 800 mg, 900 mg 1 g, 2 g, 3 g, 4 g, or 5 g. In some embodiments, nucleic acid may be at most about 1 pg, 10 pg, 100 pg, 1 pg, 10 pg, 100 pg, 200 pg, 300 pg, 400 pg, 500 pg, 600 pg, 700 pg, 800 pg, 900 pg, 1 μg, 10 μg, 100 μg, 200 μg, 300 μg, 400 μg, 500 μg, 600 μg, 700 μg, 800 μg, 900 μg, 1 ng, 10 ng, 100 ng, 200 ng, 300 ng, 400 ng, 500 ng, 600 ng, 700 ng, 800 ng, 900 ng, 1 mg, 10 mg, 100 mg, 200 mg, 300 mg, 400 mg, 500 mg, 600 mg, 700 mg, 800 mg, 900 mg, 1 g, 2 g, 3 g, 4 g, or 5 g.

In some embodiments, a self-complementary vector (sc) may be used. The use of self-complementary AAV vectors may bypass the requirement for viral second-strand DNA synthesis and may lead to greater rate of expression of the transgene protein, as provided by Wu, Hum Gene Ther. 2007, 18(2):171-82, incorporated by reference herein.

In some embodiments, several AAV vectors may be generated to enable selection of the most optimal serotype, promoter, and transgene.

In some embodiments, the vector can be a targeted vector, especially a targeted vector that selectively binds to a specific cell, such as cancer cells or tumor cells or eye cells. Viral vectors for use in the disclosure can include those that exhibit low toxicity to a target cell and induce production of therapeutically useful quantities of the anti-VEGF protein in a cell specific manner.

The compositions and methods of the disclosure provide for any suitable viral nucleic acid delivery systems including but not limited to use of at least one of an adeno-associated virus (AAV), adenovirus, helper-dependent adenovirus, retrovirus, herpes simplex virus, lentivirus, poxvirus, hemagglutinatin virus of Japan-liposome (HVJ) complex, Moloney murine leukemia virus, and HIV-based virus. Preferably, the viral vector comprises a strong eukaryotic promoter operably linked to the polynucleotide e.g., a cytomegalovirus (CMV) promoter.

Generally, any suitable viral vectors may be engineered to be optimized for use with the compositions and methods of the disclosure. For example, viral vectors derived from adenovirus (Ad) or adeno-associated virus (AAV) may be used. Both human and non-human viral vectors can be used and the recombinant viral vector can be altered such that it may be replication-defective in humans. Where the vector is an adenovirus, the vector can comprise a polynucleotide having a promoter operably linked to a gene encoding the anti-VEGF protein and is replication-defective in humans.

To combine advantageous properties of two viral vector systems, hybrid viral vectors may be used to deliver a nucleic acid encoding a sFLT-1 protein to a target cell or tissue. Standard techniques for the construction of hybrid vectors are well-known to those skilled in the art. Such techniques can be found, for example, in Sambrook, et al., In Molecular Cloning: A laboratory manual. Cold Spring Harbor, N.Y. or any number of laboratory manuals that discuss recombinant DNA technology. Double-stranded AAV genomes in adenoviral capsids containing a combination of AAV and adenoviral ITRs may be used to transduce cells. In another variation, an AAV vector may be placed into a “gutless”, “helper-dependent” or “high-capacity” adenoviral vector. Adenovirus/AAV hybrid vectors are discussed in Lieber et al., J. Virol. 73:9314-9324, 1999. Retrovirus/adenovirus hybrid vectors are discussed in Zheng et al., Nature Biotechnol. 18:176-186, 2000.

Retroviral genomes contained within an adenovirus may integrate within the target cell genome and effect stable gene expression.

Replication-defective recombinant adenoviral vectors can be produced in accordance with known techniques. See, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992).

Additionally preferred vectors may include but are not limited to viral vectors, fusion proteins and chemical conjugates. Retroviral vectors include Moloney murine leukemia viruses and HIV-based viruses. In some embodiments a HIV-based viral vector may be used, wherein the HIV-based viral vector comprises at least two vectors wherein the gag and pol genes are from an HIV genome and the env gene is from another virus. DNA viral vectors may be used. These vectors include pox vectors such as orthopox or avipox vectors, herpesvirus vectors such as a herpes simplex I virus (HSV) vector [Geller, A. I. et al., J. Neurochem, 64: 487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A.: 90 7603 (1993); Geller, A. I., et al., Proc Natl. Acad. Sci USA: 87:1149 (1990)], Adenovirus Vectors [LeGal LaSalle et al., Science, 259:988 (1993); Davidson, et al., Nat. Genet. 3: 219 (1993); Yang, et al., J. Virol. 69: 2004 (1995)] and Adeno-associated Virus Vectors [Kaplitt, M. G., et al., Nat. Genet. 8:148 (1994)], incorporated by reference herein.

As such, in some embodiments an AAV vector of the presently disclosed subject matter comprises a Δhel-DICER1 coding cassette as disclosed herein.

The presently disclosed subject matter also provides in some embodiments host cells that comprise a nucleotide sequence of the presently disclosed subject matter, which in some embodiments comprises, consists essentially of, or consists of a Δhel-DICER1 coding cassette.

II.B. Pharmaceutical Compositions

In some embodiments, the nucleotide sequences and/or vectors of the presently disclosed subject matter are provided as part of a pharmaceutical composition. As used herein, the term “pharmaceutical composition” refers to a composition comprising at least one active ingredient (e.g., a nucleotide sequence of the presently disclosed subject matter and/or a polypeptide encoded thereby), whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

In some embodiments, a pharmaceutical composition of the presently disclosed subject matter comprises, consists essentially of, or consists of a nucleotide sequence and/or vector of the presently disclosed subject matter and/or a polypeptide encoded thereby and a pharmaceutically acceptable diluent and/or excipient. As used herein, the term “pharmaceutically acceptable” refers to physiologically tolerable, for either human or veterinary application. Similarly, “pharmaceutical compositions” include formulations for human and veterinary use. The term “pharmaceutically acceptable carrier” also refers to a chemical composition with which an appropriate compound or derivative can be combined and which, following the combination, can be used to administer the appropriate compound to a subject. In some embodiments, a pharmaceutically acceptable diluent and/or excipient is pharmaceutically acceptable for use in a human.

In some embodiments, the pharmaceutical compositions of the presently disclosed subject matter are for use in preventing and/or treating a disease or disorder associated with undesirably low DICER1 expression in the eye, optionally the retina, further optionally the RPE, of a subject in need thereof. In some embodiments, the disease or disorder of the eye is associated with RPE degeneration, aberrant choroidal and retinal neovascularization (CRNV), or both. In some embodiments, the effective amount restores undesirably low DICER1 expression in the eye, optionally the retina, of the subject.

The pharmaceutical compositions of the presently disclosed subject matter can in some embodiments consist of the active ingredient alone, in a form suitable for administration to a subject, or the pharmaceutical composition can in some embodiments comprise or consist essentially of the active ingredient and one or more pharmaceutically acceptable carriers, one or more additional ingredients, or some combination of these. The active ingredient can be present in the pharmaceutical composition in the form of a physiologically acceptable ester or salt, such as in combination with a physiologically acceptable cation or anion, as is well known in the art.

As used herein, the term “physiologically acceptable” ester or salt refers to an ester or salt form of the active ingredient which is compatible with any other ingredients of the pharmaceutical composition, which is not deleterious to the subject to which the composition is to be administered.

The formulations of the pharmaceutical compositions described herein can be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts.

II.B.1. Formulations

The compositions of the presently disclosed subject matter thus comprise in some embodiments a composition that includes a carrier, particularly a pharmaceutically acceptable carrier, such as but not limited to a carrier pharmaceutically acceptable in humans. Any suitable pharmaceutical formulation can be used to prepare the compositions for administration to a subject.

For example, suitable formulations can include aqueous and non-aqueous sterile injection solutions that can contain anti-oxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

The therapeutic regimens and compositions of the presently disclosed subject matter can be used with additional adjuvants or biological response modifiers including, but not limited to, cytokines and other immunomodulating compounds.

Controlled- or sustained-release formulations of a pharmaceutical composition of the presently disclosed subject matter can be made using conventional technology. A formulation of a pharmaceutical composition of the invention suitable for oral administration can be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

Liquid formulations of a pharmaceutical composition of the presently disclosed subject matter which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin.

Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose.

Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively).

Known emulsifying agents include, but are not limited to, lecithin and acacia. Known preservatives include, but are not limited to, methyl, ethyl, or n-propyl parahydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil in water emulsion or a water-in-oil emulsion.

The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may also be prepared, packaged, or sold in a formulation suitable for rectal administration, vaginal administration, or parenteral administration.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane dial, for example.

Other acceptable diluents and solvents include, but are not limited to, Ringer's solution, isotonic sodium chloride solution, and fixed oils such as synthetic mono or di-glycerides. Other parentally-administrable formulations which are useful include those which comprise the active ingredient in microcrystalline form, in a liposomal preparation, or as a component of a biodegradable polymer systems.

Compositions for sustained release or implantation may comprise pharmaceutically acceptable polymeric or hydrophobic materials such as an emulsion, an ion exchange resin, a sparingly soluble polymer, or a sparingly soluble salt. Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, can in some embodiments have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985) Remington's Pharmaceutical Sciences, Mack Publishing Co., Easton, Pa., United States of America, which is incorporated herein by reference in its entirety.

II.B.2. Administration

Suitable methods for administration of the compositions of the presently disclosed subject matter include, but are not limited to intravitreous injection; subretinal injection; episcleral injection; sub-Tenon's injection; retrobulbar injection; peribulbar injection; topical eye drop application; release from a sustained release implant device that is sutured to or attached to or placed on the sclera, or injected into the vitreous humor, or injected into the anterior chamber, or implanted in the lens bag or capsule; oral administration, intravenous administration; intramuscular injection; intraparenchymal injection; intracranial administration; intraarticular injection; retrograde ureteral infusion; intrauterine injection; intratesticular tubule injection; and any combination thereof. Exemplary methods for administering compositions to the eye include those described in, for example, U.S. Pat. Nos. 7,745,389; 8,664,176; 9,314,453; 10,004,788; and 10,117,931, each of which is incorporated herein by reference in its entirety.

II.B.3. Dose

An effective dose of a composition of the presently disclosed subject matter is administered to a subject in need thereof. A “treatment effective amount” or a “therapeutic amount” is an amount of a therapeutic composition sufficient to produce a measurable response (e.g., a biologically or clinically relevant response in a subject being treated). Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the therapeutic composition, the route of administration, combination with other drugs or treatments, the severity of the condition being treated, and the condition and prior medical history of the subject being treated. However, it is within the skill of the art to start doses of the compound at levels lower than required to achieve the desired therapeutic effect and to gradually increase the dosage until the desired effect is achieved. The potency of a composition can vary, and therefore a “treatment effective amount” can vary. However, using the assay methods described herein, one skilled in the art can readily assess the potency and efficacy of a candidate compound of the presently disclosed subject matter and adjust the therapeutic regimen accordingly. After review of the disclosure of the presently disclosed subject matter presented herein, one of ordinary skill in the art can tailor the dosages to an individual subject, taking into account the particular formulation, method of administration to be used with the composition, and particular disease treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

### III. Methods of Use

The nucleotide sequences of the presently disclosed subject matter in some embodiments encode Dicer1 polypeptides, more particularly Δhel-DICER1 polypeptides, and in some embodiments are intended for use in expressing the Δhel-DICER1 polypeptide in cells, tissues, and/or organs of subjects. Accordingly, in some embodiments the presently disclosed subject matter relates to methods for expressing Δhel-DICER1 polypeptides in cells, optionally eye cells, further optionally RPE cells by introducing into a cell a nucleotide and/or vector and/or pharmaceutical composition of the presently disclosed subject matter.

Thus, in some embodiments the presently disclosed subject matter relates to uses of the presently disclosed nucleotide sequences and/or vectors and/or pharmaceutical compositions of the presently disclosed subject matter to express Δhel-DICER1 polypeptides in cells, tissues, and/or organs, optionally cells and/or tissues of the eye, further optionally RPE cells. In some embodiments, the cell, tissue, and/or organ is a human cell, tissue, and/or organ, optionally wherein the cell, tissue, and/or organ is present within a human subject.

Additionally, the presently disclosed subject matter relates in some embodiments to methods for preventing and/or treating development of diseases and/or disorders associated with undesirably low DICER1 expression in a cell, tissue, or organ. In some embodiments, the methods related to diseases and/or disorders associated with undesirably low DICER1 expression in the eye, optionally the retina, further optionally the RPE. Although the methods of the presently disclosed subject matter can be employed with respect to preventing and/or treating any diseases and/or disorders associated with undesirably low DICER1 expression, in some embodiments the disease and/or disorder is age-related macular degeneration (AMD). In some embodiments, the disease and/or disorder of the eye is associated with RPE degeneration, aberrant choroidal and retinal neovascularization (CRNV), or both.

As such, in some embodiments the presently disclosed subject matter relates to uses of the nucleotide sequences and/or vectors and/or the pharmaceutical compositions of the presently disclosed subject matter to prevent and/or treat development of diseases and/or disorders of the eye, optionally the retina, further optionally the RPE, wherein the disease or disorder of the eye is associated with undesirably low DICER1 expression. In some embodiments, the disease and/or disorder is age-related macular degeneration (AMD), optionally draft (atrophic) AMD, and optionally wet (neovascular and/or exudative) AMD. In some embodiments, the disease or disorder of the eye is associated with RPE degeneration, aberrant choroidal and retinal neovascularization (CRNV), or both.

In some embodiments, the methods of the presently disclosed subject matter can be used to prevent or treat macular degeneration, including but not limited to AMD. In some embodiments, macular degeneration is characterized by damage to or breakdown of the macula, which in some embodiments, is a small area at the back of the eye. In some embodiments, macular degeneration causes a progressive loss of central sight, but not complete blindness. In some embodiments, macular degeneration is of the dry type, while in some embodiments, it is of the wet type. In some embodiments, the dry type is characterized by the thinning and loss of function of the macula tissue. In some embodiments, the wet type is characterized by the growth of abnormal blood vessels behind the macula. In some embodiments, the abnormal blood vessels hemorrhage or leak, resulting in the formation of scar tissue if untreated. In some embodiments, the dry type of macular degeneration can turn into the wet type. In some embodiments, macular degeneration is age-related, which in some embodiments is caused by an ingrowth of choroidal capillaries through defects in Bruch's membrane with proliferation of fibrovascular tissue beneath the retinal pigment epithelium.

Treatment and/or prevention of AMD using the compositions and methods of the presently disclosed subject matter can be coupled with known methods. For example, the early and intermediate stages of AMD usually start without symptoms. A comprehensive dilated eye exam can detect AMD. The eye exam can include one or more of the following:

1. Visual acuity test. An eye chart measure is used to measure vision at distances.

2. Dilated eye exam. The eye care professional places drops in the eyes to widen or dilate the pupils. This provides a better view of the back of the eye. Using a special magnifying lens, he or she then looks at your retina and optic nerve for signs of AMD and other eye problems.

3. Amsler grid. The eye care professional also may ask you to look at an Amsler grid. Changes in central vision may cause the lines in the grid to disappear or appear wavy, a sign of AMD.

4. Fluorescein angiogram. In this test, which is performed by an ophthalmologist, a fluorescent dye is injected into the subject's arm. Pictures are taken as the dye passes through the blood vessels in the eye. This makes it possible to see leaking blood vessels, which occur in a severe, rapidly progressive type of AMD.

5. Optical coherence tomography. This technique uses light waves, and can achieve very high-resolution images of any tissues that can be penetrated by light such as the eyes.

There are also multiple methods available for predicting susceptibility to age-related macular degeneration or geographic atrophy. As mentioned above, for example, “age-related macular degeneration or geographic atrophy” is not meant to infer that geographic atrophy is not a form or stage of age-related macular degeneration, but that a treatment or diagnosis can be in reference to the two.

Methods and biomarkers are available for predicting whether a subject is susceptible to AMD, including, for example, the existence genetic variants of complement factor H (CFH) and high-temperature requirement factor A-1 (HTRA1) that can be detected, smoking, and, of course, age. When a subject has been tested and is diagnosed or predicted to be susceptible to an RPE disease or disorder, one or more of the therapeutic agents of the presently disclosed subject matter can be administered prophylactically.

Additionally, in some embodiments the presently disclosed subject matter relates to methods for restoring undesirably low DICER1 expression in a cell, tissue, or organ, optionally a cell or tissue of the eye and more particularly the retina of a subject in need thereof. In some embodiments, the nucleotide sequences and/or vectors and/or pharmaceutical compositions of the presently disclosed subject matter can be employed to restore undesirably low DICER1 expression in the eye, optionally the retina, of a subject.

Any method of administration can be employed in order to deliver the compositions of the presently disclosed subject matter to their desired target cell(s), tissue(s), and/or organ(s), which routes of administration include but are not limited to intravitreous injection; subretinal injection; episcleral injection; sub-Tenon's injection; retrobulbar injection; peribulbar injection; topical eye drop application; release from a sustained release implant device that is sutured to or attached to or placed on the sclera, or injected into the vitreous humor, or injected into the anterior chamber, or implanted in the lens bag or capsule; oral administration, intravenous administration; intramuscular injection; intraparenchymal injection; intracranial administration; intraarticular injection; retrograde ureteral infusion; intrauterine injection; intratesticular tubule injection; and any combination thereof.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

Without further description, it is believed that one of ordinary skill in the art can, using the preceding description and the following illustrative EXAMPLES, make and utilize the compounds of the presently disclosed subject matter and practice the methods of the presently disclosed subject matter. The following EXAMPLES therefore particularly point out embodiments of the presently disclosed subject matter and are not to be construed as limiting in any way the remainder of the disclosure.

### Materials and Methods for the Examples

Mice. All experiments involving animals were approved by the University of Virginia Animal Care and Use Committee (ACUC) and in accordance with the Association for Research in Vision and Ophthalmology (ARVO) Statement for the use of Animals in Ophthalmic and Visual Research. Mice were maintained on a constant 12 hour/12 hour light/dark cycle. Water and food were provided ad libitum. Mice were euthanatized with CO2 gas under constant gas flow. C57BL/6J wild type mice were obtained from The Jackson Laboratory, Bar Harbor, Me., United States of America. The Dicer1^{d/d} mouse strain backcrossed to C57BL/6J and the Dicer1^{H/H} were maintained in a heterozygous state, and homozygous wild type and mutant littermates were utilized for experiments. The Dicer1^{d/d} strain was bred to Myd88^{-/-} (The Jackson Laboratory) and Casp1^{-/-}/Casp11^{-/-} (Kuida et al., 1995), a generous gift from Dr. Gabriel Nunez. JR5558 mice (The Jackson Laboratory) were maintained as previously described (Hasegawa et al., 2014; Nagai et al., 2014).

Retinal imaging and angiography. Retinal photographs of dilated mouse eyes were taken with a TRC-50 IX camera (Topcon Medical Systems, Inc., Oakland, N.J., United States of America) linked to a digital imaging system (Sony Electronic, Inc., Tokyo, Japan) or with the Micron IV Retinal Microscope (Phoenix Technology Group, Pleasanton, Calif., United States of America). Spectral domain optical coherence tomography (SD-OCT) was acquired with an OCT2 scan head attached to a Micron IV Retinal Microscope (Phoenix Technology Group). Fluorescein angiograms (FA) was used to measure the incidence and severity of CRNV. In anesthetized mice with dilated eyes, sodium fluorescein (0.1 milliliter of 2.5% solution) was injected into the peritoneum, then eyes imaged with a fluorescent microscopic camera (TTRC-50IX, Topcon Medical Systems, Inc., or Micron IV, Phoenix Technology Group) for up to 10 minutes to monitor dye leakage. Images were graded by a trained operator blinded to the treatment groups. For AAV treatment study, the parameter “FA score” was developed to capture both the number and severity of angiographically active lesions. Lesions were only counted in the area corresponding to the injected site, determined anatomically. For each eye, FA score was calculated as follows:

FA Score=n_{Grade 0 lesions}+2*n_{Grade 1 lesions}+3*n_{Grade 2a lesions}+4*n_{Grade 2b lesions}

Histology, immunohistochemistry, and immunofluorescence. For hematoxylin and eosin staining and immunofluorescence, fresh, unfixed mouse eyes were embedded in Optimal Cutting Temperature Compound (Thermo Fisher Scientific, Waltham, Mass., United States of America), frozen in isopentane precooled by liquid nitrogen, and cryo-sectioned at 10 μm. Immunofluorescent staining was performed with a goat antibody against VE-cadherin (1:50, Santa Cruz Biotechnology, Inc., Dallas, Tex., United States of America). Bound antibody was detected with anti-goat secondary antibody (ThermoFisher).

RNA isolation and real-time quantitative PCR analysis. Tissue was collected and homogenized in TRIZOL (Thermo Fisher Scientific) following the manufacturer's protocol. Total RNA was DNase treated and reverse transcribed using QuantiTect Reverse Transcription Kit (Qiagen). The RT products (cDNA) were amplified by real-time quantitative PCR (Applied Biosystems 7900 HT Fast Real-Time PCR system) with Power SYBR green Master Mix. Oligonucleotide primers specific for mouse genes were as follows:

1. caspase 1 (Casp1):
forward
(SEQ ID NO: 1)
5′-ACCCTCAAGTTTTGCCCTTT-3′;
reverse
(SEQ ID NO: 2)
5′-GATCCTCCAGCAGCAACTTC-3′
2. interleukin Iβ (IL1B):
forward
(SEQ ID NO: 3)
5′-GGGCCTCAAAGGAAAGAATC-3′;
reverse
(SEQ ID NO: 4)
5′-TACCAGTTGGGGAACTCTGC-3′
3. interleukin 18 (IL 18):
forward
(SEQ ID NO: 5)
5′-GACAGCCTGTGTTCGAGGAT-3′;
reverse
(SEQ ID NO: 6)
5′-TGGATCCATTTCCTCAAAGG-3′
4. NLR family, pyrin domain containing
3 (Nlrp3):
forward
(SEQ ID NO: 7)
5′-ATGCTGCTTCGACATCTCCT-3′;
reverse
(SEQ ID NO: 8)
5′-AACCAATGCGAGATCCTGAC
5. 18S rRNA:
forward
(SEQ ID NO: 9)
5′-TTCGTATTGCGCCGCTAGA-3′;
reverse
(SEQ ID NO: 10)
5′-CTTTCGCTCTGGTCCGTCTT-3′
6. Dicer1 spanning exons 24 and 25:
forward
(SEQ ID NO: 18)
5′-CCTTGCGTGGTCAGCATTAGCATT-3′;
reverse
(SEQ ID NO: 19)
5′-TTCTCCTCATCCTCCTCGGATCTC-3′

Oligonucleotide primers spanning exons 24 and 25 (SEQ ID NOs: 18 and 19) to detect Dicer1 abundance in Dicer1^{d/d} mice were utilized as previously described (Otsuka et al., 2007).

The QPCR cycling conditions were 50° C. for 2 minutes, 95° C. for 10 minutes, followed by 40 cycles of a two-step amplification program (95° C. for 15 seconds and 58° C. for 1 minute). At the end of the amplification, melting curve analysis was applied to exclude contamination with unspecific PCR products. Relative expressions of target genes were determined by the 2^{ΔΔCt} method.

Western blotting. Purified retina and RPE protein lysates were obtained using an established protocol (Wei et al., 2016). For RPE, eyes from 3-5 eyes were pooled, constituting one independent observation. Purified RPE isolation was confirmed by the presence of the RPE-specific marker RPE65 and absence of the rod photoreceptor protein Rhodopsin by immunoblotting. Protein concentrations were determined using a bicinchoninic acid assay kit (Thermo Fisher Scientific) with bovine serum albumin as a standard. Proteins (40-100 μg) were run on Tris-glycine gels (Invitrogen Corporation, Carlsbad, Calif., United States of America or Bio-Rad Laboratories, Inc., Hercules, Calif., United States of America) and transferred to PVDF membranes. The transferred membranes were blocked for 1 hour at RT and incubated with antibodies against human and mouse DICER1 (1:500; Bethyl Laboratories, Inc., Montgomery, Tex., United States of America), GAPDH (1:1,000; Abcam, Cambridge, Mass., United States of America, Catalogue No. ab83956), β-Actin, (1:1,000; Abcam, Catalogue No. ab8229) and α-Tubulin (1:1,000; Abcam Catalogue No. ab89984). IR dye-conjugated secondary antibodies were used (1:5,000) for 1 hour at RT. The signal was visualized by Licor Odyssey and densitometry quantified by ImageJ.

In situ Caspase-1 activity. In situ detection of Caspase-1 activity was conducted as described previously (Gelfand et al., 2015). Briefly, unfixed eyes were enucleated and immediately placed in OCT mounting media and snap frozen in isopentane cooled by liquid nitrogen. Unfixed 5 μm thick frozen sections of mouse eyes were incubated with CaspaLux1-E1D2 (Oncoimmunin Inc., Gaithersburg, Md., United States of America) for 40 minutes at 37° C. in a humidified chamber. Afterwards, slides were washed 5 times in PBS. Coverslips were placed on the tissue sections and fluorescent and brightfield images were acquired on a Nikon Eclipse Ti inverted fluorescent microscope.

miRNA preparation. 5′ and 3′ pre-let-7a miRNA constructs were synthesized by Integrated DNA Technologies, Inc. (Coralville, Iowa, United States of America) Annealing and ligation protocols were adapted from Fareh et al., 2016. Briefly, a 20 mixture containing 200 pmol 5′ strand and 100 pmol 3′ strand in TE buffer with 100 mM NaCl was annealed by heating to 95° C. then slowly cooling (−1° C. per 30 seconds) to 25° C. Subsequent ligation was achieved by incubating the annealed substrate with 3 μL T4 RNA ligase (5 U/μL; Ambion, Inc., Austin, Tex., United States of America), 3 μL 0.1% BSA, 5 μl 10×ligation buffer, and 19 μL ultrapure water at 16° C. for 24 hours. RNA was isolated by standard ethanol precipitation and resuspended in 10 μL 2×TBE-urea loading dye (Bio-Rad) and 10 μL ultrapure water. After separation on a Novex 15% TBE-urea gel (Thermo Fisher Scientific), the gel was incubated in GelStar Nucleic Acid Gel Stain 10000×(Lonza Group, Morristown, N.J., United States of America) and visualized on a UVP High Performance UV Transilluminator (Analytik Jena US LLC, Upland, Calif., United States of America). Ligated miRNA was excised from the gel, crushed in a 1.5 mL Eppendorf tube, and incubated in 200 μL 0.3 M NaCl-TE (pH 7.5) overnight. Crushed gel solution was filtered through an EDGE DTR filter column (Edge Bio Systems, Gaithersburg, Marylan, United States of America) and precipitated via standard ethanol precipitation. 5′ sequence: 5′-UGAGGUAGUAGGUUGUAUAGUUU UAGGGUCACACC-3′ (SEQ ID NO: 11); 3′ sequence: 5′-pCACCACUGGGAGAUAACUAUACAAUCUACUGUCCySUUCU-3′ (SEQ ID NO: 12).

In vitro DICER1 tube assay. DICER1 plasmids were transfected into HEK293T cells with Lipofectamine 2000 (Thermo Fisher Scientific) according to manufacturer protocol. Protein was collected after 48 hours as in Park et al., 2011. Briefly, cells were collected in 1 ml lysis buffer (500 mM NaCl, 1 mM EDTA, 20 mM Tris (pH 8.0), 1% Triton X-100) and incubated on ice for 20 minutes. After sonication, cells were centrifuged twice at 16000×g for 10 minutes and supernatant transferred to 1.5 ml Eppendorf tube. 100 μL anti-FLAG M2 magnetic beads were equilibrated according to manufacturer protocol and incubated with protein supernatant overnight on an end-to-end tube rotator at 4° C. Beads were washed three times with lysis buffer and four times with Buffer D (200 mM KCl, 20 mM Tris (pH 8.0), 0.2 mM EDTA). The FLAG-DICER1 was eluted from the beads by competition with 250 μL FLAG peptide (100 μg/mL, Sigma Aldrich, St. Louis, Mo., United States of America). To remove excess FLAG peptide, eluate from the competition was passed through an Amicon 100 kDa cutoff filter (Millipore Sigma, Burlington, Mass., United States of America).

In vitro DICER1 cleavage assay was adapted from (Park et al., 2011). Briefly, reactions were performed in a total volume of 10 μL containing 1 μL 10× DICER reaction buffer (100 mM Tris (pH 8.0), 1 mM EDTA, 1000 mM KCl, 100 mM MgCl₂), 1 purified DICER, 1 μL 10 mM DTT, 0.5 μL recombinant RNase inhibitor (5000 U, Takara Bio USA, Inc., Mountain View, Calif., United States of America), pre-let-7a miRNA (20-40 ng), and ultrapure water. Reactions were incubated for 0-90 minutes on a thermocycler followed by addition of 2×TBE-urea loading dye and separation on a 15% TBE-urea gel. Images were visualized on a Licor Odyssey Fc Imaging System in the 700 channel.

Adeno-associated vector design, production, and delivery. Δhel-DICER1 cDNA was cloned into pAAV-MCS (Agilent Technologies, Santa Clara, Calif., United States of America). The total packaging genome size to 5.0 kb. The indicated plasmids were transfected into HeLa (American Type Culture Collection (ATCC), Manassas, Va., United States of America) and primary human retinal pigmented epithelial cells (hRPE; Lonza), maintained in RtEBM (Lonza) following the manufacturer's instructions. Nucleofection with Basic Epithelial Cells NUCLEOFECTOR® Kit (Lonza) was used for transient plasmid transfection with program U-023. The transfection efficiency was >80% as determined by pMaxGFP transfection with fluorescence microscopy. let-7-resistant DICER1 and OptiDicer were synthesized by GeneArt Gene Synthesis (Thermo Fisher Scientific). Expression of OptiDicer was driven by CMV promoter and contained an SV40 polyadenylation signal in the 3′ end. Production and purification of AAV2-OptiDicer was accomplished by Vigene Biosciences.

Intraocular injections. Subretinal injections and intravitreous injections (1 μL each) were performed with a 35-gauge Exmire microsyringe (Ito Corporation). The VEGF neutralizing antibody B20-4.1.1 or an equivalent mass of isotype antibody, both provided by Genentech (South San Francisco, Calif., United States of America), were delivered by intravitreous injection (0.5-1 μg). 1 microliter of 1.0×10¹¹ viral genomes (vg)/ml (or 10⁸ vg/microliter) of AAV-OptiDicer or AAV2-CMV-null (Vector Biolabs, Malvern, Pa., United States of America) were delivered by subretinal injection.

### Example 1

### Genetic Deficiency of Dicer1 Induces Spontaneous RPE Atrophy and Choroidal and Retinal Neovascularization in Two Independent Mouse Strains

Because loss of DICER1 is implicated in advanced atrophic AMD, whether chronic DICER1 deficiency in mice recapitulated retinal pathologies such as those observed in human AMD was investigated. Global ablation of Dicer1 results in early embryonic lethality in mice (Bernstein et al., 2003; Yang et al., 2005). Developmental or postnatal cell type-specific deletion of Dicer1 in the RPE results in rapid and profound RPE and retinal atrophy (Kaneko et al., 2011; Sundermeier et al., 2017). In contrast, the Dicer1^{Gt(β-geo)Han} mouse line, hereafter referred to as Dicer1^{d/d}, harbors a gene trap insertion in intron 24 of the Dicer1 locus which results in a functional reduction in Dicer1 expression by approximately 80% (Otsuka et al., 2007). The Dicer1^{d/d} line is viable, with susceptibility to viral infections, exacerbated experimental rheumatoid arthritis, and infertility due to insufficient corpus luteal angiogenesis among its reported phenotypes (Otsuka et al., 2007; Otsuka et al., 2008; Ostermann et al., 2012; Ostermann et al., 2015; Alsaleh et al., 2016).

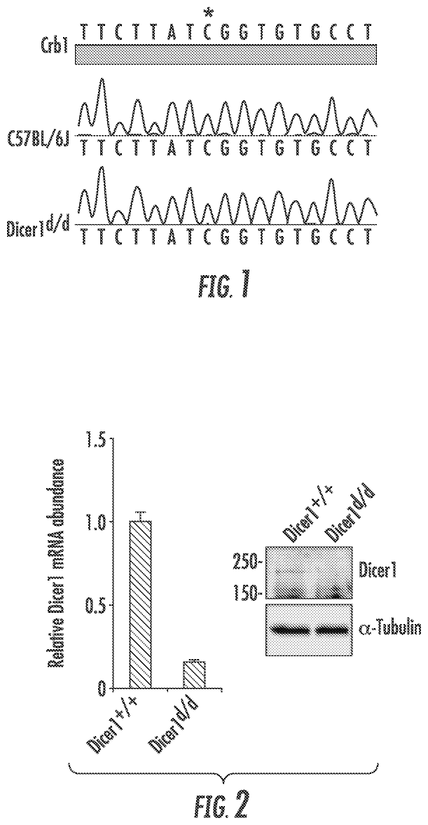

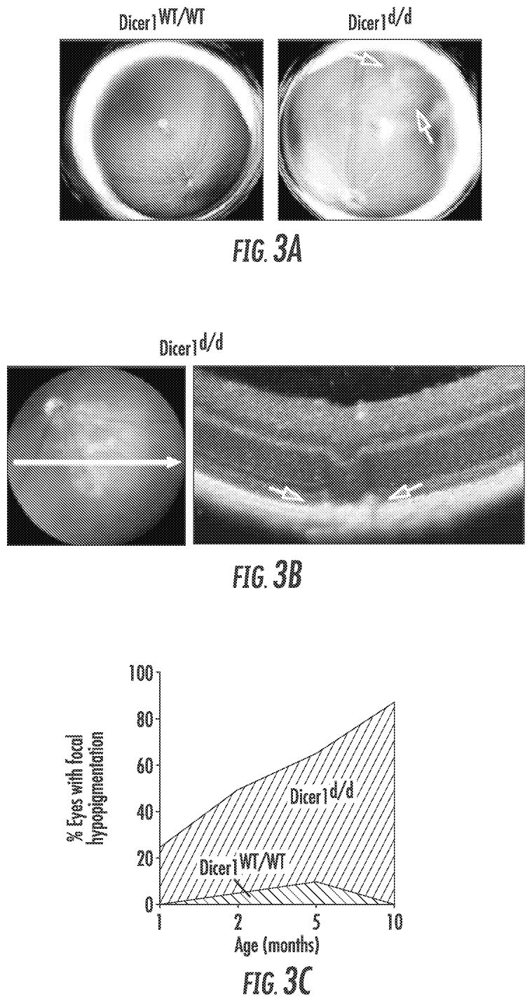

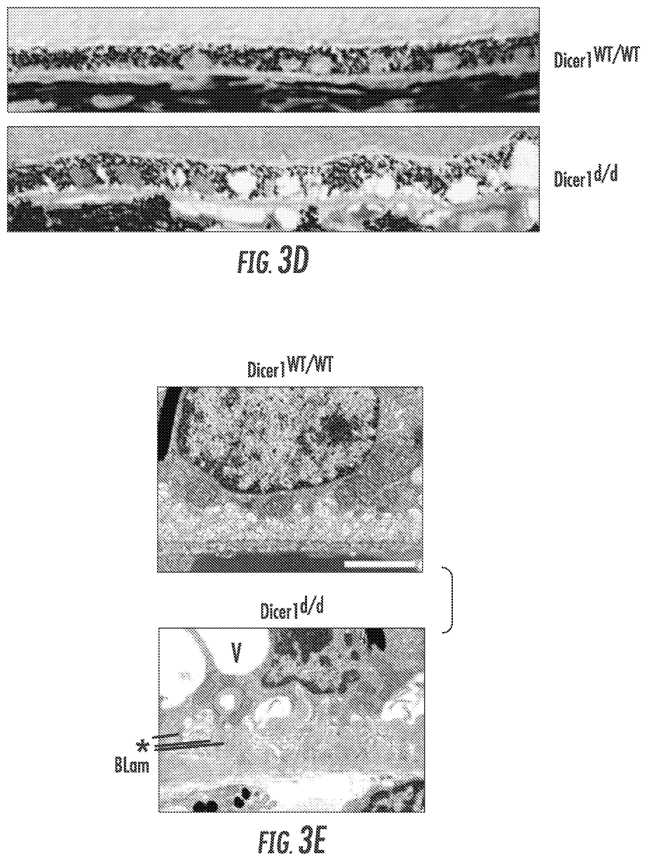

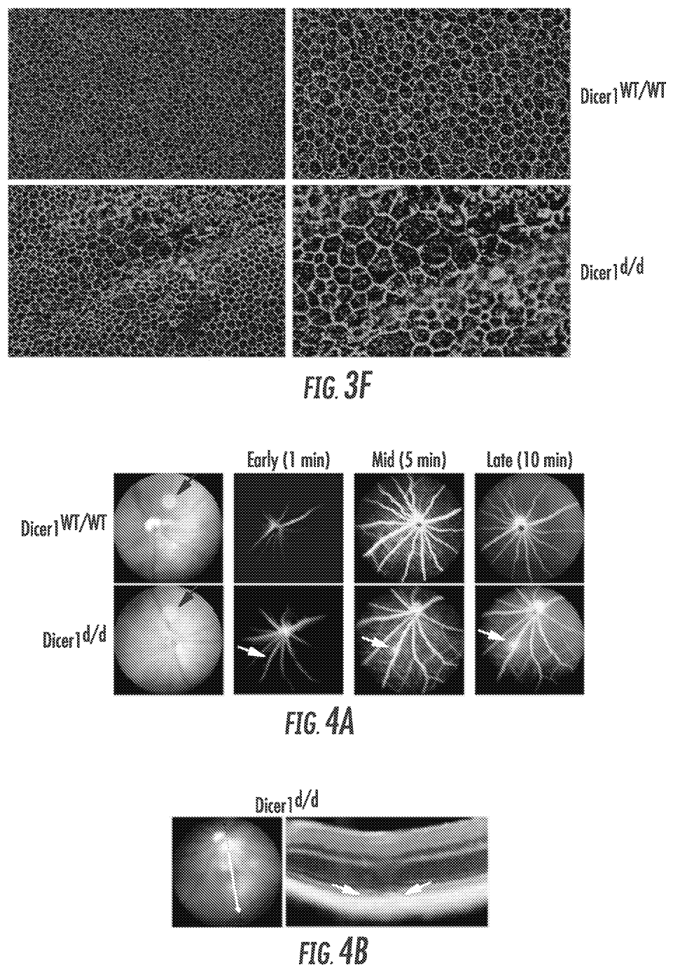

Consistent with its C57BL/6J background, Dicer1^{d/d} did not exhibit hallmark features of the rd8 mutation, a prevalent confounder of retinal phenotypes (Mattapallil et al., 2012). DNA sequencing revealed that Dicer1^{d/d} tested negative for the rd8 mutation (FIG. 1). Consistent with other tissues previously analyzed from this strain, retinal Dicer1 mRNA abundance was reduced by approximately 80% compared to wild type littermate mice (FIG. 2). As expected from prior studies on acute DICER1 deficiency in the RPE, Dicer1^{d/d} mice exhibited spontaneous focal hypo-pigmented patches in fundus retinal images (FIG. 3A). Spectral domain optical coherence tomography (SD-OCT) revealed apically projected hyper-reflective foci in the outer retina and RPE layers (FIG. 3B). The incidence of focal hypopigmentation of the fundus was age-related, with 50% of eyes affected at 8-weeks of age, and increased in frequency up to 75% at ten-months of age (p=0.008 by Spearman rank coefficient test; FIG. 3C). Histological analysis revealed disorganized, hypertrophic, RPE with large vacuoles (FIG. 3D). Ultrastructural analysis of Dicer1^{d/d} retina revealed loose, disorganized RPE basal infoldings and extracellular sub-RPE debris consistent with basal laminar deposits (FIG. 3E), considered to be a general feature of distressed RPE that may have a role in AMD, but that is not specific for human AMD (Curcio, 2018). Hypertrophy, disorganization, and RPE degeneration were also observed by flat mount imaging of the RPE layer (FIG. 3F).

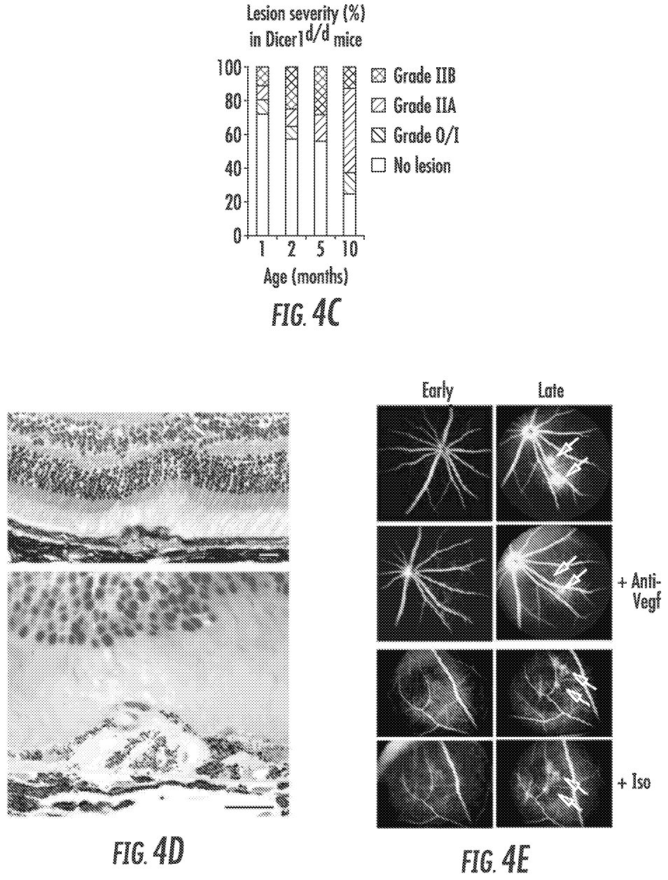

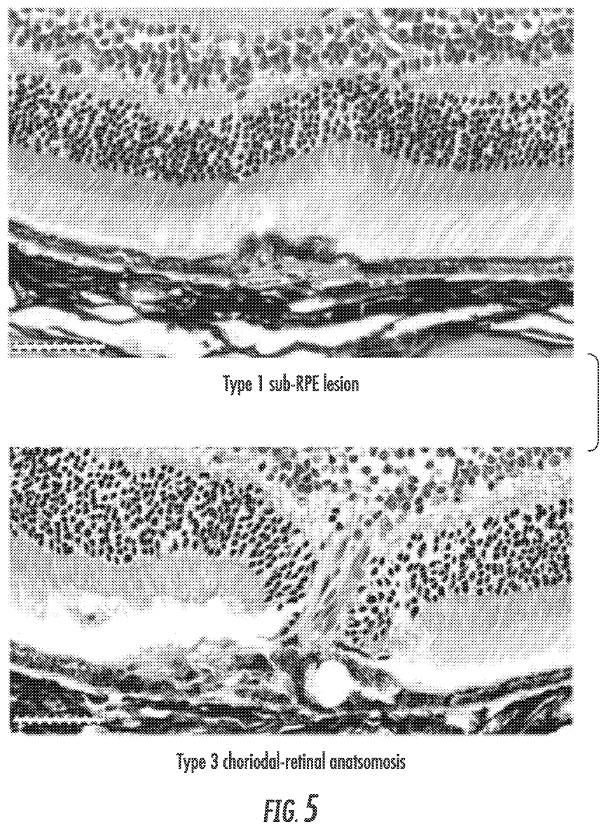

In addition, fluorescein angiography (FA) of Dicer1^{d/d} mice revealed spontaneous hyper-fluorescent foci that expanded over time, consistent with the behavior of immature vessels of active subretinal neovascular lesions (FIG. 4A). Conversely, no angiographically active lesions were observed in any eye from wild type littermate at any age. SD-OCT of Dicer1^{d/d} mice revealed outer retinal discontinuities consistent with choroidal neovascularization (FIG. 4B). The incidence and severity of FA-positive lesions were quantified using an established grading scale (see Yu et al., 2008; Hoerster et al., 2012). Both lesion incidence and severity were significantly associated with age (p<0.001 by Spearman rank coefficient test; FIG. 4C). In the majority of Dicer1^{d/d} mouse eyes harboring angiogenic lesions, most exhibited one discrete lesion, but occasionally more than one lesion was present.

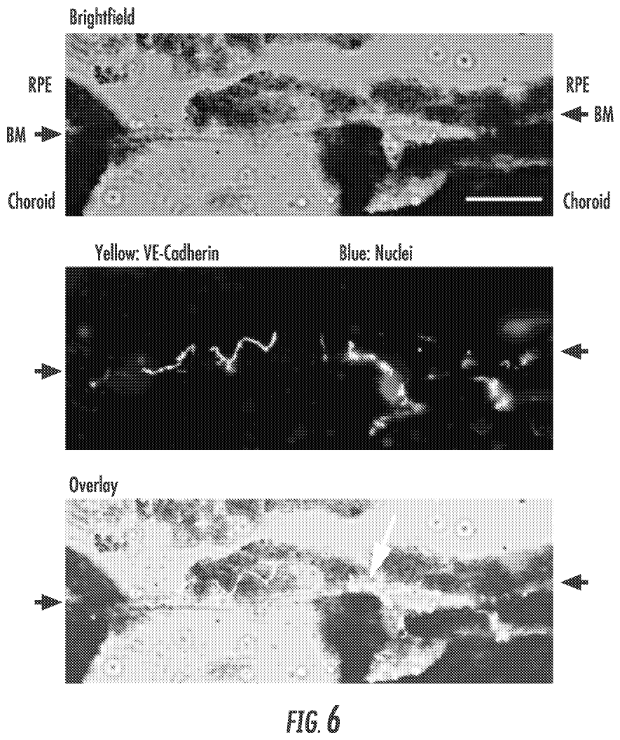

Histological examination revealed type 1 (sub-RPE; FIG. 4D) and type 2 (subretinal) choroidal neovascular (CNV) lesions and type 3 chorioretinal anastomoses in the outer retina (FIG. 5). Vessels were patent with erythrocytes observed surrounded by an intimal layer of endothelial cells. Choroidal endothelial cells were observed traversing Bruch's membrane (FIG. 6). These findings are consistent with CNV in humans and in other experimental models.

Administration of a Vegfa-neutralizing antibody (B20-4.1.1) into the vitreous humor reduced the angiographic activity of neovascular lesions (FIG. 4E), suggesting that neovascularization due to Dicer1 deficiency recapitulated the therapeutic response to anti-VEGFA compounds observed in aberrant neovascularization in human patients.

To more thoroughly evaluate the effect of genetic inhibition of Dicer1 on atrophic and neovascular retinal pathologies, a second Dicer1 hypomorphic mouse strain,

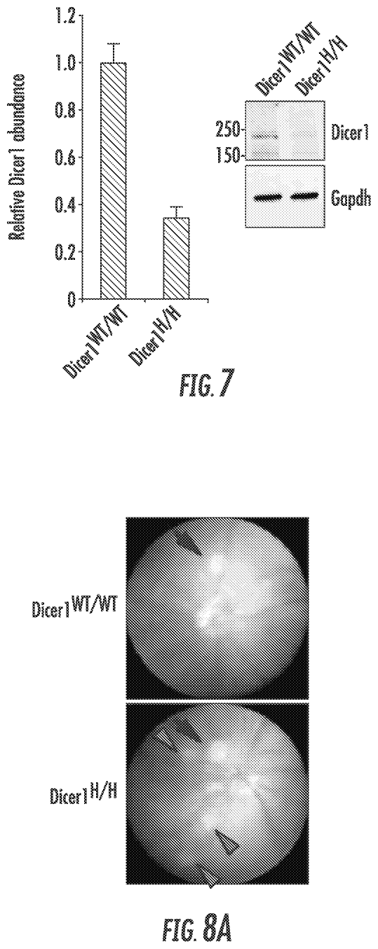

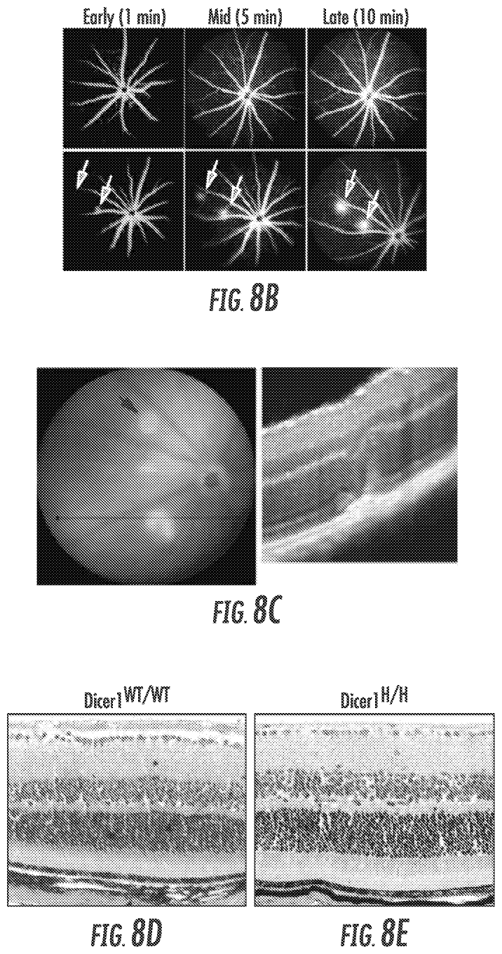

Dicer1^{Gt(RRF266)Byg} (hereafter Dicer1^{H/H}), generated by a different laboratory by inserting a gene trap vector into a different region (intron 22) of the Dicer1 locus and maintained on a different genetic background (Fukasawa et al., 2006; Morita et al., 2009), was investigated. Dicer1 abundance in the retina of Dicer1^{H/H} mice was approximately 65% less than their wild type littermate controls (FIG. 7). Dicer1^{H/H} mice also exhibited spontaneous RPE degeneration, as evidenced by focal hypopigmentation on fundus photography (FIG. 8A) and foci of active neovascular lesions by FA (FIG. 8B), which localized to the subretinal space upon imaging with SD-OCT (FIG. 8C). Histological analysis revealed focal RPE thinning and choroidal neovascularization (FIGS. 8D-8G). Aberrant angiogenic lesions were absent in littermate controls; angiographic leakage was detected in 0/8 eyes Dicer1^{wt/wt} vs. 6/8 eyes Dicer1^{H/H} (p=0.007 by Fisher's exact test). Thus, two independent mouse models of systemic DICER1 deficiency, developed by different laboratories, targeting distinct regions of the Dicer1 locus, and maintained on different genetic backgrounds both exhibited spontaneous RPE atrophy and choroidal neovascularization.

### Example 2

### RPE Degeneration and Aberrant Angiogenesis Due to DICER1 Loss Depends on Innate Immune Signaling

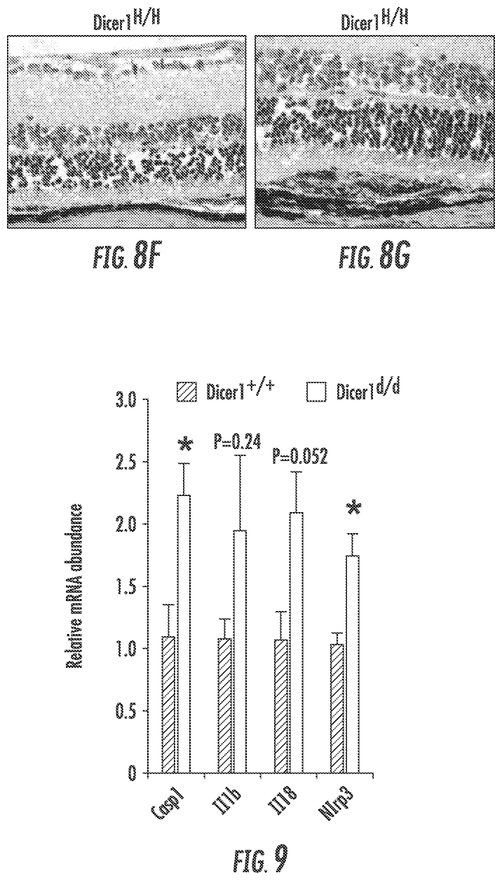

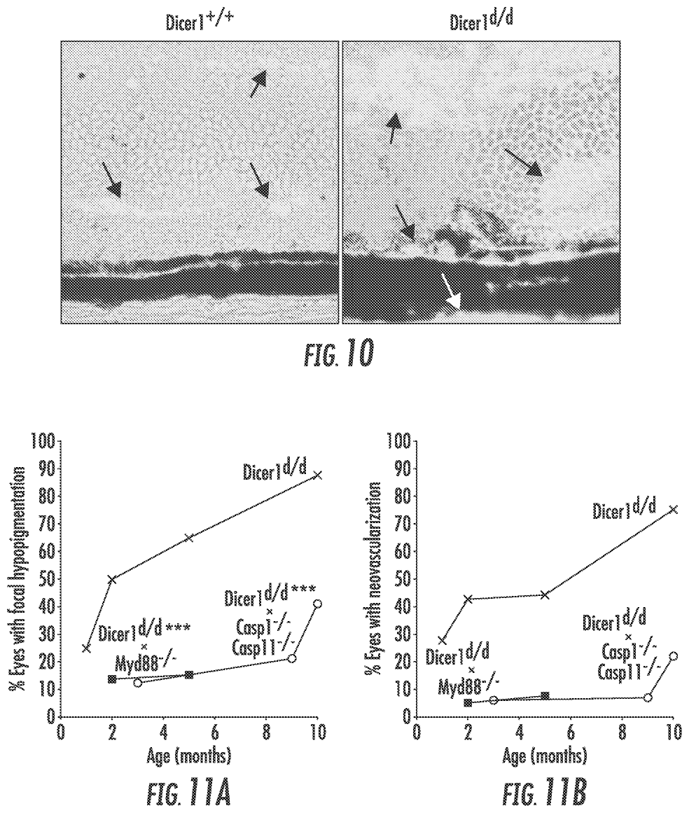

Acute DICER1 antagonism in the RPE promotes activation of the NLRP3 inflammasome, leading to RPE degeneration (Tarallo et al., 2012). The extent to which the NLRP3 inflammasome contributed to RPE atrophy and CNV due to chronic DICER1 deficiency was tested. Inflammasome activity in Dicer1^{d/d} mice was first examined. Transcripts encoding the NLRP3 inflammasome-related genes Casp1 and Nlrp3, and the effector cytokine IL-18 were upregulated in retinas of Dicer1^{d/d} mice compared to littermate controls (FIG. 9). Inflammasome activation, measured by in situ proteolytic activity of a fluorescent Caspase-1 peptide substrate, was also observed in the outer retinae of Dicer1^{d/d} mice in areas of neovascularization (FIG. 10).

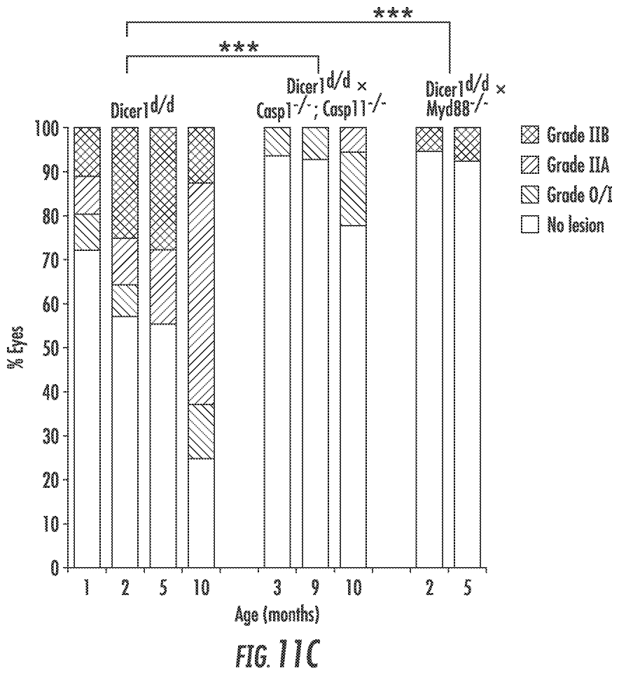

The relationship between DICER1 deficiency and immune signaling constituents in promoting spontaneous retinal pathologies was then ascertained. Dicer1-deficient mice lacking the inflammatory effector caspases-1 and -11 (Dicer1^{d/d}; Casp1^{-/-}; Casp11^{-/-}) exhibited a significantly reduced incidence of focal hypopigmentation compared to caspase-1 and -11 sufficient Dicer1^{d/d} mice (p<0.001 by multinomial logistic regression; FIG. 11A). Moreover, ablation of caspases-1 and -11 also reduced the incidence and severity of pathological neovascular lesions by FA grading (p<0.001; FIGS. 11B and 11C).

The adaptor MyD88, a putative drug target for AMD (Tarallo et al., 2012), transduces several inflammatory stimuli including toll-like receptors (TLR) (excluding TLR3) and receptors for inflammasome effector cytokines IL-1β and IL-18. Dicer1-deficient mice lacking MyD88 (Dicer1^{d/d}; Myd88^{-/-}) also exhibited significantly reduced incidence of focal RPE hypopigmentation (p<0.001; FIG. 11A) and incidence and severity of pathological neovascular lesions (p<0.001; FIG. 11C). Together, these findings indicated that signaling through caspases 1 and 11 and MyD88 mediated both atrophic and neovascular retinal pathologies that arose due to chronic DICER1 deficiency.

### Example 3

### DICER1 Dysregulation in Spontaneous CNV JR5558 Mice

Given the findings that Dicer1 deficiency in mice promotes spontaneous neovascularization, the expression of DICER1 in the JR5558 mouse line, which develops spontaneous CNV (Hasegawa et al., 2014; Nagai et al., 2014) that is dependent on the rd8 mutation in the Crb1 gene locus (Chang et al., 2018), was quantified. Similar to CNV in humans and in Dicer1-deficient mice, neovascular lesions in JR5558 also respond to VEGF neutralization (Nagai et al., 2014; Foxton et al., 2016) and depend on innate immune processes (Nagai et al., 2014; Nagai et al., 2015; Paneghetti & Ng, 2016). Compared to age-matched wild type mice, Dicer1 abundance was significantly reduced in the RPE of JR5558 mice at postnatal days 9-10 (P9-10), coincident with the earliest reported neovascular abnormalities, and reduced DICER1 levels persisted to P28-37 (FIG. 12A). Conversely, Dicer1 abundance in neural retina was elevated compared to age-matched wild type controls when measured in P9-10 and reduced at P28-37 (FIG. 12B). Thus, Dicer1 dysregulation was coincident with the earliest stages of neovascular defects in JR5558 mice, and Dicer1 deficiency in RPE preceded loss in neural retina at later time points, indicating that Dicer1 expression was dysregulated in spontaneous CNV of mice.

### Example 4

### Development of an Exemplary OptiDicer Construct

To determine the functional contribution of Dicer1 deficiency to retinal and choroidal neovascularization in JR5558 mice, a gene therapy strategy capable of restoring Dicer1 activity was developed. Adeno-associated vector (AAV) was selected because this modality has demonstrated safety and efficacy in treating blinding diseases in human patients (Bainbridge et al., 2008; Maguire et al., 2008) and in experimental models of choroidal and retinal neovascularization (Lai et al., 2005; Luo et al., 2013; Sun et al., 2017a; Sun et al., 2017b; Lee et al., 2018; Schnabolk et al., 2018). The human and mouse DICER1 genes are encoded by sequences of 5.7 kb, which is too large to be packaged into a traditional AAV with a size limit of ˜5.2 kb (Dong et al., 1996; Wu et al., 2010). The large N-terminal helicase domain of DICER1 is known to be dispensable for miRNA substrate specificity and processing activity (Ma et al., 2008; Gurtan et al., 2012; Kennedy et al., 2015), and was removed from the coding sequence of the human DICER1, and an initiator methionine codon was added to generate Δhel-DICER1 (SEQ ID NO: 20).

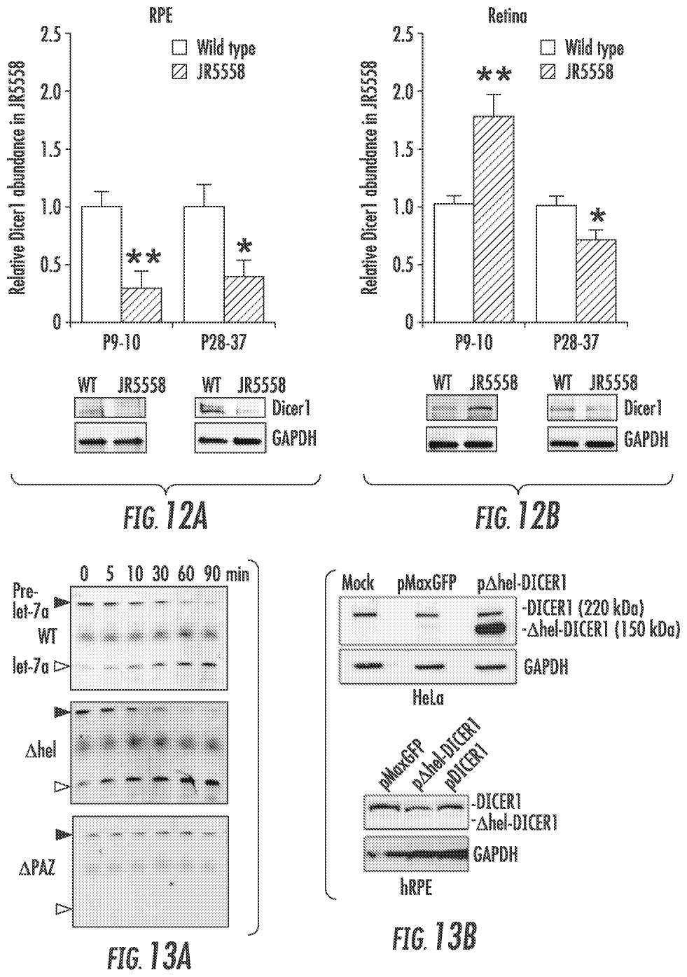

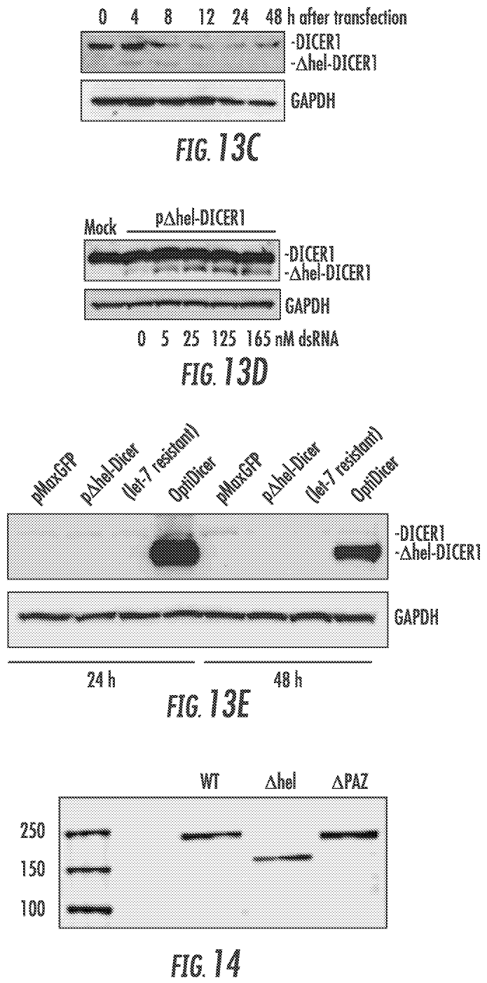

In a tube assay, it was confirmed that purified helicase domain-deleted DICER1 (Δhel-DICER1) retained pre-miRNA processing activity, and that purified DICER1 lacking the PAZ domain necessary for pre-miRNA recognition (ΔPAZ-DICER1) (Ma et al., 2004; MacRae et al., 2007) did not (FIGS. 13A and 14). The coding sequence of Δhel-DICER1 is 3.9 kb, which is compatible with efficient AAV packaging. Δhel-DICER1 cDNA was cloned into pAAV-MCS, with a total packaging genome size, including regulatory elements and AAV inverted terminal repeats (ITR), of 5.0 kb.

Transient transfection of Δhel-DICER1 resulted in robust expression in HeLa cells as detected by immunoblotting (FIG. 13B). However, transfection into human RPE cells resulted in no detectable expression of the truncated protein. Therefore, it was hypothesized that DICER1 expression in RPE was subject to negative autoregulation, potentially arising due to the enhanced miRNA processing activity of DICER1. Consistent with this hypothesis, transient Δhel-DICER1 expression was observed within 4 hours of transfection, but reduced to undetectable levels soon thereafter (FIG. 13C). Further, co-transfection with double-stranded RNA, which can compete with DICER1 processing and RISC loading (Liang et al., 2013), restored Δhel-DICER1 expression to detectable levels (FIG. 13D), indicating that impaired Δhel-DICER1 expression was due to negative feedback via RNA interference.

Because the Δhel-DICER1 insert lacks a native 3′ UTR, it was hypothesized that miRNA binding sites within the coding sequence could be responsible for negative feedback. Forman and colleagues demonstrated that let-7 miRNAs specifically target the DICER1 coding region, identifying three putative target regions that corresponded to nucleotides 3926-3940, 4031-4048, and 5418-5438 in the nucleotide sequence of the human Dicer1 gene product set forth in Accession No. NM_030621.4 of the GENBANK® biosequence database. (Forman et al., 2008). Based on this study, let-7-resistant Δhel-DICER1 with silent mutations of these three targets was generated. Although let-7-resistant Δhel-DICER1 was robustly expressed in HeLa cells, it too failed to express in human RPE cells in detectable levels (FIG. 13E).

Therefore, a pan-miRNA-resistant Δhel-DICER1 was generated. The online miRDB database (http://miRDB.org/miRDB/index.html; see also Wang, 2008; Wong & Wang, 2015; Liu & Wang, 2019) was employed, which identified 44 miRNA seed sequences (37 human and 7 mouse) within the Δhel-DICER1 coding region. 33 of these putative seed sequences (28 human and 5 mouse) were successfully removed by introducing silent mutations (see FIG. 16). Codon optimization was also employed. The resulting construct, referred to herein as “OptiDicer” (SEQ ID NO: 22, which encodes the human DICER1 polypeptide of SEQ ID NO: 23), exhibited robust and stable expression in human RPE cells (FIG. 13E).

### Example 5

### Gene Delivery with an Exemplary OptiDicer Construct Improves Spontaneous Chorioretinal Neovascularization in Mice

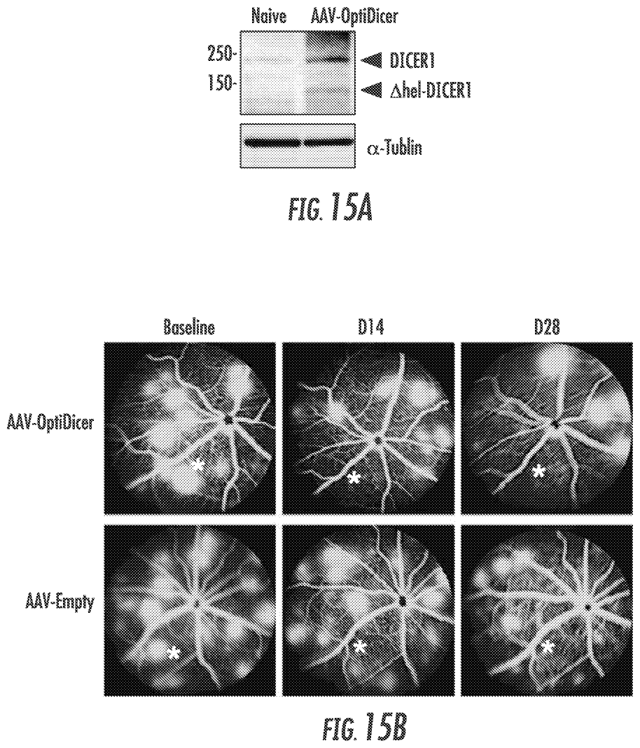

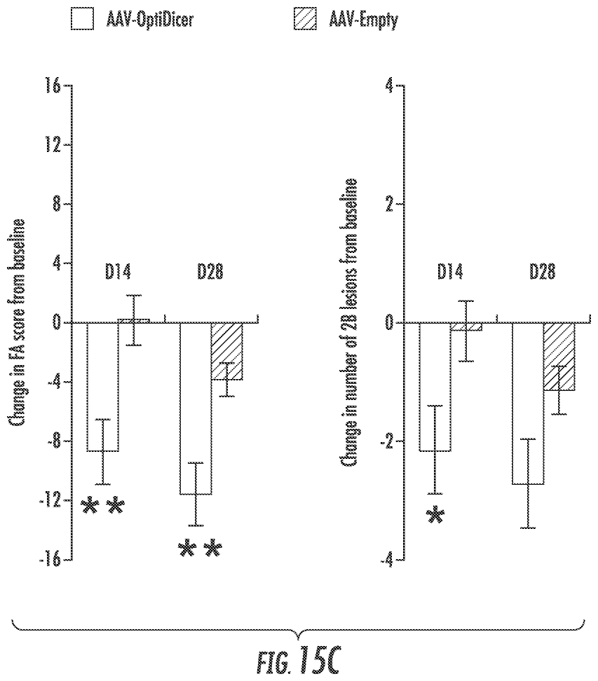

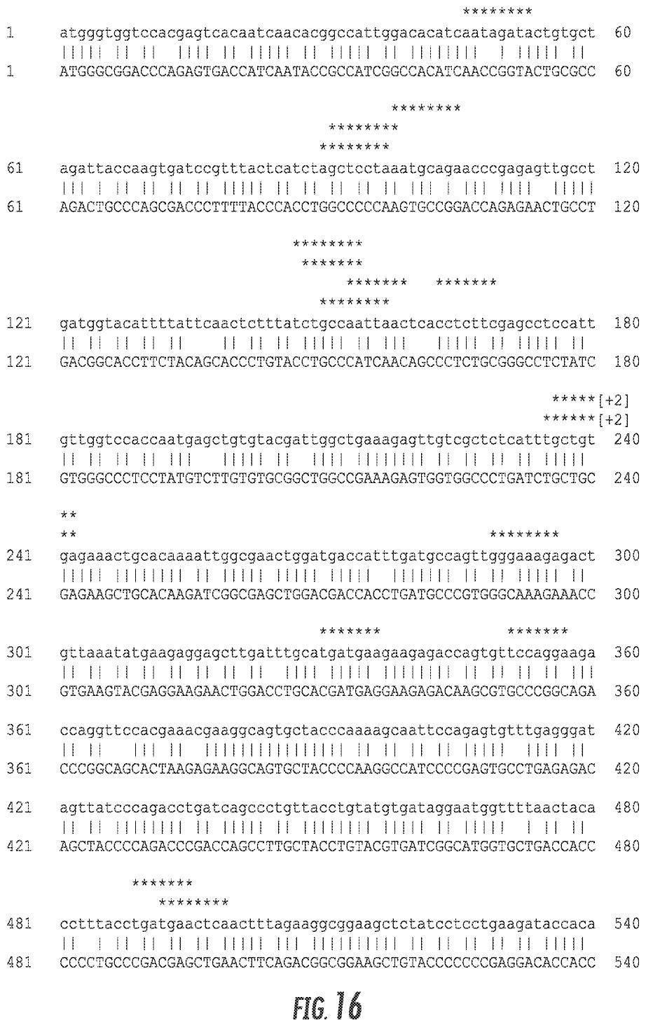

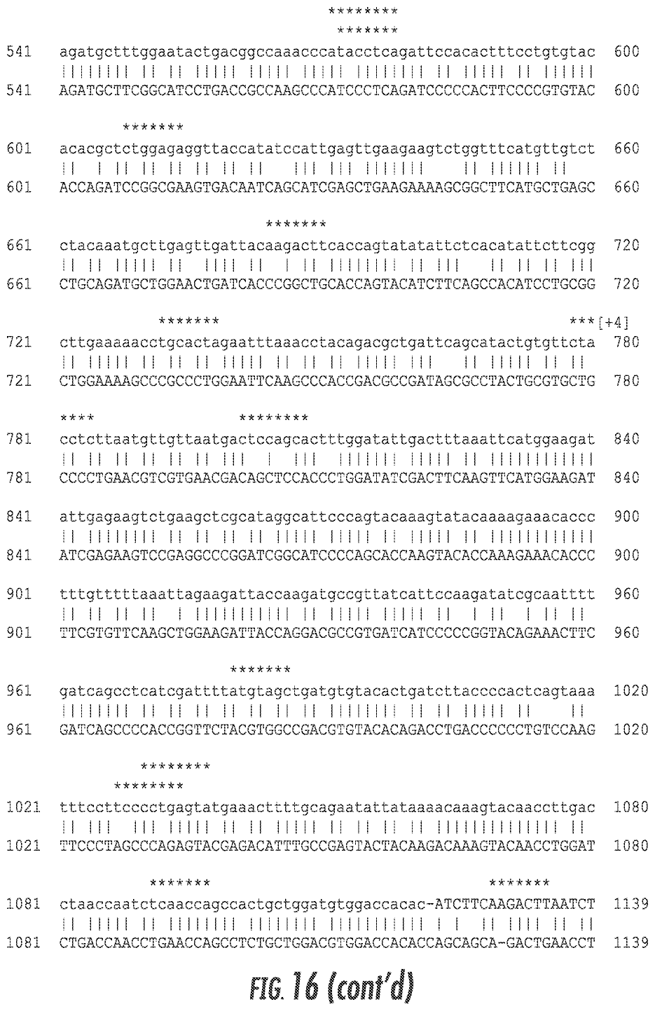

Stable expression of AAV-encoded OptiDicer was confirmed in retina of JR5558 mice (FIG. 15A). To determine whether DICER1 gene delivery affected CRNV in JR5558 mice, first, FA was performed on naïve 6-week old JR5558 mice with established CNV. Then, AAV-OptiDicer or an empty AAV2-control was administered by subretinal injection in contralateral eyes. Fourteen and twenty-eight days after injections, follow-up FA revealed significant improvement in both the frequency and severity of neovascular lesions within injected areas compared to eyes transduced with a control vector (FIGS. 15B and 15C), suggesting that subretinal delivery of a bioactive DICER1 variant by AAV antagonized CRNV in JR5558 mice.

### Discussion of the Examples

Development of the OptiDicer construct. To determine the contribution of DICER1 deficiency to retinal and choroidal neovascular phenotypes in JR5558 mice, a gene therapy approach capable of restoring DICER1 activity was developed as disclosed herein. Wild-type human and mouse DICER1 genes, with coding sequences of 5.7 kb, are too large to package into a traditional AAV, which has a size limit of ˜5.2 kb for infectious virus production. The large N-terminal helicase domain of DICER1 is dispensable for miRNA substrate specificity and processing activity, and a naturally occurring helicase-deficient DICER1 isoform efficiently cleaves mouse SINE RNAs. Thus, the helicase domain is also dispensable for non-canonical DICER1 substrate processing and was excluded from the DICER1 expression construct. In a tube assay, it was confirmed that purified helicase domain-deleted DICER1 (referred to here as “Δhel-DICER1”) cleaved pre-miRNA and in vitro transcribed Alu RNA. The coding sequence of Δhel-DICER1 is 3.9 kb, which is compatible with efficient AAV packaging.

A Δhel-DICER1 coding sequence was cloned into the pAAV-MCS packaging vector, with a total packaging genome size including regulatory elements and AAV inverted terminal repeats (ITR) of 5.0 kb. Transient transfection of Δhel-DICER1 resulted in robust expression in HeLa cells as detected by immunoblotting. However, transfection into human RPE cells resulted in no detectable expression of the Δhel-DICER1 protein. Moreover, endogenous DICER1 abundance in hRPE cells was diminished by Δhel-DICER1 plasmid transfection. Therefore, it appeared that DICER1 expression in RPE was subject to negative auto-regulation, potentially arising due to the enhanced miRNA processing activity of DICER1. Consistent with this hypothesis, transient Δhel-DICER1 expression was observed within 4 hours of transfection, but reduced to undetectable levels soon thereafter. Further, co-transfection with double-stranded RNA, which can compete with DICER1 processing and RISC loading, restored Δhel-DICER1 expression to detectable levels, suggesting that impaired Δhel-DICER1 expression could have been due to negative feedback via RNA interference.

Because the Δhel-DICER1 sequence employed lacked the DICER1 3′-UTR, it was possible that miRNA binding sites within the coding sequence were responsible for the downregulation of expression observed. Certain let-7 miRNAs were known to specifically target the DICER1 coding region, with three putative target regions being identified. To reduce or eliminate this regulatory mechanism, various let-7-resistant Δhel-DICER1 coding sequences with silent mutations of these three targets were generated. Although let-7-resistant Δhel-DICER1 was robustly expressed in Hela cells, it too failed to express in human RPE cells in detectable levels. Therefore, a pan-miRNA-resistant Δhel-DICER1 was constructed in which 33 miRNA seed sequences (28 human and 5 mouse) within the Δhel-DICER1 coding region were modified with silent mutations, and codon optimization was employed to make additional silent mutations in the wild type human DICER1 coding sequence. The resulting construct is referred to herein as OptiDicer, and it exhibited robust and stable expression in hRPE cells.

Gene delivery of OptiDicer improves spontaneous chorioretinal neovascularization (CRNV) in mice. To determine whether DICER1 gene delivery affected CRNV in JR5558 mice, an adeno-associated vector (AAV) gene transfer strategy, a modality that has demonstrated safety and efficacy in treating blinding diseases in human patients and in experimental models of choroidal and retinal neovascularization, was employed. Stable expression of AAV-encoded OptiDicer was confirmed in retina of JR5558 mice. Fluorescein angiograms (FA) were collected from naïve 6-week old JR5558 mice with established CNV. Then, AAV-OptiDicer or an empty AAV2-control was administered by subretinal injection in contralateral eyes. Fourteen and twenty-eight days after injections, follow-up FA revealed significant improvement in both the frequency and severity of neovascular lesions within injected areas compared to eyes transduced with a control vector, suggesting that subretinal delivery of a bioactive DICER1 variant by AAV antagonized CRNV in JR5558 mice.

Thus, disclosed herein is that genetic suppression of Dicer1 in two independent mouse models manifested in the eye as focal RPE atrophy and aberrant choroidal and retinal neovascularization, and that DICER1 expression was reduced in a mouse model of spontaneous CNV. Further disclosed is that AAV-enforced expression of a novel DICER1 construct, which successfully escaped miRNA negative feedback, reduced spontaneous CNV in mice. In addition to expanding upon prior studies of DICER1 loss in atrophic AMD, these findings identified maintenance of outer retinal avascularity as another critical function of DICER1 in maintaining retinal homeostasis.

The established role of DICER1 in mediating developmental and pathological angiogenesis and neovascularization is largely context- and tissue type-dependent. For example, whereas DICER1 ablation prevents developmental and postnatal angiogenesis in multiple diverse settings (Yang et al., 2005; Kuehbacher et al., 2007; Suarez et al., 2008; Plummer et al., 2013; Chen et al., 2014), DICER1 deficiency can promote neovascularization in stroke (Li et al., 2015), angiosarcoma (Hanna et al., 2017), and renal cell carcinoma (Chen et al., 2016; Fan et al., 2016). Further, exogenous delivery of DICER1 suppresses tumor angiogenesis (Fan et al., 2016) and hypoxia-induced angiogenic responses in human endothelial cells (Grunin et al., 2012). The present findings suggested that in the outer retina, DICER1 expression served to prevent pathological neovascularization.

The downstream effects of DICER1 downregulation, including modulation of angiogenesis, have most commonly been attributed to loss of miRNA biogenesis. It will be important in future work to establish whether miRNA or non-canonical DICER1 substrates such as Alu RNAs, which promote RPE degeneration due to DICER1 loss, also contribute to neovascular and degenerative phenotypes observed in this study.

Because of the unique features of the Dicer1^{d/d} mouse line, including exhibiting multiple AMD-related pathologies such as DICER1 deficiency, inflammasome activation and dependence, relatively early onset of phenotypes, age-dependence of pathological incidence and severity, and facile phenotypic scoring, this model may be of interest to both basic discovery and translational research as a preclinical testing platform. This study also suggests that restoring DICER1 expression in the retina could itself be a viable therapeutic target in physiologic and pathologic conditions.

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Alsaleh et al. (2016) Reduced DICER1 Expression Bestows Rheumatoid Arthritis Synoviocytes Proinflammatory Properties and Resistance to Apoptotic Stimuli. Arthritis Rheumatol 68(8):1839-1848.
Altschul et al. (1990a) Basic local alignment search tool. J Mol Biol 215:403-410.
Altschul et al. (1990b) Protein database searches for multiple alignments. Proc Natl Acad Sci USA 87:14:5509-13.
Altschul et al. (1997) Gapped BLAST and PSI-BLAST: a new generation of protein database search programs. Nucleic Acids Res 25:3389-3402.
Ausubel et al. (1992) Current Protocols in Molecular Biology, John Wylie & Sons, Inc., New York, N.Y., United States of America.
Babiarz et al. (2008) Mouse ES cells express endogenous shRNAs, siRNAs, and other Microprocessor-independent, Dicer-dependent small RNAs. Genes Dev. 22(20):2773-2785.
Bainbridge et al. (2008) Effect of gene therapy on visual function in Leber's congenital amaurosis. N Engl J Med 358(21):2231-2239.
Bernstein et al. (2001) Role for a bidentate ribonuclease in the initiation step of RNA interference. Nature 409(6818): 363-366.
Bernstein et al. (2003) Dicer is essential for mouse development. Nat. Genet. 35(3):215-217.
Chang et al. (2018) Spontaneous Posterior Segment Vascular Disease Phenotype of a Mouse Model, rnv3, Is Dependent on the Crb1rd8 Allele. Invest Ophthalmol Vis Sci 59(12):5127-5139.
Chen et al. (2014) Global microRNA depletion suppresses tumor angiogenesis. Genes Dev. 28(10):1054-1067.
Chen et al. (2016) Dicer suppresses MMP-2-mediated invasion and VEGFA-induced angiogenesis and serves as a promising prognostic biomarker in human clear cell renal cell carcinoma. Oncotarget 7(51):84299-84313.
Clark et al. (1995) Cell lines for the production of recombinant adeno-associated virus. Hum Gene Ther 6:1329-1341.
Curcio (2018) Soft Drusen in Age-Related Macular Degeneration: Biology and Targeting Via the Oil Spill Strategies. Invest Ophthalmol Vis Sci 59(4):AMD160-AMD181.
Devereux et al. (1984) A comprehensive set of sequence analysis programs for the VAX. Nucleic Acids Research 12:387-395.
Dong et al. (1996) Quantitative analysis of the packaging capacity of recombinant adeno-associated virus. Hum. Gene Ther 7(17):2101-2112.
Dridi et al. (2012) ERK1/2 activation is a therapeutic target in age-related macular degeneration. Proc Natl Acad Sci USA 109(34):13781-13786.
Du et al. (2008) Structural and biochemical insights into the dicing mechanism of mouse Dicer: a conserved lysine is critical for dsRNA cleavage. Proc Natl Acad Sci USA 105(7):2391-2396.
Fan et al. (2016) Dicer suppresses the malignant phenotype in VHL-deficient clear cell renal cell carcinoma by inhibiting HIF-2alpha. Oncotarget 7(14):18280-18294.
Fareh et al. (2016) TRBP ensures efficient Dicer processing of precursor microRNA in RNA-crowded environments. Nat Commun 7:13694.
Flemr et al. (2013) A retrotransposon-driven dicer isoform directs endogenous small interfering RNA production in mouse oocytes. Cell 155(4):807-816.
Forman et al. (2008) A search for conserved sequences in coding regions reveals that the let-7 microRNA targets Dicer within its coding sequence. Proc Natl Acad Sci USA 105(39):14879-14884.
Fowler et al. (2014) Nucleoside reverse transcriptase inhibitors possess intrinsic anti-inflammatory activity. Science 346(6212):1000-1003.
Foxton et al. (2016) Distal retinal ganglion cell axon transport loss and activation of p38 MAPK stress pathway following VEGF-A antagonism. Cell Death Dis 7:e2212.
Fukasawa et al. (2006) Genomic imprinting in Dicer1-hypomorphic mice. Cytogenet. Genome Res. 113 (1-4): 138-143.
Gan et al. (2006) Structural insight into the mechanism of double-stranded RNA processing by ribonuclease III. Cell 124(2):355-366.
Gao et al. (1998) High-titer adeno-associated viral vectors from a Rep/Cap cell line and hybrid shuttle virus. Gene Ther 9:2353-2362.
Gelfand et al. (2015) Iron Toxicity in the Retina Requires Alu RNA and the NLRP3 Inflammasome. Cell Rep 11(11):1686-1693.
GENBANK® Accession Nos. NC_000014.9; NC_000078.6; NM_001195573.1; NM_001271282.3; NM_001291628.1; NM_030621.4; NM_133239.2; NM_148948.2; NM_148948.2; NM_177438.2; NM_177438.2; NP_001182502.1; NP_001258211.1; NP_001278557.1; NP_085124.2; NP_683750.2; NP_683750.2; NP_803187.1; NP_803187.1.
Grimm et al. (1998) Novel tools for production and purification of recombinant adenoassociated virus vectors. Hum Gene Ther 9:2745-2760.
Grunin et al. (2012) Chemokine receptor expression in peripheral blood monocytes from patients with neovascular age-related macular degeneration. Invest Ophthalmol Vis Sci 53(9):5292-5300.
Gurtan et al. (2012) In vivo structure-function analysis of human Dicer reveals directional processing of precursor miRNAs. RNA 18(6):1116-1122.
Hanna et al. (2017) Biallelic Dicer1 Loss Mediated by aP2-Cre Drives Angiosarcoma. Cancer Res. 77(22):6109-6118.
Hasegawa et al. (2014) Characterization of a Spontaneous Retinal Neovascular Mouse Model. PLoS One 9(9):e106507.
Henikoff & Henikoff (1992) Amino acid substitution matrices from protein blocks. Proc Natl Acad Sci USA 89:10915-10919.
Hoerster et al. (2012) In-vivo and ex-vivo characterization of laser-induced choroidal neovascularization variability in mice. Graefes Arch Clin Exp Ophthalmol 250(11): 1579-1586.
Hu et al. (2012) DICER- and AGO3-dependent generation of retinoic acid-induced DR2 Alu RNAs regulates human stem cell proliferation. Nat. Struct. Mol. Biol. 19(11):1168-1175.
Inoue & Russell (1998) Packaging cells based on inducible gene amplification for the production of adeno-associated virus vectors. J Virol 72:7024-7031.
Kaneko et al. (2011) DICER1 deficit induces Alu RNA toxicity in age-related macular degeneration. Nature 471(7338):325-330.
Karlin & Altschul (1993) Applications and statistics for multiple high-scoring segments in molecular sequences. Proc Natl Acad Sci USA 90:5873-5877.
Kaszubski et al. (2016) Geographic Atrophy and Choroidal Neovascularization in the Same Eye: A Review. Ophthalmic Res. 55(4):185-193.
Kennedy et al. (2015) Production of functional small interfering RNAs by an amino-terminal deletion mutant of human Dicer. Proc Natl Acad Sci USA 112(50):E6945-6954.
Kerur et al. (2013) TLR-independent and P2X7-dependent signaling mediate Alu RNA-induced NLRP3 inflammasome activation in geographic atrophy. Invest Ophthalmol Vis Sci 54(12):7395-7401.
Kerur et al. (2018) cGAS drives noncanonical-inflammasome activation in age-related macular degeneration. Nat. Med. 24(1):50-61.
Kim et al. (2014) DICER1/Alu RNA dysmetabolism induces Caspase-8-mediated cell death in age-related macular degeneration. Proc Natl Acad Sci USA 111(45):16082-16087.
Kim et al. (2016) Re-evaluation of the roles of DROSHA, Export in 5, and DICER in microRNA biogenesis. Proc Natl Acad Sci USA 113(13):E1881-1889.
Kuehbacher et al. (2007) Role of Dicer and Drosha for endothelial microRNA expression and angiogenesis. Circ Res 101(1):59-68.
Kuida et al. (1995) Altered cytokine export and apoptosis in mice deficient in interleukin-1 beta converting enzyme. Science 267(5206):2000-2003.
Lai et al. (2005) Long-term evaluation of AAV-mediated sFlt-1 gene therapy for ocular neovascularization in mice and monkeys. Mol. Ther. 12(4):659-668.
Lee et al. (2018) Transduction Patterns of Adeno-associated Viral Vectors in a Laser-Induced Choroidal Neovascularization Mouse Model. Mol Ther Methods Clin Dev 9:90-98.
Li et al. (2015) MicroRNA-107 contributes to post-stroke angiogenesis by targeting Dicer-1. Sci Rep 5:13316.
Liang et al. (2013) Transfection of siRNAs can alter miRNA levels and trigger non-specific protein degradation in mammalian cells. Biochim. Biophys. Acta 1829(5):455-468.
Liu & Wang (2019) Prediction of functional microRNA targets by integrative modeling of microRNA binding and target expression data. Genome Biology. 20(1):18.
Liu et al. (1999) Production of recombinant adeno-associated virus vectors using a packaging cell line and a hybrid recombinant adenovirus. Gene Ther 6:293-299.
Luo et al. (2013) Photoreceptor avascular privilege is shielded by soluble VEGF receptor-1. Elife 2:e00324.
Ma et al. (2004) Structural basis for overhang-specific small interfering RNA recognition by the PAZ domain. Nature 429(6989):318-322.
Ma et al. (2008) Autoinhibition of human dicer by its internal helicase domain. J. Mol. Biol. 380(1):237-243.
MacRae et al. (2007) Structural determinants of RNA recognition and cleavage by Dicer. Nat. Struct. Mol. Biol. 14(10):934-940.
Maguire et al. (2008) Safety and efficacy of gene transfer for Leber's congenital amaurosis. N Engl J Med 358(21):2240-2248.
Matsushita et al. (1998) Adeno-associated virus vectors can be efficiently produced without helper virus. Gene Ther 5:938-945.
Mattapallil et al. (2012) The Rd8 mutation of the Crb1 gene is present in vendor lines of C57BL/6N mice and embryonic stem cells, and confounds ocular induced mutant phenotypes. Invest Ophthalmol Vis Sci 53(6):2921-2927.
Morita et al. (2009) Dicer is required for maintaining adult pancreas. PLoS One 4(1):e4212.
Murchison et al. (2007) Critical roles for Dicer in the female germline. Genes Dev. 21(6):682-693.
Muzyczka (1992) Use of adeno-associated virus as a general transduction vector for mammalian cells. Curr Topics Microbiol Immunol 158:97-129.
Nagai et al. (2014) Spontaneous CNV in a novel mutant mouse is associated with early VEGF-A-driven angiogenesis and late-stage focal edema, neural cell loss, and dysfunction. Invest Ophthalmol Vis Sci 55(6):3709-3719.
Nagai et al. (2015) Novel CCR3 Antagonists Are Effective Mono- and Combination Inhibitors of Choroidal Neovascular Growth and Vascular Permeability. Am J Pathol 185(9):2534-2549.
Needleman & Wunsch (1970) A general method applicable to the search for similarities in the amino acid sequence of two proteins. J Mol Biol 48:443-453.
Ohnishi et al. (2012) Active role of small non-coding RNAs derived from SINE/B1 retrotransposon during early mouse development. Mol. Biol. Rep. 39(2):903-909.
Ostermann et al. (2012) Deregulation of type I IFN-dependent genes correlates with increased susceptibility to cytomegalovirus acute infection of dicer mutant mice. PLoS One 7(8):e43744.
Ostermann et al. (2015) Increased Viral Dissemination in the Brain and Lethality in MCMV-Infected, Dicer-Deficient Neonates. Viruses 7(5):2308-2320.
Otsuka et al. (2007) Hypersusceptibility to vesicular stomatitis virus infection in Dicer1-deficient mice is due to impaired miR24 and miR93 expression. Immunity 27(1):123-134.
Otsuka et al. (2008) Impaired microRNA processing causes corpus luteum insufficiency and infertility in mice. J. Clin. Invest. 118(5):1944-1954.
Paneghetti & Ng (2016) A novel endothelial-derived anti-inflammatory activity significantly inhibits spontaneous choroidal neovascularisation in a mouse model. Vasc Cell 8:2.
Park et al. (2011) Dicer recognizes the 5′ end of RNA for efficient and accurate processing. Nature 475(7355):201-205.
Plummer et al. (2013) MicroRNAs regulate tumor angiogenesis modulated by endothelial progenitor cells. Cancer Res. 73 (1): 341-352.
Ren et al. (2012) Dicer-dependent biogenesis of small RNAs derived from 7SL RNA. PLoS One 7(7):e40705.
Robertson et al. (2018) DICER1 Syndrome: DICER1 Mutations in Rare Cancers. Cancers (Basel) 10(5): 143.
Schnabolk et al. (2018) Delivery of CR2-fH Using AAV Vector Therapy as Treatment Strategy in the Mouse Model of Choroidal Neovascularization. Mol Ther Methods Clin Dev 9:1-11.
Smith & Waterman (1981) Comparison of biosequences. Adv Appl Math 2:482-489.
Suarez et al. (2008) Dicer-dependent endothelial microRNAs are necessary for postnatal angiogenesis. Proc Natl Acad Sci USA 105(37):14082-14087.
Sun et al. (2017a) Inflammatory signals from photoreceptor modulate pathological retinal angiogenesis via c-Fos. J. Exp. Med. 214(6):1753-1767.
Sun et al. (2017b) Sema3f Protects Against Subretinal Neovascularization In Vivo. EBioMedicine 18:281-287.
Sundermeier et al. (2017) MicroRNA-processing Enzymes Are Essential for Survival and Function of Mature Retinal Pigmented Epithelial Cells in Mice. J. Biol. Chem. 292(8):3366-3378.
Sunness et al. (1999) The development of choroidal neovascularization in eyes with the geographic atrophy form of age-related macular degeneration. Ophthalmology 106(5):910-919.
Tamayose et al. (1996) A new strategy for large-scale preparation of high-titer recombinant adeno-associated virus vectors by using packaging cell lines and sulfonated cellulose column chromatography. Hum Gene Ther 7:507-513.
Tarallo et al. (2012) DICER1 loss and Alu RNA induce age-related macular degeneration via the NLRP3 inflammasome and MyD88. Cell 149(4):847-859.
U.S. Patent Application Publication Nos. 2008/0206812; 2017/0157267; 2018/0311290; 2019/0002916; 2019/0048362; 2019/0060489.
U.S. Pat. Nos. 7,745,389; 8,664,176; 8,809,058; 9,314,453; 9,540,659; 9,701,984; 9,840,719; 10,004,788; 10,117,931; 10,214,572; 10,392,632.
Wang (2008) miRDB: a microRNA target prediction and functional annotation database with a wiki interface. RNA 14(6):1012-1017.
Wei et al. (2016) An easy, rapid method to isolate RPE cell protein from the mouse eye. Exp Eye Res 145:450-455.
Wong & Wang (2015) miRDB: an online resource for microRNA target prediction and functional annotations. Nucleic Acids Res. 43:D146-152.
Wong et al. (2014) Global prevalence of age-related macular degeneration and disease burden projection for 2020 and 2040: a systematic review and meta-analysis. Lancet Glob Health 2(2):e106-116.
Wu et al. (2010) Effect of genome size on AAV vector packaging. Mol. Ther. 18(1):80-86.
Xiao et al. (1998) Adeno-associated virus as a vector for liver-directed gene therapy. J Virol 72:10222-10226.
Yang et al. (1994) Characterization of cell lines that inducibly express the adeno-associated virus Rep proteins. J Virol 68:4847-4856.
Yang et al. (2005) Dicer is required for embryonic angiogenesis during mouse development. J. Biol. Chem. 280(10):9330-9335.
Yu et al. (2008) Increased choroidal neovascularization following laser induction in mice lacking lysyl oxidase-like 1. Invest Ophthalmol Vis Sci 49(6):2599-2605.

It will be understood that various details of the presently disclosed subject matter can be changed without departing from the scope of the presently disclosed subject matter. Furthermore, the foregoing description is for the purpose of illustration only, and not for the purpose of limitation.

1. A nucleotide sequence encoding a polypeptide with ribonuclease III activity, the nucleotide sequence being at least 50%, 55%, 60%, 65%, 70%, 75%, 80%, 85%, 90%, 95%, 96%, 97%, 98%, or 99% percent identical to SEQ ID NO: 20 or SEQ ID NO: 22, wherein the polypeptide is at least 90%, 95%, 96%, 97%, 98%, or 99% percent identical to SEQ ID NO: 23.

2. The nucleotide sequence of claim 1, wherein as compared to SEQ ID NO: 20, the nucleotide sequence comprises one or more nucleotide substitutions in one or more of the nucleotide positions of SEQ ID NO: 20 identified in Table 2, and further wherein the one or more nucleotide substitutions reduce or eliminate regulation of expression of an mRNA transcribed from SEQ ID NO: 20 by a member of an miRNA family listed in Table 2.

3. The nucleotide sequence of claim 1, wherein as compared to SEQ ID NO: 20, the nucleotide sequence comprises one or more nucleotide substitutions within one or more of nucleotide positions 571-578, 778-784, 1784-1791, 1892-1899, and/or 3282-3289 of SEQ ID NO: 20, wherein the one or more nucleotide substitutions reduce or eliminate regulation of expression of an mRNA transcribed from SEQ ID NO: 20 by a member of the let-7 family of miRNAs.

4. The nucleotide sequence of claim 1, wherein the one or more nucleotide substitutions is/are silent with respect to the amino acid encoded by a codon comprising the one or more nucleotide substitutions as compared to the corresponding codon in SEQ ID NO: 20.

5. The nucleotide sequence of claim 1, wherein the nucleotide sequence comprises one or more nucleotide substitutions within one or more of the nucleotide positions of SEQ ID NO: 20 identified in Table 2, optionally within one or more of nucleotide positions 571-578, 778-784, 1784-1791, 1892-1899, and 3282-3289 of SEQ ID NO: 20, and further wherein the one or more nucleotide substitutions reduce or eliminate regulation of expression of an mRNA transcribed from SEQ ID NO: 20 by a member of an miRNA family listed in Table 2, optionally a member of the let-7 family of miRNAs, and further wherein the one or more nucleotide substitutions is/are silent with respect to the amino acid encoded by a codon comprising the one or more nucleotide substitutions as compared to the corresponding codon in SEQ ID NO: 20.

6. The nucleotide sequence of claim 5, wherein the nucleotide sequence comprises one or more nucleotide substitutions within each of nucleotide position ranges 571-578, 778-784, 1784-1791, 1892-1899, and 3282-3289 of SEQ ID NO: 20.

7. The nucleotide sequence of claim 6, wherein the nucleotide sequence encodes SEQ ID No: 23.

8. The nucleotide sequence of claim 6, wherein as compared to SEQ ID NO: 20, the nucleotide sequence comprises one or more nucleotide substitutions designed for codon optimization of the nucleotide sequence, optionally wherein the codon optimization is with respect to an expression of the nucleotide sequence in a human cell.

9. The nucleotide sequence of claim 1, wherein the nucleotide sequence encodes a polypeptide comprising, consisting essentially of, or consisting of an amino acid sequence at least 95% identical to SEQ ID NO: 23, wherein as compared to SEQ ID NO: 23, the nucleotide sequence encodes one or more conservative amino acid substitutions only.

10. The nucleotide sequence of claim 1, wherein the nucleotide sequence encodes a polypeptide comprising, consisting essentially of, or consisting of the amino acid sequence set forth in SEQ ID NO: 23.

11. A vector, optionally an expression vector, comprising or consisting essentially of the nucleotide sequence of claim 1.

12. The vector, optionally the expression vector, of claim 11, wherein the vector is an AAV vector.

13. A host cell comprising the vector of claim 11.

14. A pharmaceutical composition comprising the vector of claim 11 and a pharmaceutically acceptable diluent and/or excipient, optionally wherein the pharmaceutically acceptable diluent and/or excipient is pharmaceutically acceptable for use in a human.

15. A method for expressing a Δhel-DICER1 polypeptide in a cell, optionally a cell of the eye, further optionally an RPE cell, the method comprising introducing into the cell the pharmaceutical composition of claim 14.

16. A method for preventing and/or treating development of a disease or disorder associated with undesirably low DICER1 expression, the method comprising introducing into the eye, retina, and/or RPE the pharmaceutical composition of claim 14.

17. The method of claim 16, wherein the undesirably low DICER1 expression occurs in the eye, optionally the retina, further optionally the RPE.

18. The method of claim 16, wherein the disease or disorder associated with undesirably low DICER1 expression is selected from the group consisting of DICER1 syndrome, type 2 diabetes mellitus, diabetic retinopathy, age-related macular degeneration (AMD), RPE degeneration, aberrant choroidal and retinal neovascularization (CRNV), subretinal and retinal fibrosis, Fuchs' endothelial corneal dystrophy, Alzheimer's disease, rheumatoid arthritis, lupus, renal injury, tubulointerstitial fibrosis, glial axonal degeneration, idiopathic pulmonary fibrosis, lipid dysregulation, cholesterol accumulation associated with non-alcoholic steatohepatitis, clear cell renal cell carcinoma, atopic dermatitis, glomerulopathy, disorders of hypomyelination, tubal ectopic pregnancy and tubal abnormalities such as but not limited to cysts and disorganization of epithelial cells and smooth muscle cells, amyotrophic lateral sclerosis (ALS), Duchenne's muscular dystrophy, Sertoli cell deficiency/impaired spermatogenesis, and combinations thereof.

19. The method of claim 18, wherein the disease or disorder is age-related macular degeneration (AMD).

20. The method of claim 18, wherein the disease or disorder of the eye is associated with RPE degeneration, aberrant choroidal and retinal neovascularization (CRNV), or both.

21. A method for restoring undesirably low DICER1 expression, optionally in the eye, further optionally the retina, of a subject in need thereof, the method comprising administering to the subject the pharmaceutical composition of claim 14.

22. The method of claim 15, wherein the pharmaceutical composition is administered by intravitreous injection; subretinal injection; episcleral injection; sub-Tenon's injection; retrobulbar injection; peribulbar injection; topical eye drop application; release from a sustained release implant device that is sutured to or attached to or placed on the sclera, or injected into the vitreous humor, or injected into the anterior chamber, or implanted in the lens bag or capsule; oral administration, intravenous administration; intramuscular injection; intraparenchymal injection; intracranial administration; intraarticular injection; retrograde ureteral infusion; intrauterine injection; intratesticular tubule injection; or any combination thereof.

23. Use of the pharmaceutical composition of claim 14 to express a Δhel-DICER1 polypeptide in a cell, optionally a cell of the eye, further optionally an RPE cell.

24. The use of claim 23, wherein the cell is a human cell.

25. Use of the pharmaceutical composition of claim 14 to prevent and/or treat development of a disease or disorder, optionally of the eye, further optionally the retina, further optionally the RPE, wherein the disease or disorder of the eye is associated with undesirably low DICER1 expression.

26. The use of claim 25, wherein the disease or disorder associated with undesirably low DICER1 expression is selected from the group consisting of DICER1 syndrome, type 2 diabetes mellitus, diabetic retinopathy, age-related macular degeneration (AMD), RPE degeneration, aberrant choroidal and retinal neovascularization (CRNV), subretinal and retinal fibrosis, Fuchs' endothelial corneal dystrophy, Alzheimer's disease, rheumatoid arthritis, lupus, renal injury, tubulointerstitial fibrosis, glial axonal degeneration, idiopathic pulmonary fibrosis, lipid dysregulation, cholesterol accumulation associated with non-alcoholic steatohepatitis, clear cell renal cell carcinoma, atopic dermatitis, glomerulopathy, disorders of hypomyelination, tubal ectopic pregnancy and tubal abnormalities such as but not limited to cysts and disorganization of epithelial cells and smooth muscle cells, amyotrophic lateral sclerosis (ALS), Duchenne's muscular dystrophy, Sertoli cell deficiency/impaired spermatogenesis, and combinations thereof.

27. The use of claim 26, wherein the disease or disorder is age-related macular degeneration (AMD).

28. The use of claim 26, wherein the disease or disorder of the eye is associated with RPE degeneration, aberrant choroidal and retinal neovascularization (CRNV), or both.

29. Use of the vector of claim 12 to restore undesirably low DICER1 expression in a cell in need thereof, optionally a cell of the eye, further optionally a cell of the retina, of a subject.

30. A pharmaceutical composition for preventing and/or treating a disease or disorder associated with undesirably low DICER1 expression, optionally undesirably low DICER1 expression in the eye, further optionally in the retina, further optionally in the RPE, of a subject in need thereof, the pharmaceutical composition comprising an effective amount of the pharmaceutical composition of claim 14.

31. The pharmaceutical composition of claim 30, wherein the disease or disorder associated with undesirably low DICER1 expression is selected from the group consisting of DICER1 syndrome, type 2 diabetes mellitus, diabetic retinopathy, age-related macular degeneration (AMD), RPE degeneration, aberrant choroidal and retinal neovascularization (CRNV), subretinal and retinal fibrosis, Fuchs' endothelial corneal dystrophy, Alzheimer's disease, rheumatoid arthritis, lupus, renal injury, tubulointerstitial fibrosis, glial axonal degeneration, idiopathic pulmonary fibrosis, lipid dysregulation, cholesterol accumulation associated with non-alcoholic steatohepatitis, clear cell renal cell carcinoma, atopic dermatitis, glomerulopathy, disorders of hypomyelination, tubal ectopic pregnancy and tubal abnormalities such as but not limited to cysts and disorganization of epithelial cells and smooth muscle cells, amyotrophic lateral sclerosis (ALS), Duchenne's muscular dystrophy, Sertoli cell deficiency/impaired spermatogenesis, and combinations thereof.

32. The pharmaceutical composition of claim 31, wherein the disease or disorder is age-related macular degeneration (AMD).

33. The pharmaceutical composition of claim 31, wherein the disease or disorder is associated with RPE degeneration, aberrant choroidal and/or retinal neovascularization (CRNV), or both.

34. The pharmaceutical composition of claim 31, wherein the effective amount restores undesirably low DICER1 expression in a cell, tissue, or organ in which it occurs, optionally a cell or tissue of the eye, further optionally the retina, of the subject.

35. The pharmaceutical composition of claim 30, wherein the pharmaceutical composition is formulated for administration by intravitreous injection; subretinal injection; episcleral injection; sub-Tenon's injection; retrobulbar injection; peribulbar injection; topical eye drop application; release from a sustained release implant device that is sutured to or attached to or placed on the sclera, or injected into the vitreous humor, or injected into the anterior chamber, or implanted in the lens bag or capsule; oral administration, intravenous administration; intramuscular injection; intraparenchymal injection; intracranial administration; intraarticular injection; retrograde ureteral infusion; intrauterine injection; intratesticular tubule injection;
and any combination thereof.
